SEQUENCE LISTING (5 sequences)

SEQ ID NO: 1 - protein, 11 aa, Artificial Sequence
  Synthetic peptide
DXGNTAGVYTK

SEQ ID NO: 2 - protein, 18 aa, Artificial Sequence
  Synthetic peptide
DXIIAEGANGATTAEAEK

SEQ ID NO: 3 - protein, 23 aa, Artificial Sequence
  Synthetic peptide
DXGLGEHNIDVLEGNEFDINAAK

SEQ ID NO: 4 - protein, 21 aa, Artificial Sequence
  Synthetic peptide
GLGEHNIDVLEGNEQFINAAK

SEQ ID NO: 5 - protein, 7 aa, Artificial Sequence
  Synthetic peptide
AFLDASK

(19) United States
(12) Patent Application Publication
(10) Pub. No.: US 2011/0143951 A1
(43) Pub. Date: Jun. 16, 2011

(54) MASS MARKERS AND METHODS

(75) Inventor: Andrew H. Thompson, Cambridge (GB)

(73) Assignee: Brax Limited, Hertford (GB)

(21) Appl. No.: 12/999,934

(22) PCT Filed: Jun. 19, 2009
(86) PCT No.: PCT/GB09/01558

§ 371 Date: Mar. 8, 2011

(30) Foreign Application Priority Data
Jun. 19, 2008 (GB) 0811298.9

(51) Int. Cl. C40B 30/00; G01N 33/53; G01N 21/00; C40B 40/04; C40B 40/10; C07K 2/00; C07D 241/04; C07D 207/14; C07D 207/46; C07C 229/00

(52) U.S. Cl. 506/7; 435/7.1; 436/164; 506/15; 506/18; 530/300; 544/358; 548/532; 548/542; 560/155; 562/553; 562/574

(57) ABSTRACT

The invention describes compounds useful for labelling molecules of interest (i.e. analytes), particularly biomolecules such as peptides, proteins, oligonucleotides and nucleic acids, and also methods for analysing, detecting and/or isolating these labelled molecules using mass spectrometry. The compound in one aspect is a mass marker for labelling of an analyte detectable by mass spectrometry such as neutral loss mass spectroscopy, in which the mass marker comprises a neutral loss mass modifier linked via a first collision cleavable linker to a reactive group having reactive functionality for attachment to the analyte. The neutral loss mass modifier upon cleavage from the analyte during mass spectroscopy is uncharged.

This invention relates to compounds useful for labelling molecules of interest, i.e. analytes, particularly biomolecules such as peptides, proteins, oligonucleotides and nucleic acids, and also methods for analysing, detecting and/or isolating such molecules using mass spectrometry.

Various methods of labelling molecules of interest are known in the art, including radioactive atoms, fluorescent dyes, luminescent reagents, electron capture reagents and light absorbing dyes. Each of these labelling methods has features which make it suitable for certain applications and not others. For reasons of safety, interest in non-radioactive labelling methods lead to the widespread commercial development of fluorescent labelling schemes particularly for genetic analysis. Fluorescent labelling methods permit the labelling of a relatively small number of molecules simultaneously, typically four labels can be used simultaneously and possibly up to eight. However the costs of the detection apparatus and the difficulties of analysing the resultant signals limit the number of labels that can be used simultaneously in a fluorescence detection scheme.

More recently there has been development in the area of mass spectrometry as a method of detecting labels that are cleavably attached to their associated molecule of interest. In many molecular biology applications, separation of molecules of interest is required prior to analysis. These are generally liquid phase separations. Mass spectrometry has developed a number of interfaces for liquid phase separations which make mass spectrometry particularly effective as a detection system for these kinds of applications. Liquid Chromatography Mass Spectrometry (LC-MS) has been used to detect analyte ions or their fragment ions directly. However, for many applications such as nucleic acid analysis, the structure of the analyte can be determined from indirect labelling. This is advantageous particularly with respect to the use of mass spectrometry because complex biomolecules such as DNA have complex mass spectra and are detected directly with relatively poor sensitivity. Indirect detection means that an associated label molecule can be used to identify the original analyte, the label being designed for sensitive detection and a simple mass spectrum. Simple mass spectra allow multiple labels to be used to analyse multiple analytes simultaneously.

WO98/31830 describes arrays of nucleic acid probes covalently attached to cleavable labels that are detectable by mass spectrometry which identify the sequence of the covalently linked nucleic acid probe. The labelled probes of this application have the structure Nu-L-M where Nu is a nucleic acid covalently linked to L, a cleavable linker, covalently linked to M, a mass label. Preferred cleavable linkers in this application cleave within the ion source of the mass spectrometer. Preferred mass labels are substituted poly-aryl ethers. WO98/31830 discloses a variety of ionisation methods and analysis by quadrupole mass analysers, Time of Flight (TOF) analysers and magnetic sector instruments as specific methods of analysing mass labels by mass spectrometry.

WO95/04160 discloses ligands, and specifically nucleic acids, cleavably linked to mass tag molecules. Preferred cleavable linkers are photo-cleavable. This application discloses Matrix Assisted Laser Desorption Ionisation (MALDI) Time of Flight (TOF) mass spectrometry as a specific method of analysing mass labels by mass spectrometry.

WO98/26095 discloses releasable non-volatile mass-label molecules. In preferred embodiments these labels comprise polymers, typically biopolymers, which are cleavably attached to a reactive group or ligand, i.e. a probe. Preferred cleavable linkers appear to be chemically or enzymatically cleavable. This application discloses MALDI TOF mass spectrometry as a specific method of analysing mass labels by mass spectrometry.

WO97/27327, WO97/27325 and WO97/27331 disclose ligands, particularly nucleic acids, cleavably linked to mass tag molecules. Preferred cleavable linkers appear to be chemically or photo-cleavable. These prior art documents disclose a variety of ionisation methods and analysis by quadrupole mass analysers, TOF analysers and magnetic sector instruments as specific methods of analysing mass labels by mass spectrometry.

WO01/68664 and WO03/025576 disclose organic molecule mass markers that are analysed by tandem mass spectrometry. The mass markers have a mass tag component and a mass normalisation component that are connected to each other by a collision cleavable group. Sets of tags can be synthesised where the sum of the masses of the two components produce markers with the same overall mass. The mass markers are typically analysed after cleavage from their analyte. Analysis takes place in an instrument capable of tandem mass spectrometric analysis. In the first stage of analysis, the mass of the mass marker comprising both the mass tag and mass normaliser are selected by the first mass analyser of the tandem instrument, which allows the markers to be abstracted from the background. Collision of the markers in the second stage of the instrument separates the two components of the tag from each other. Only the mass tag components are detected in the third mass analyser. This allows confirmation that the peak selected in the first analyser is a mass marked peptide. The whole process is stated to greatly enhance the signal to noise ratio of the analysis and improves sensitivity. This mass marker design also compresses the mass range over which an array of mass markers is spread as mass markers can have the same mass as long as they give rise to mass tag fragments that are uniquely resolvable. Moreover, with isotopes this mass marker design allows the synthesis of markers which are chemically identical and have the same mass but which are still resolvable by mass spectrometry. Use of these markers to identify oligonucleotide probes is described.

Thus, the prior art provides analytes cleavably linked to tags where the tags are cleaved and then the tags are detected by mass spectrometry. The use of these mass tags enables multiplexing of biological assays.

Gygi et al. (1999, Nature Biotechnology 17: 994-999, and see also WO00/11208) disclose the use of “isotope encoded affinity tags” (ICAT's) for the capture of peptides from proteins, to allow protein expression analysis. In this article, the authors describe the use of a biotin linker which is reactive to thiols, for the capture of peptides comprising cysteine. A sample of protein from one source is reacted with the biotin linker and cleaved with an endopeptidase. The biotinylated cysteine-containing peptides can then be isolated on avidinated beads for subsequent analysis by mass spectrometry. Two samples can be compared quantitatively by labelling one sample with the biotin linker and labelling the second sample with a deuterated form of the biotin linker. Each peptide in the samples is then represented as a pair of peaks in the mass spectrum where the relative peak heights indicate their relative expression levels.

An advantage of the Gygi et al. method, in theory, is that the quantification of the peptides takes place in the MS-mode. When analysing a complex peptide mixture, this means that all of the peptides entering the mass spectrometer at any one point can be quantified simultaneously. This then allows peptides to be selected for further analysis, such as Collision Induced Dissociation (CID) sequencing to identify the protein from which the peptide came, based on whether differential expression occurs. In practice, however, it is not so straightforward e as the only feature identifying peptide pairs from non-peptide ions, noise and contaminating ions is the mass difference between the ICAT-tagged peptides.

Labelling each sample with a different isotope variant of the affinity tag results in an additional peak in the mass spectrum for each peptide in each sample. This means that if two samples are analysed together there will be twice as many peptide peaks in the spectrum. Similarly, if three samples are analysed together, the spectrum will be three times more complex than for one sample alone. It is clear that use of the Gygi et al. approach is limited, since the ever-increasing numbers of peaks will increase the likelihood that two different peptides will have overlapping peaks in the mass spectrum. Different possible charge states of the labelled peptides complicate the analysis further as many different possible mass differences can correspond to labelled peptide pairs. Without any additional means of distinguishing labelled peptide ions from background, it is not feasible to realise the potential of ICAT to identify quantitative differences in the MS-mode.

In practice, most users have ended up “shotgun sequencing” peptides labelled with the ICAT tags and then the quantification is explored retrospectively for the peptides that are identified. While this is a useful method for peptide quantification, it does not achieve the goal of selecting peptides for analysis based on differential expression.

An alternative approach to quantification and identification of peptides is described in WO03/025576 and a related journal publication Thompson et al. (2003, Anal Chem. 75(8): 1895-1904). In this approach, a pair (or more than two) of “mass normalised” tags is coupled to the peptides in a mixture. The tagged peptides have the same overall mass because the tags are designed to have the same mass. The Tandem Mass Tags (TMTs) contain two components, a tag moiety and a mass normaliser moiety. A pair of tags can be constructed such that tag moiety in one of the tags has a different mass from the tag moiety in the other tag but that the overall mass of the mass marker will be the same because the corresponding mass normaliser moieties are designed to balance the overall mass of the mass marker. These two components are separated by a collision cleavable linker that fragments when the peptide is sequenced using CID methods. The cleavage of this linker then releases the tag moieties allowing them to be detected independently amongst the fragments of the labelled peptide. This approach has the advantage that tagged peptides can be sequenced and quantified simultaneously in a shotgun analysis of a sample of peptides. Another version of this technology referred to as “iTRAQ” has also been developed (Ross et al., 2004, Molecular & Cellular Proteomics 3: 1154-1169).

While the isobaric mass tag (or TMT) approach simplifies the process of quantifying peptides in the shotgun method compared to ICAT, there are some limitations. For example, the TMT approach does not enable selection of peptides for sequence analysis based on differential expression. More importantly, it has been reported that relatively high collision energies are needed to get quantitative results from some of the more common classes of peptide ion (Wiese et al., 2007, Proteomics 7(3):340-350). This reduces the reliability of sequencing data. In addition, the tags developed for the TMT method are not stable in water resulting in complicated labelling protocols. Some side reactions have also been reported (see Ross et al. 2004, above) where the tags couple to tyrosine as well as to the intended primary amino groups in peptides.

In summary, the prior art discloses methods or compositions that allow for comparative, quantitative analysis of biomolecule samples by mass spectrometry, and also methods that can improve the signal to noise ratio achievable in mass spectrometry based detection systems for the analysis of biomolecules, particularly peptides. Specifically, the use of isobaric mass tags can provide confirmation that a mass peak in a spectrum was caused by the presence of a mass label. However, isobaric mass tags (or TMTs) have limitations when used for the analysis of peptides and proteins. Since it is the intention that the tag itself is detected, the tags themselves carry a charge or at least are designed so that they ionise easily in a mass spectrometer. The presence of the tag does however alter the behaviour of the analyte being detected by virtue of its charge carrying ability. The tagged peptide may thus have a different charge distribution when compared to the untagged peptide. This may be a more significant problem if the peptide carries more than one tag. In addition, since the tags are designed to localise a charge on themselves, if the tag is coupled to a side-chain such as lysine or cysteine, the presence of the localised charge could change the pattern of fragmentation of the peptide, which pattern is normally used to identify the peptide.

The present invention is directed in part to labelling of analytes such as peptides for detection by neutral loss mass spectrometry and associated methods of analysing tagged analytes such as peptides by neutral loss mass spectrometry.

According to the present invention, there is provided in one aspect a mass marker for labelling of an analyte detectable by mass spectrometry such as neutral loss mass spectroscopy, in which the mass marker comprises a neutral loss mass modifier linked via a first collision cleavable linker to a reactive group having reactive functionality for attachment to the analyte, and in which the neutral loss mass modifier upon cleavage from an during mass spectroscopy is uncharged (neutral).

The invention encompasses in one aspect a neutral loss mass modifier which upon cleavage from the analyte is split into two or more oppositely charged ions which are uncharged in combination. Typically, such oppositely charged ions will attract each other to form an overall uncharged neutral loss mass modifier.

Further aspects and features of the invention are set out in the appended claims and/or elaborated below.

Also provided according to the invention is a neutral loss mass marker having a form
neutral loss mass modifier-collision cleavable linker-reactive functionality
   wherein the neutral loss mass modifier does not localise a charge onto itself during ionisation and mass spectrometry.

In different aspect of the invention, there is provided a set of two or more neutral loss mass markers having a form
neutral loss mass modifier-collision cleavable linker-reactive functionality
   wherein the neutral loss mass modifier does not localise a charge onto itself during ionisation and mass spectrometry.

None of the above-mentioned prior art documents disclosure or suggest the use of neutral loss mass analysis, or mass markers according to the invention, for use in analysing tagged or labelled analytes. Neutral loss mass spectrometry measures the change in mass-to-charge ratio of ions subjected to collisions that result in the loss of neutral fragments. This method of mass spectrometry may be applied in the analysis of mass markers if a marker is used that can be cleaved from an analyte releasing a neutral fragment. If this sort of marker is coupled to its analyte in such a way as to avoid changing significantly the natural ionisation of the analyte, it is possible to obtain the benefits of markers without significantly perturbing the analyte and properties of the analyte in the mass spectrometer.

Moreover, as elaborated below, neutral loss markers enable quantification of complex mixtures in the MS-mode as they can provide a method to confirm the identity of tagged peptides (to which the mass markers are attached), thus distinguishing them from non-peptide material, noise and contamination.

The present invention encompasses mass labels which can be detected in a background of contamination and which enable tagged analyte (i.e. analyte to which the mass marker has been attached) to be identified as being tagged by neutral loss mass spectrometry. The neutral loss mass markers of the invention may be used to label an analyte without significantly altering the charge state of the tagged peptide.

In addition, the invention provides a set (or array) of two or more markers, which allows discrete samples to be labelled and which allow the relative quantities of corresponding components in each sample to be determined.

Furthermore, the invention provides an array of markers, which can be resolved in a compressed mass range so that the markers do not interfere substantially with separation processes such as electrophoresis or chromatographic separations.

Additionally provided by the present invention is an array of neutral loss mass markers, comprising two or more sets of neutral loss mass markers as defined herein.

The invention also provides a method of analysing analytes such as biomolecules (particularly peptides) using neutral loss marker tagged-labels which exploit the labels to maximise throughput, signal to noise ratios and sensitivity of such assays.

Each marker in the set may be chemically identical but the masses of the neutral loss mass modifiers may be altered by isotope substitutions.

The mass marker may comprise an affinity capture ligand or reagent. The affinity capture ligand may for example be biotin.

In another aspect of the invention, there is provided a set of two or more mass markers of the following form
neutral loss mass modifier-collision cleavable linker-neutral mass normaliser-reactive functionality
   wherein the neutral loss mass modifier and the neutral mass normaliser do not localise a charge onto themselves during ionisation and mass spectrometry.

In a further aspect the invention, there is provided a set of two or more mass markers of the following form:
neutral loss mass modifier-collision cleavable linker 1-neutral mass normaliser-collision cleavable linker 2-reactive functionality
   wherein the neutral loss mass modifier and the neutral mass normaliser do not localise a charge onto themselves during ionisation and mass spectrometry, and wherein collision cleavable linker 1 cleaves at a lower collision energy than collision cleavable linker 2.

Where a mass marker according to the present invention comprises a second collision cleavable linker (or collision cleavable linker 2), this second linker may be used to remove the mass marker or the neutral loss mass normaliser component thereof, if the neutral loss mass modifier has already been removed via the first collision cleavable linker (or collision cleavable linker 1) from the labelled analyte.

Where all markers in the set have the same mass and the sum of the masses of the neutral loss mass modifier and the neutral mass normaliser are the same, for each mass marker in the set the mass of the neutral loss mass modifier and the mass of the neutral mass normaliser may be different from other markers in the array.

In embodiments of the invention, each marker in the set may be chemically identical and the masses of the neutral loss mass modifier and the neutral loss mass normaliser may be altered by isotope substitutions.

The invention also provides a method of analysing an analyte such as a biomolecule or a mixture of analytes such as biomolecules, comprising the steps of:
1) reacting the analyte or mixture of analytes with a mass marker as defined herein to form one or more labelled analytes;
2) optionally, separating the one or more labelled analytes (for example, electrophoretically and/or chromatographically);
3) ionising the one or more labelled analytes;
4) selecting ions of a predetermined mass to charge ratio corresponding to the mass to charge ratio of the preferred ions of the one or more labelled analytes in a mass analyser;
5) inducing dissociation of the selected ions by collision to form collision products; and
6) detecting the collision products to identify one or more analyte ions that are generated by neutral loss of the neutral loss mass modifier.

The separation step 2) may be repeated using different separation parameters. For example, step 2) may include a charge base separation and a hydrophobicity separation.

In certain embodiments where the mass markers comprise an affinity tag (also referred to herein as an affinity capture ligand or reagent), the method may comprise a further step of capturing an affinity-tagged labelled analyte(s) such as biomolecule(s) by a counter-ligand to allow labelled analyte(s) to be separated from unlabelled analyte(s). This further step may takes place before the optional step (2) of the method above.

In certain embodiments, step (4) of the method above may be performed in a first mass analyser of a serial instrument. The selected ions may then be channelled into a separate collision cell where in step (5) of the method they may be collided with a gas or a solid surface. The collision products may then be channelled into a further mass analyser of a serial instrument to detect collision products according to step (6) of the method. Typical serial instruments which may be used include triple quadrupole mass spectrometers, tandem sector instruments and quadrupole Time of Flight mass spectrometers.

In certain embodiments, steps (4), (5) and (6) of the method may be performed in the same zone of the mass spectrometer. This may be effected in ion trap mass analysers and Fourier Transform Ion Cyclotron Resonance mass spectrometers, for example.

In further aspects of the present invention, novel amine labelling methods are provided for labelling biomolecules. These methods are based on the use of sequence-specific endoproteases that cleave polypeptides immediately C-terminal to Lysine residues. This results in peptides with an epsilon amino group at the C-terminus of each fragment peptide, except for the C-terminal peptide from the parent polypeptide, which may not have a C-terminal Lysine group. The cleavage reaction also leaves free alpha amino groups in the cleavage peptides, although the original N-terminal alpha-amino group of the parent polypeptide may be naturally blocked. This means that there are free amino groups at both ends of the majority of the cleavage fragments that would be generated by Lys-C cleavage, which can be easily labelled. The use of Lys-C in combination with amino labelling is an effective method for controlling the number of tags introduced into peptides for subsequent analysis.

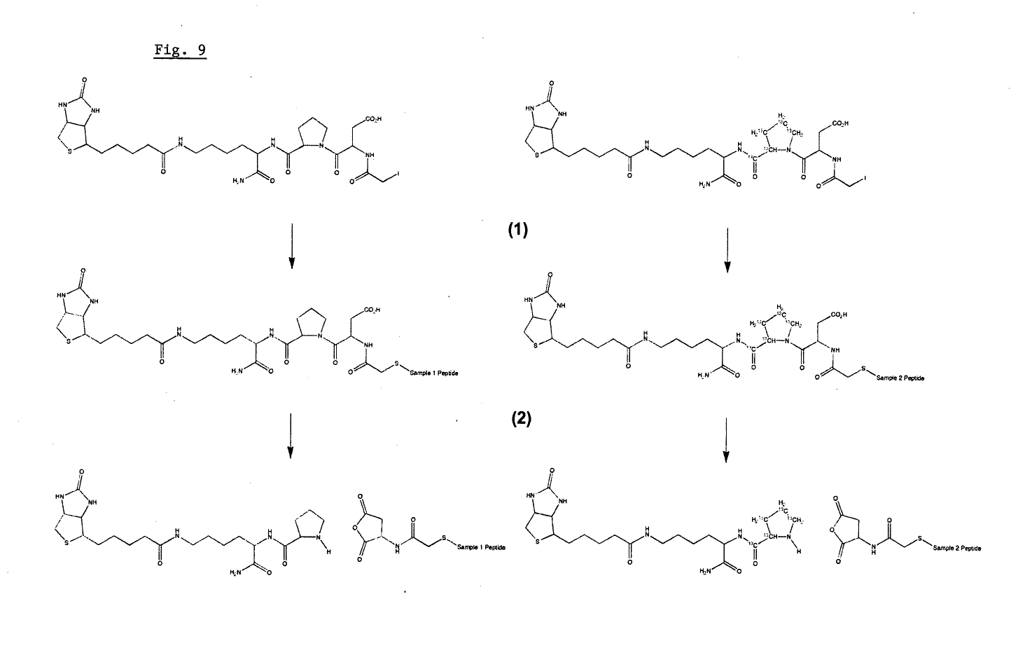

In addition, the Lys-C fragments that are generated from the cleavage of larger polypeptides exist as two distinct populations: those fragments that contain arginine and those fragments that have no arginine. If these Lys-C fragments are labelled with an amino-reactive tag and then cleaved with Arg-C or trypsin, a new fragment population is generated providing additional methods for analysing peptide mixtures. A number of distinct classes of peptides will result from this process of initial cleavage of a polypeptide mixture by Lys-C, amine-labelling with a tag and second cleavage with trypsin or Arg-C (described further below and illustrated in FIGS. 10a and 10b. Different classes of peptides according to the invention are numbered in FIG. 10b).

The term “mass marker” as used herein means a marker (or “tag” or “label”; see below) that is used in mass spectrometry. A mass marker may be detectable by mass spectrometry on its own and/or in combination with an analyte to which it is attached. A mass marker that is cleavable from an analyte to release a neutral fragment is a neutral loss mass marker. The neutral loss mass marker may be coupled or linked to the analyte in such a way as to avoid (significantly) altering ionisation of the analyte during mass spectrometry. In this way, it is possible to obtain the benefits of a mass marker without (significantly) perturbing the analyte and/or properties of the analyte in a mass spectrometer.

Neutral loss mass spectrometry refers a form of mass spectrometry which detects or measures a change in mass-to-charge ratio of ions subjected to collisions that result in the loss of one or more neutral fragments. Thus, ions which differ from each other by a certain number of mass units (equivalent to the lost neutral fragment or fragments) are detected. As elaborated herein, a tandem mass spectrometry device may be used, with a second analyser detecting only those product ions which have dissociated from precursor ions (such as those resulting from ionisation of a labelled analyte) transmitted through a first analyser by a specified neutral mass.

The term “neutral loss mass modifier” (or “mass modifier”) as used herein refers to a molecule or fragment which is releasable attachable to an analyte and which upon cleavage during mass spectrometry releases as a neutral (uncharged) molecule or fragment. The mass modifier has a unique mass. The mass modifier is present in the mass marker to ensure that the mass marker has a desired aggregate mass (for example, in combination with a neutral loss mass normaliser). The mass modifier per se is typically not detected by mass spectrometry due to its lack of charge, but is detectable by mass spectrometry when attached to a charge-bearing analyte.

As used herein, the term “neutral loss marker” is synonymous and used interchangeably with the term “neutral loss mass marker”. The term “neutral loss mass marker” may be used herein in the abbreviated forms “mass marker” or simply “marker”. Additionally, the terms “marker”, “tag” and “label” are synonymous and used interchangeably herein with reference to the invention. For example, a “neutral loss mass marker” is synonymous and used interchangeably with a “neutral loss mass tag”, and a “neutral loss marker” is synonymous and used interchangeably with a “neutral loss tag”. Similarly, the term “neutral loss mass modifier” is synonymous and used interchangeably with the term “neutral mass modifier”.

As used herein, the term “mass marker precursor” refers to a molecule used to form a mass marker of the invention. The terms “neutral loss mass modifier precursor” and “reactive group precursor” refer to a molecule used to form the neutral loss mass modifier or the reactive group, respectively, of a mass marker according to the invention.

Neutral loss mass markers of the invention are suitable for labelling of analytes such as biomolecules including peptides and oligonucleotides (such as for example amino-derivatised oligonucleotides). The term “peptide” encompasses polymers of linked amino acids as well as peptidomimetics comprising, for example, non-natural amino acids and/or modified amino acids and/or modified backbones. Unless otherwise indicated by context, the term peptide when used herein may also refer generically to other suitable analytes susceptible to analysis using a neutral loss mass marker of the invention. A peptide for analysis according to the invention may, for example, have a mass of up to 2,500 Daltons.

As used herein, the term “low collision energy” is a relatively one which differs depending on the mass spectrometer used. The collision cleavable linkers of the present invention may be cleavable at a collision energy below which the majority of (i.e. more than 50% of) or substantial b and y fragments are cleaved from an amide backbone of a peptide.

The term “MS-mode” is understood in the art and typically means a setting whereby ions or fragments produced by CID are allowed to pass to the detector of a mass spectrometer, i.e. without being subjected to further selection and/or manipulation. In tandem and other variations of MS, the MS-mode may be used in combination with other modes of analysis.

The term “aliphatic amino acid” as used herein refers to any one of the group consisting of glycine (G), alanine (A), valine (V), leucine (L), isoleucine (I) and proline (P).

The invention will now be described in further detail by way of example only, with reference to the accompanying drawings, in which:

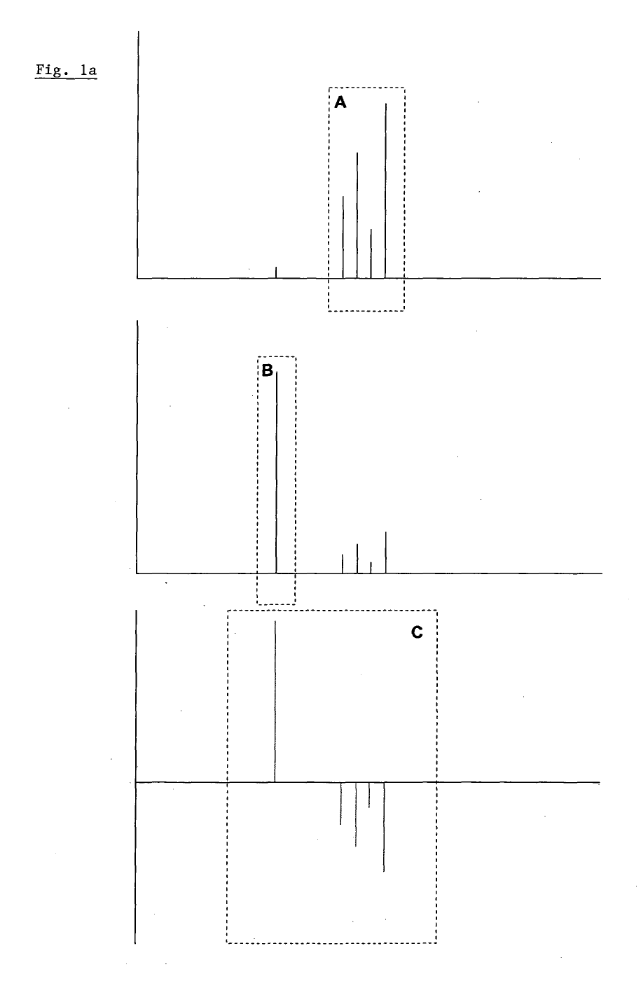

FIG. 1 is an illustration of the use of “difference spectra” to confirm the identity of peptide ions using non-isobaric neutral loss mass markers. In each spectrum shown, the y-axis represents intensity and the x-axis mass-to-charge ratio (m/z);

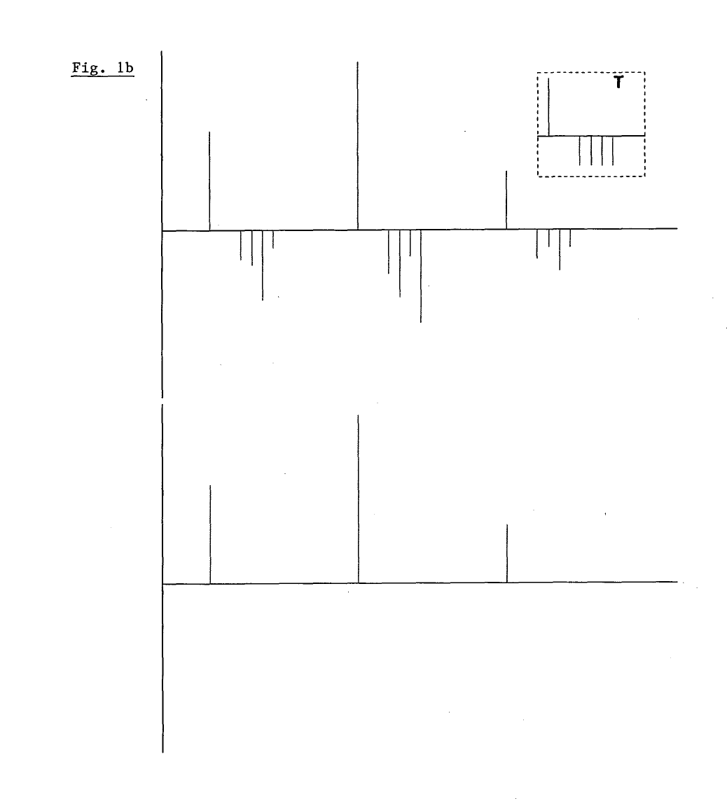

FIG. 2 is an illustration of the use of “difference spectra” to confirm the identity of peptide ions using isobaric neutral loss mass markers. In each spectrum shown, the y-axis represents intensity and the x-axis mass-to-charge ratio (m/z);

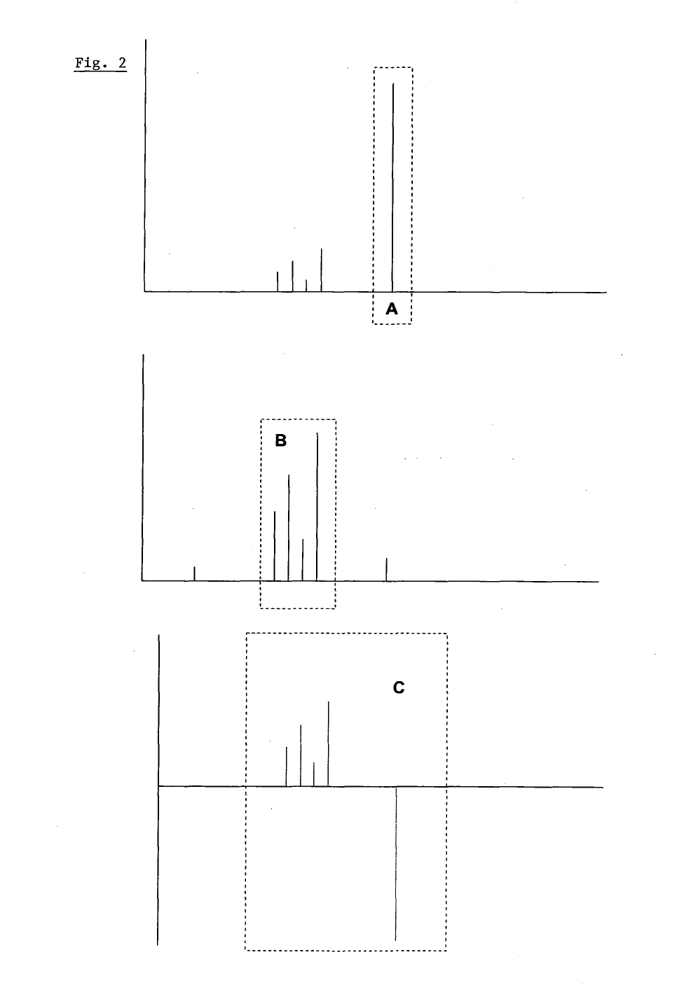

FIG. 3 is a flowchart of the difference spectrum analysis process;

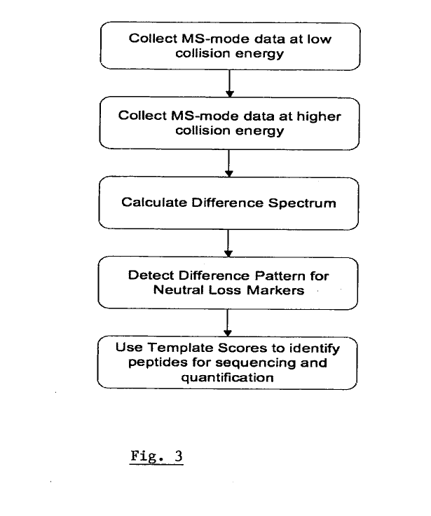

FIG. 4 shows a set of pair of peptides labelled with isotopes of maleic anhydride;

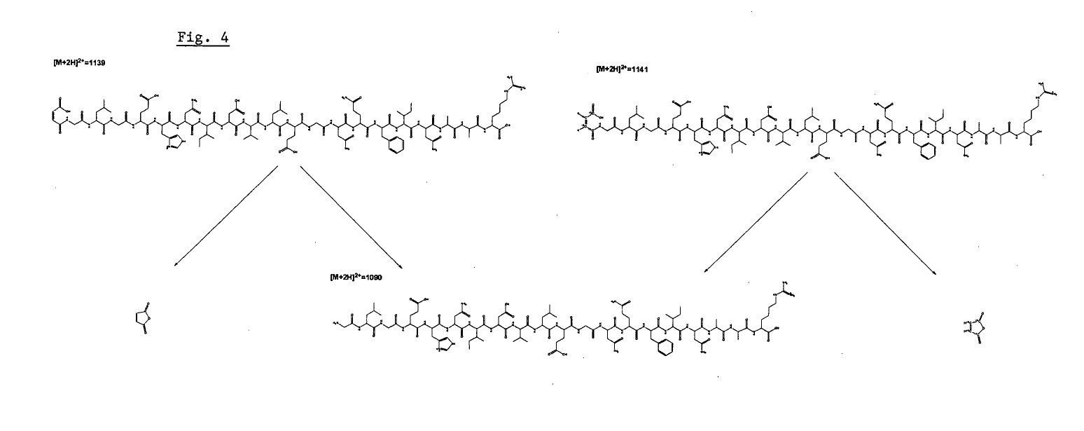

FIG. 5a shows an aldehyde activated mass marker with an aspartic acid-proline tag;

FIG. 5b shows an aldehyde activated mass marker with an alanine-proline tag;

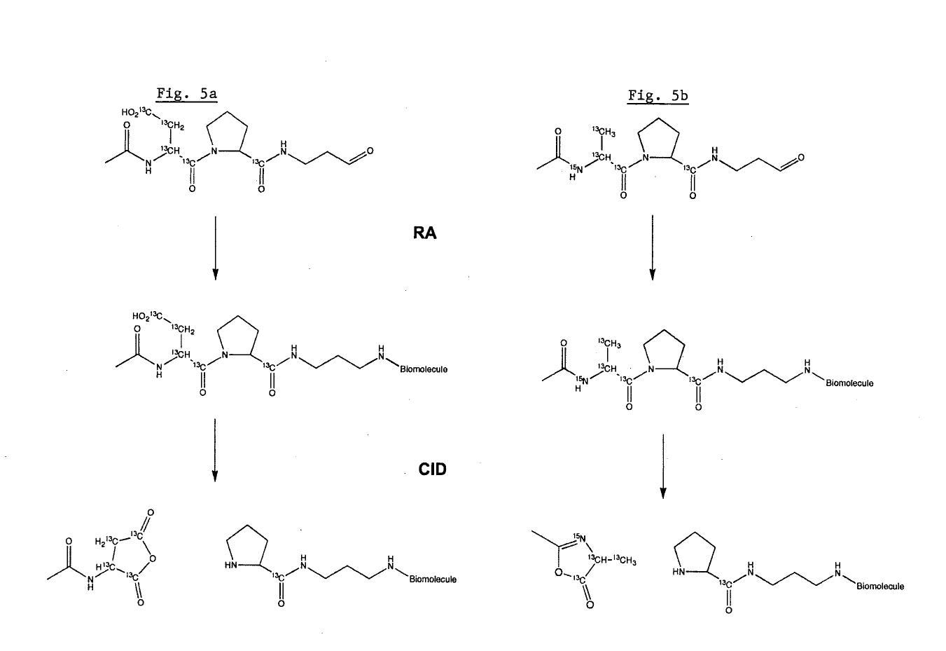

FIG. 6a illustrates 2-methoxy-4,5-dihydro-1H-imidazole activated mass marker with an aspartic acid-proline tag;

FIG. 6b illustrates a neutral loss mass marker activated with an O-methylisourea derivative with an aspartic acid-proline marker;

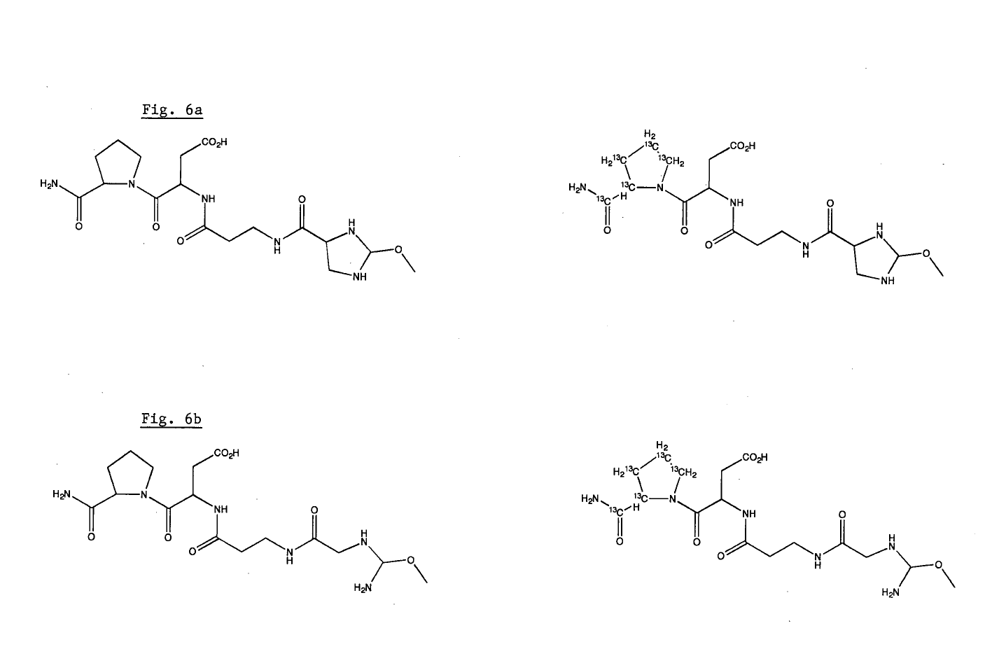

FIG. 7 illustrates a propenyl sulphone activated mass marker with an aspartic acid-proline tag;

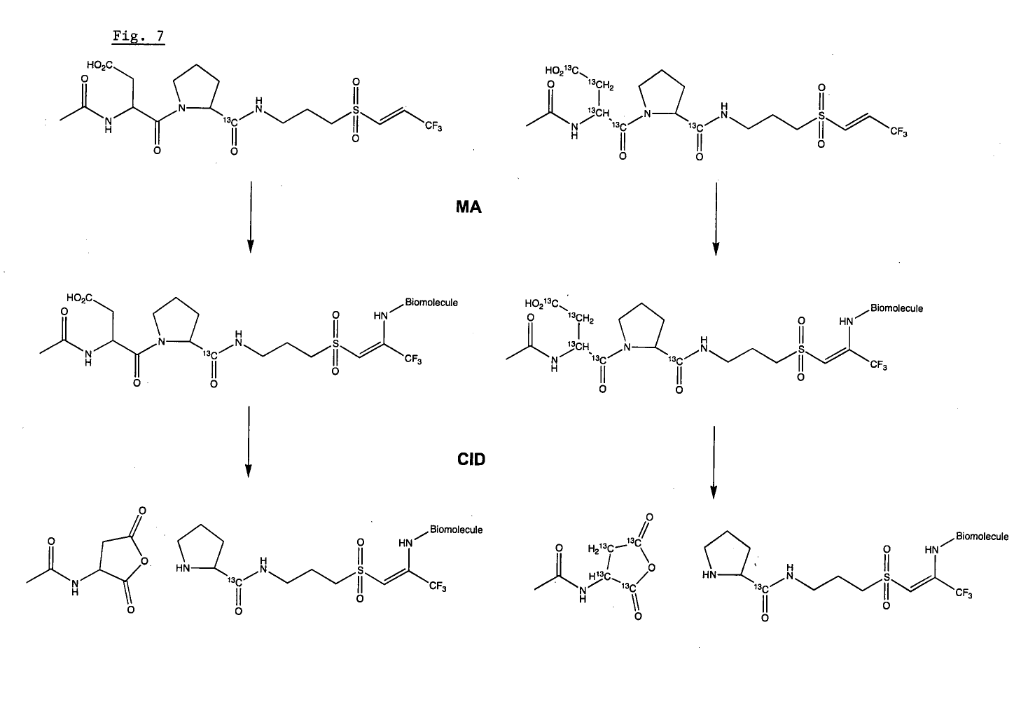

FIG. 8 illustrates a pair of isobaric thiol reactive tags for the labelling of cysteine residues;

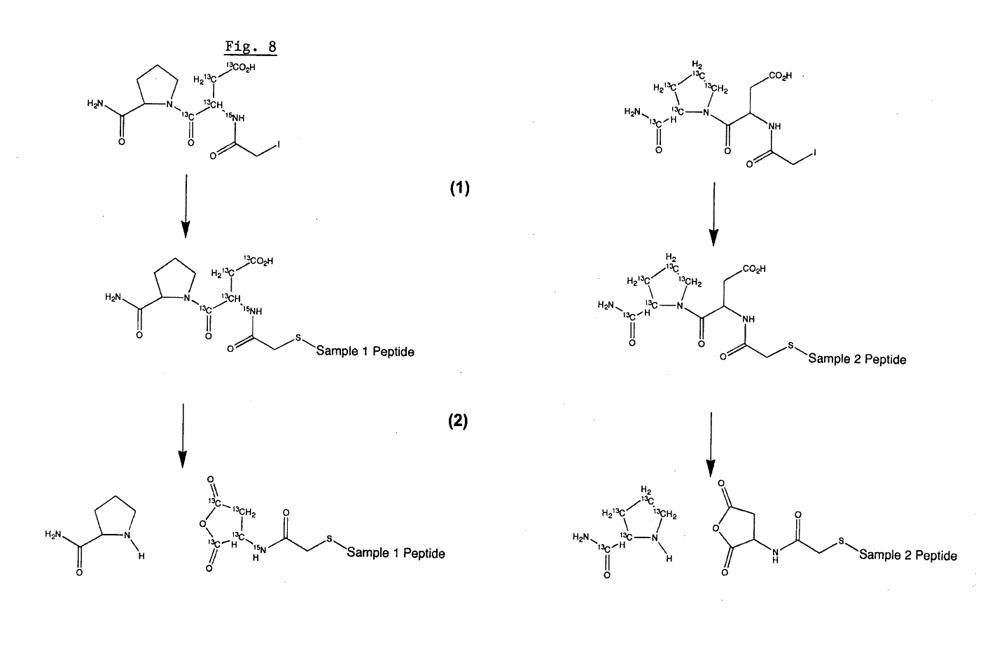

FIG. 9 shows a pair of affinity ligand mass markers with an iodoacetyl functionality for labelling thiols such as cysteine residues;

FIG. 10 shows the first part (a) and second part (b) of a protocol for labelling of peptides that have been cleaved with Lys-C where the labelled peptides are additionally cleaved with trypsin or Arg-C giving various different classes of labelled peptide products;

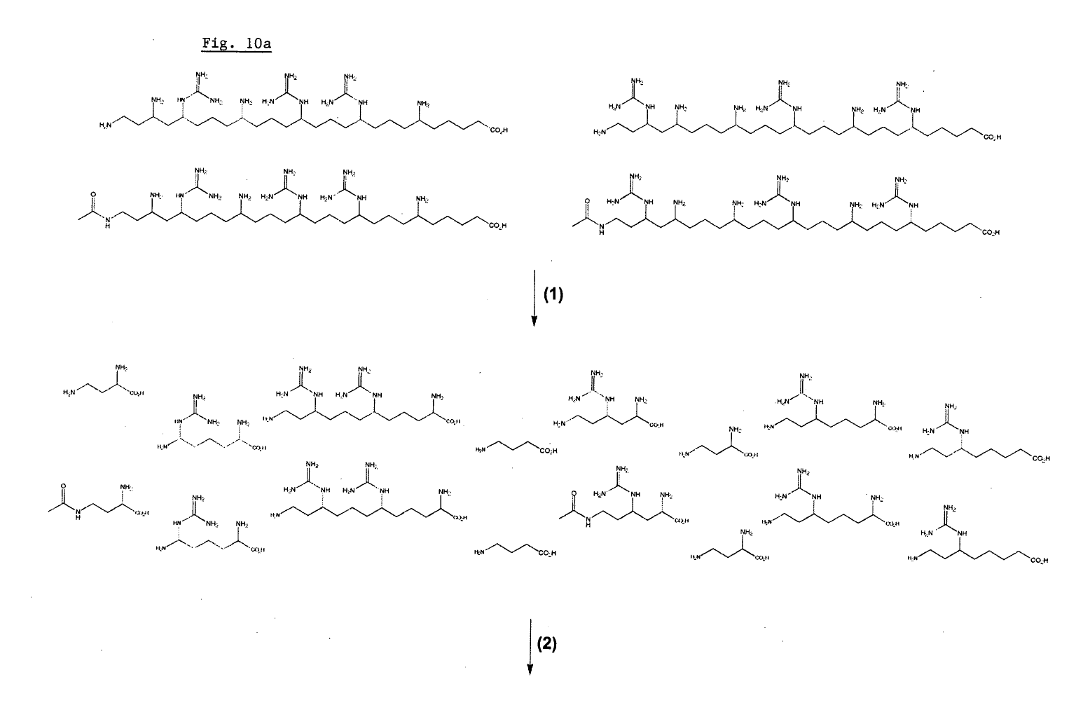

FIG. 11 shows a set of two neutral loss mass markers with an hydrazide functionality for labelling carbohydrates;

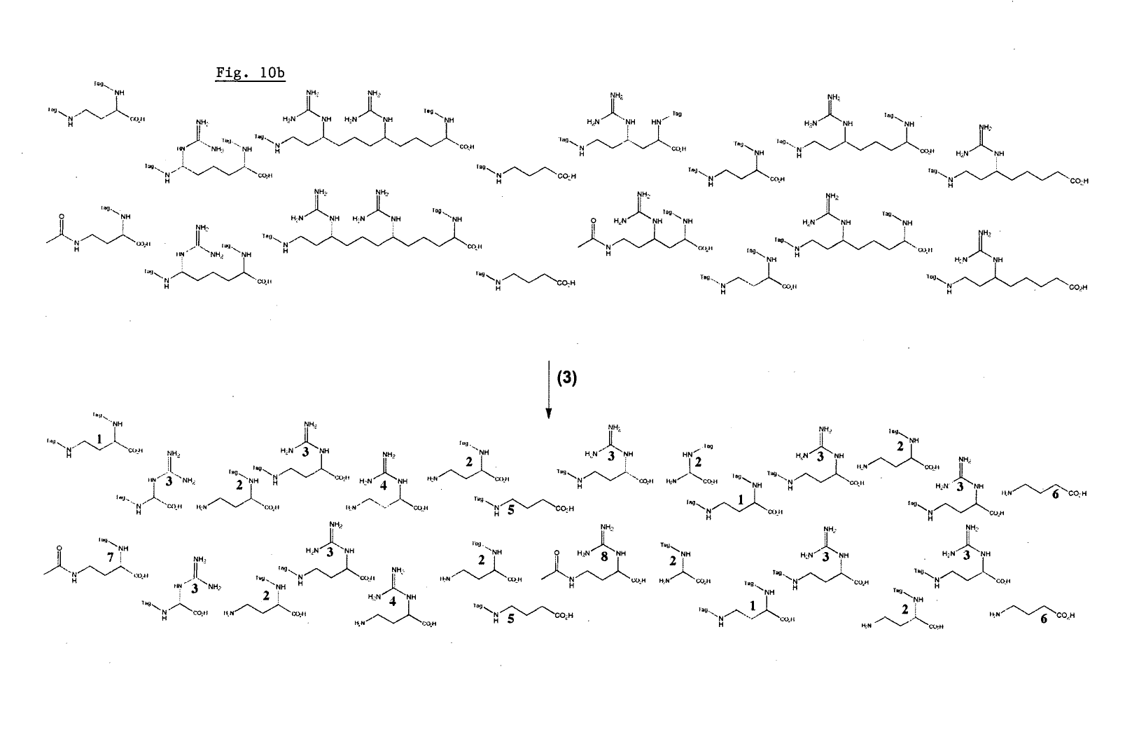

FIG. 12 shows a set of two neutral loss mass markers with a thiol functionality for labelling dehydroalanine and methyldehydroalanine residues;

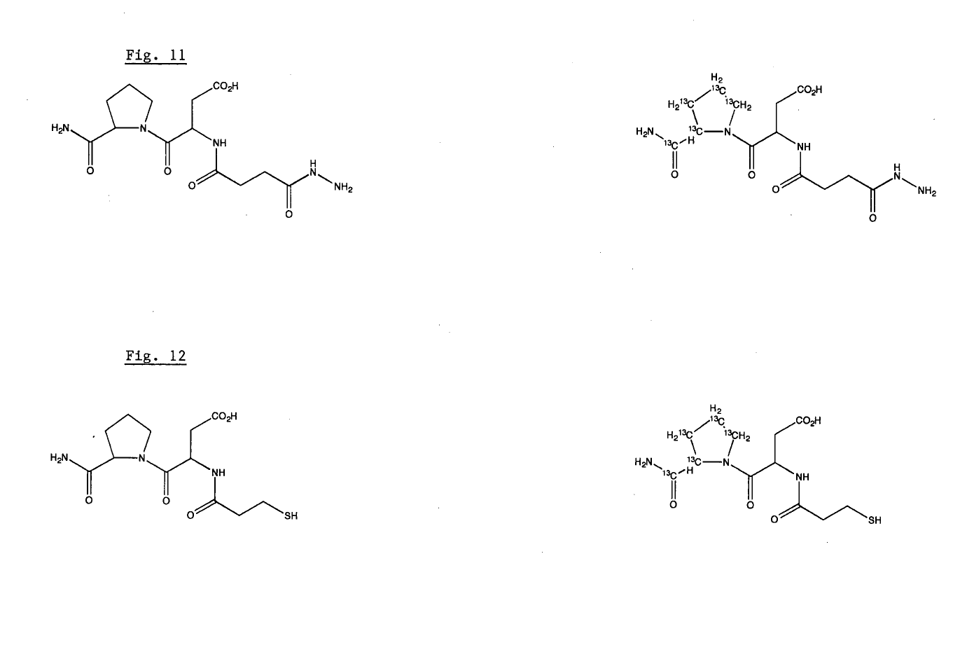

FIG. 13 shows a pair of tags activated with an N-hydroxysuccinimide active ester;

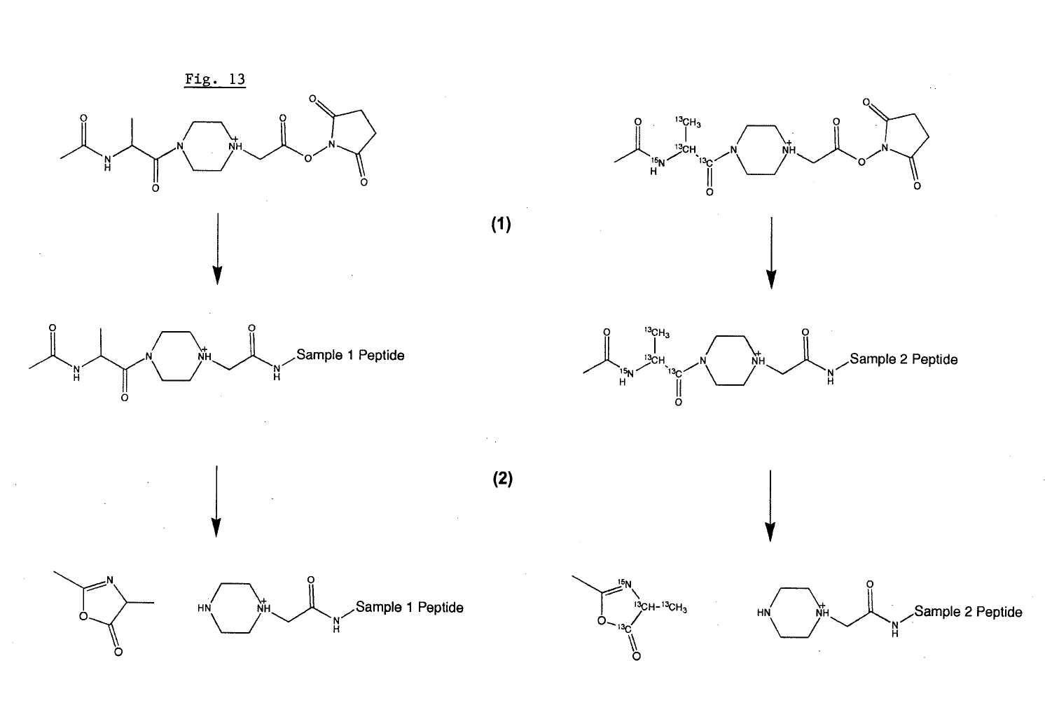

FIG. 14 illustrates a pair of isobaric tags;

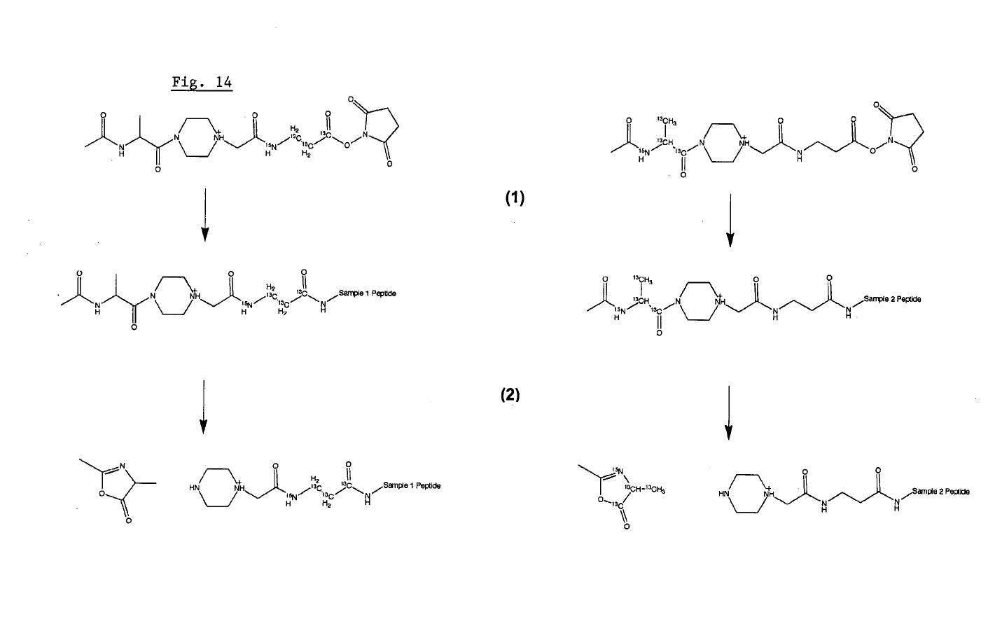

FIG. 15 shows an expected mass-to-charge ratios of the y-series from MS/MS sequencing of the neutral loss tag peptide pair of FIG. 14 coupled to a short peptide. The isobaric parent ions [M+H]⁺=1065.57;

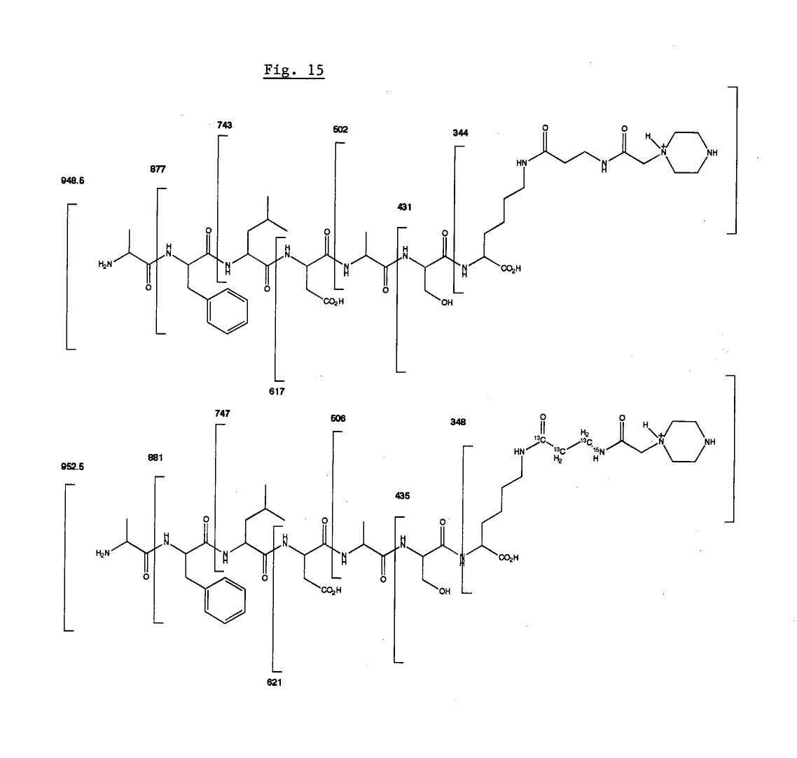

FIGS. 16a and b are schematics showing alternative protocols for the production of isourea reagents;

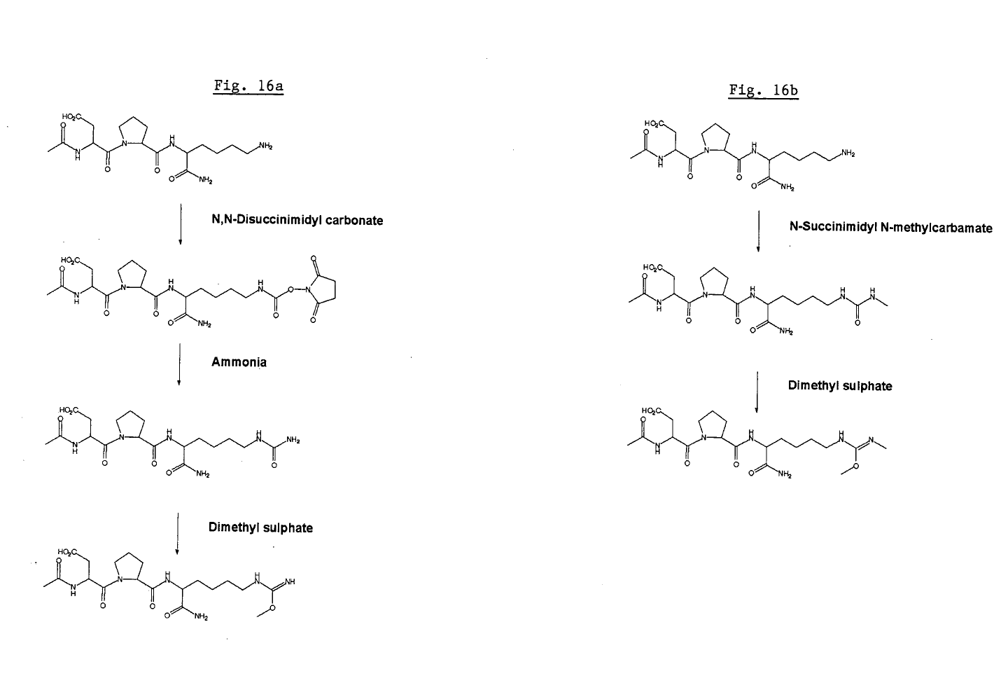

FIG. 17 shows an azide modified neutral loss tag reagent (part 1) and a synthetic protocol for the production of a propynyl isourea reagent (part 2);

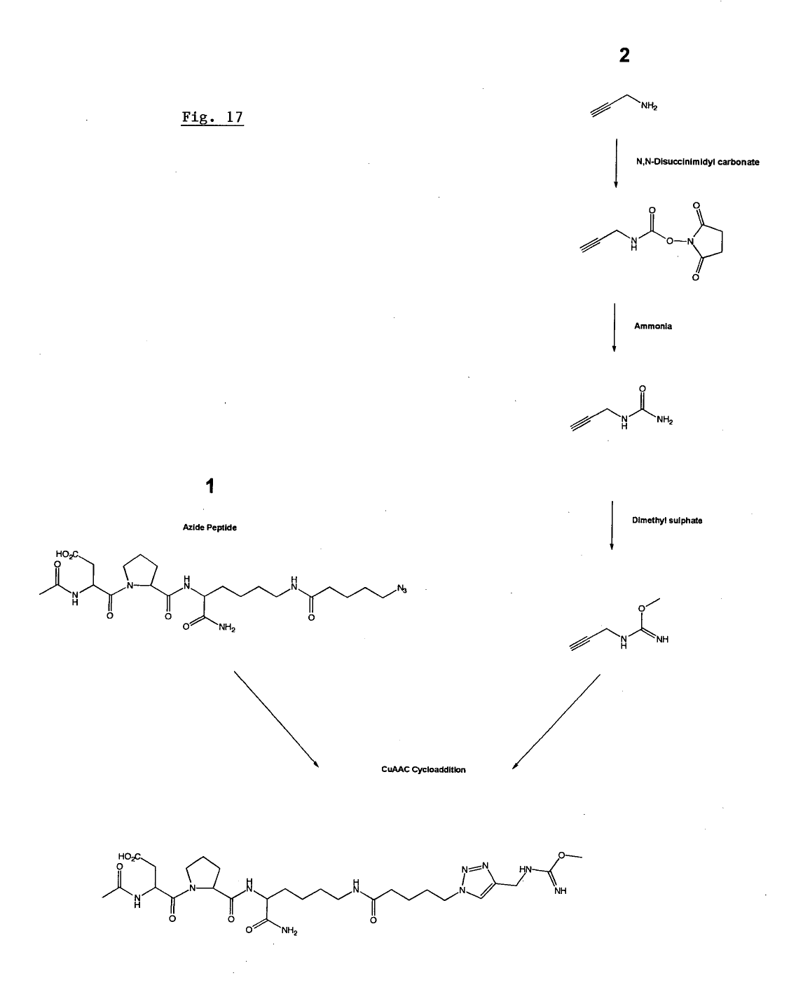

FIG. 18 shows in part 1 the azide modified neutral loss tag reagent as in part 1 of FIG. 17 and a schematic of a synthetic protocol for the production of a propyne-linked isourea reagent (part 2);

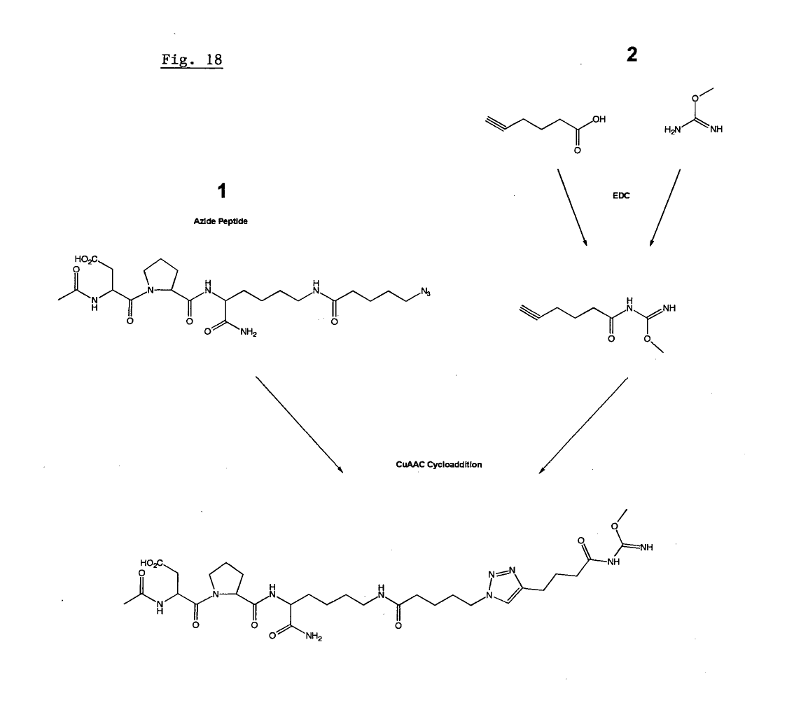

FIG. 19 shows a synthetic protocol for the production of aldehyde reagents;

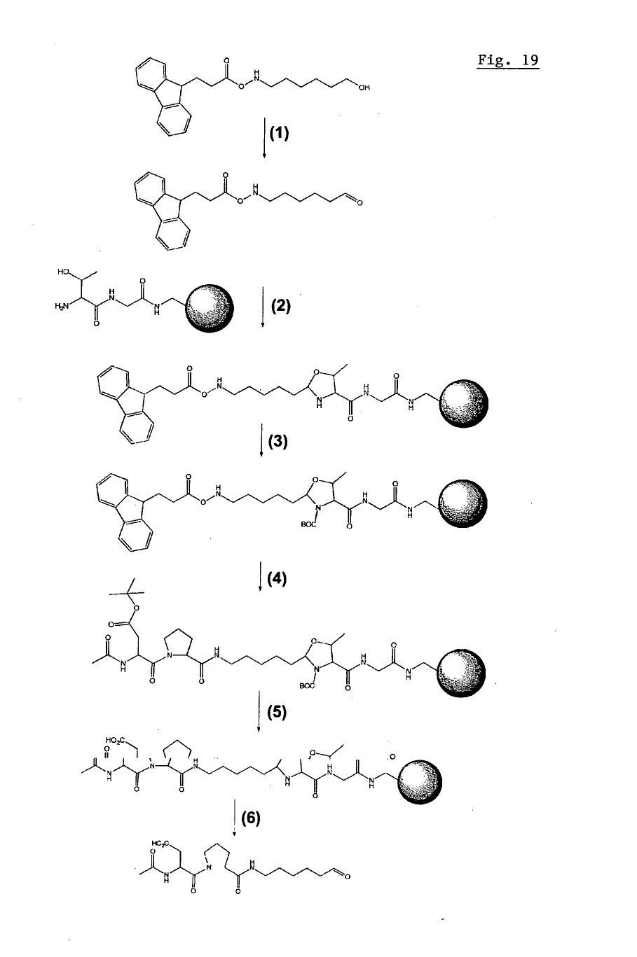

FIG. 20 shows the expected fragmentation of a peptide “SmallAspPip” [SEQ ID NO: 1] to give the corresponding neutral loss daughter ion;

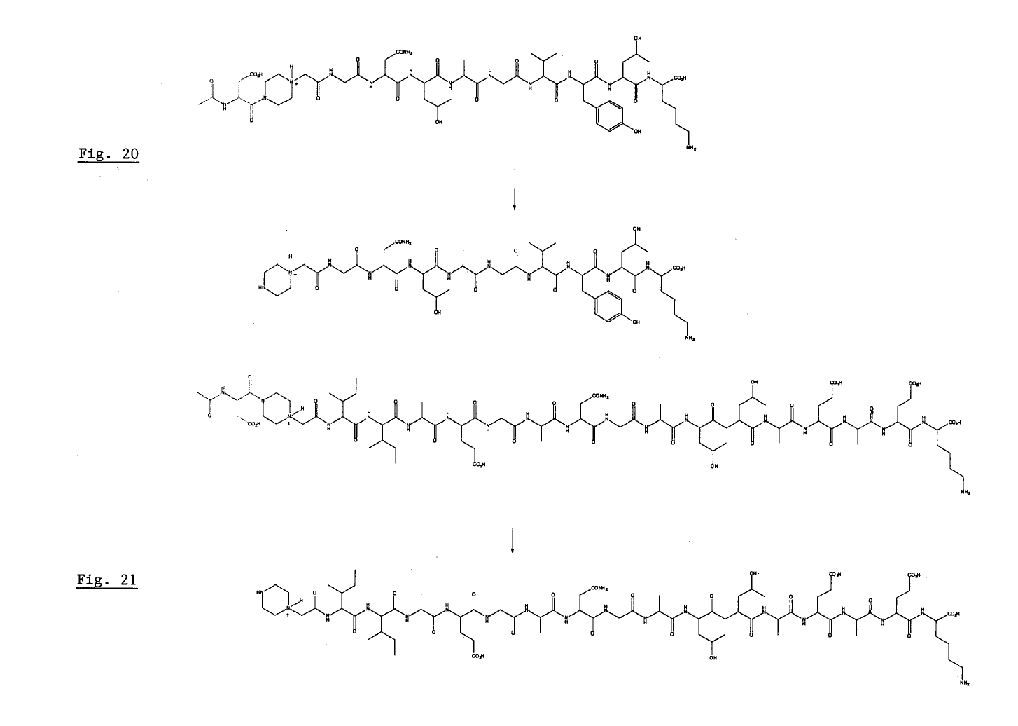

FIG. 21 shows the expected fragmentation of a peptide “MediumAspPip” [SEQ ID NO: 2] to give the corresponding neutral loss daughter ion;

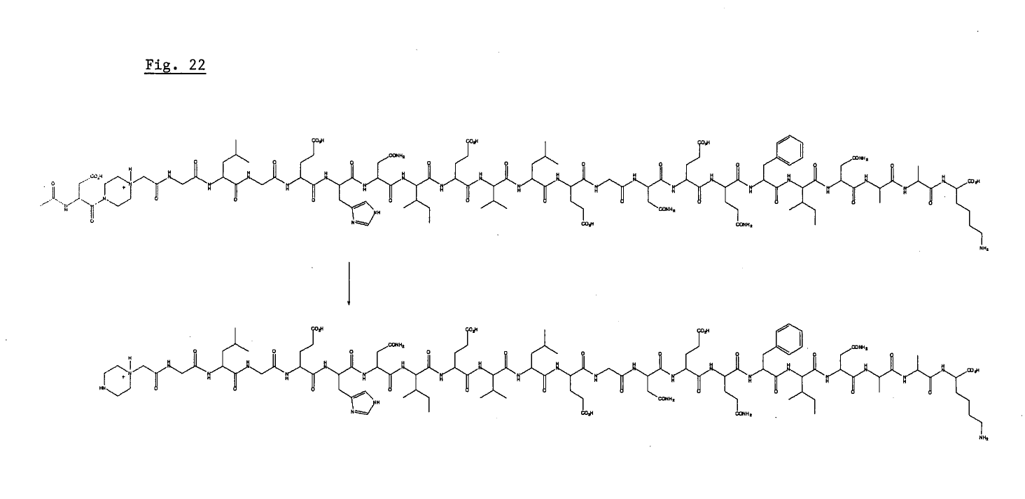

FIG. 22 shows the expected fragmentation of a peptide “LargeAspPip” [SEQ ID NO: 3] to give the corresponding neutral loss daughter ion;

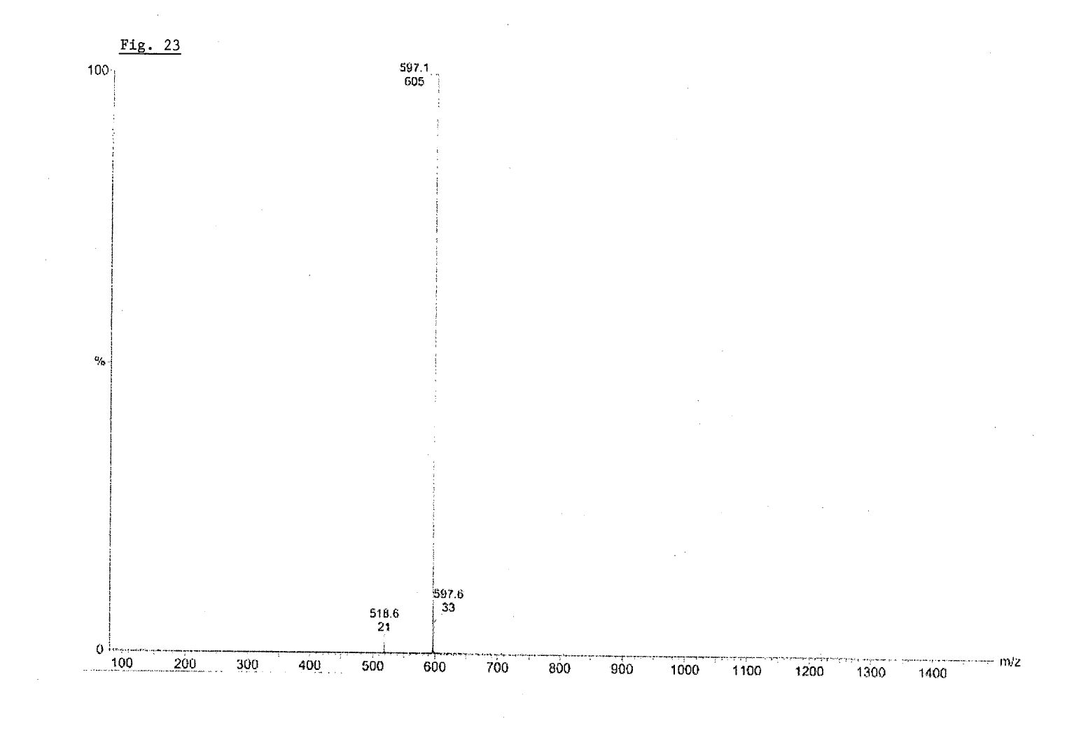

FIG. 23 shows ESI-MS spectra of the peptide SmallAspPip [SEQ ID NO: 1] where the CID voltage in the collision cell has been set to 10V;

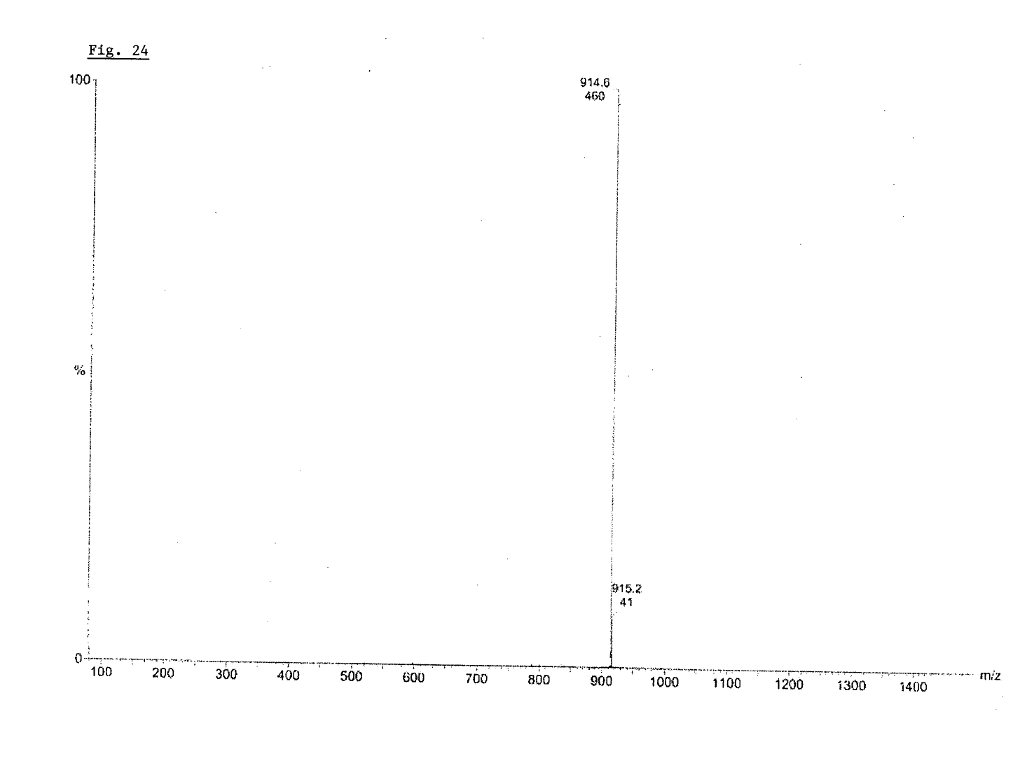

FIG. 24 shows ESI-MS spectra of the peptide MediumAspPip [SEQ ID NO: 2] where the CID voltage in the collision cell has been set to 10V;

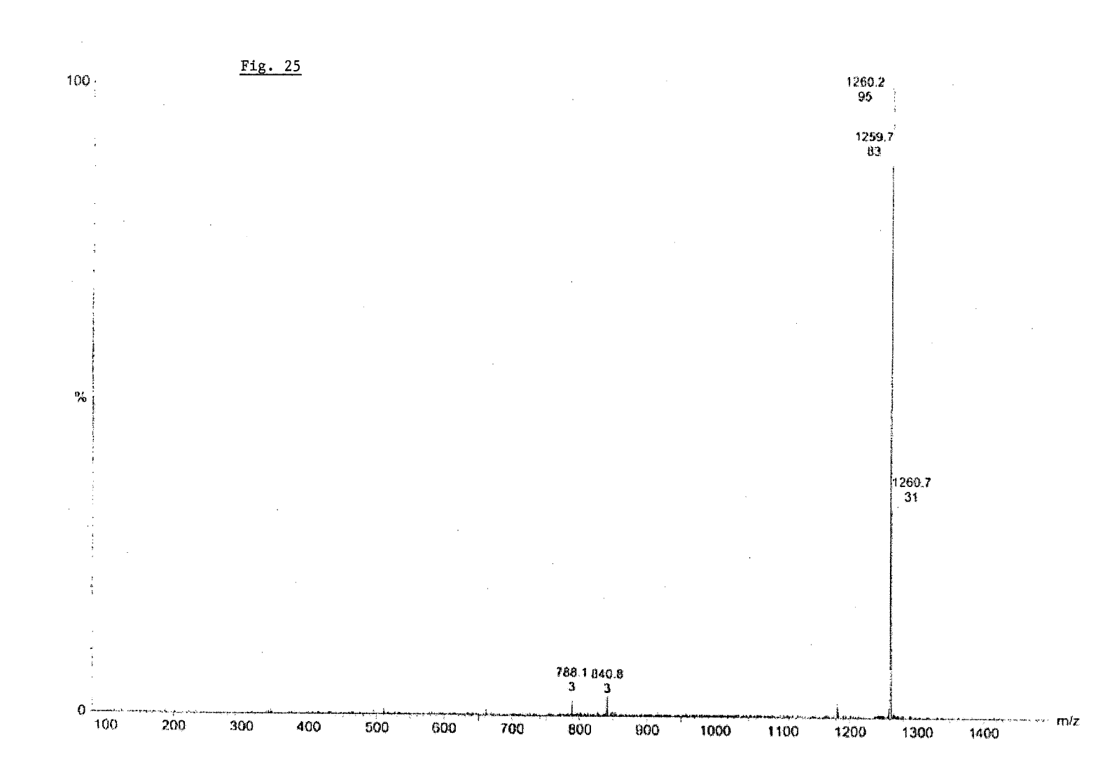

FIG. 25 shows ESI-MS spectra of the peptide LargeAspPip [SEQ ID NO: 3] where the CID voltage in the collision cell has been set to 10V;

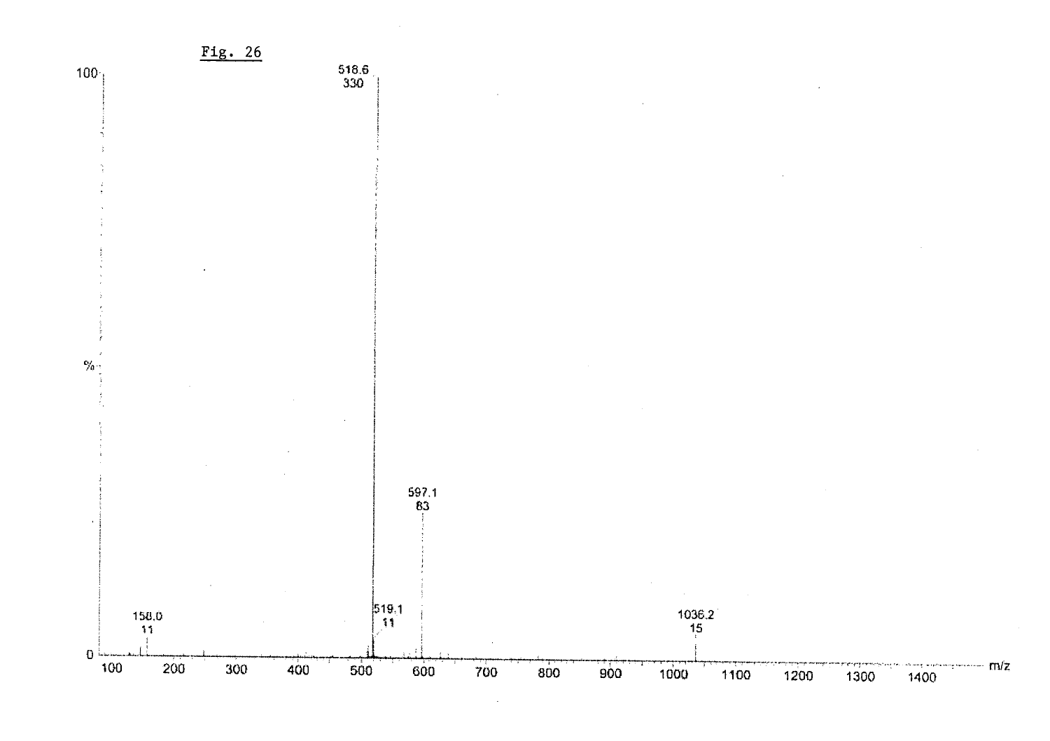

FIG. 26 shows ESI-MS spectra of the peptide SmallAspPip [SEQ ID NO: 1] where the CID voltage in the collision cell has been set to 20V;

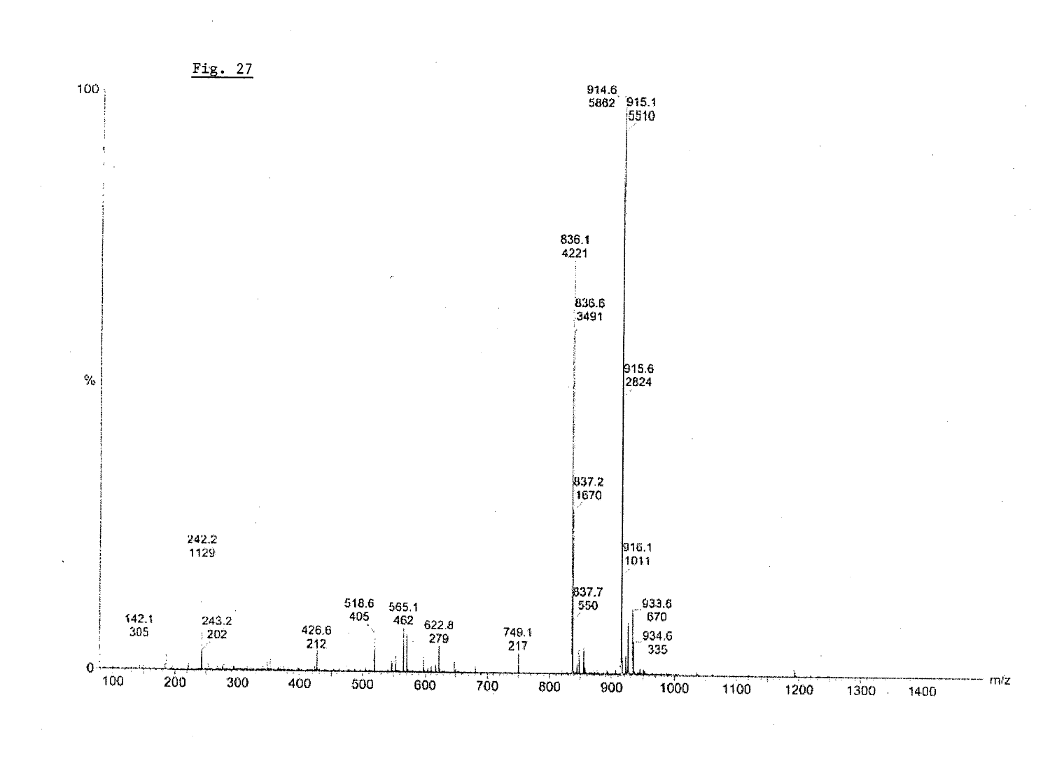

FIG. 27 shows ESI-MS spectra of the peptide MediumAspPip [SEQ ID NO: 2] where the CID voltage in the collision cell has been set to 20V;

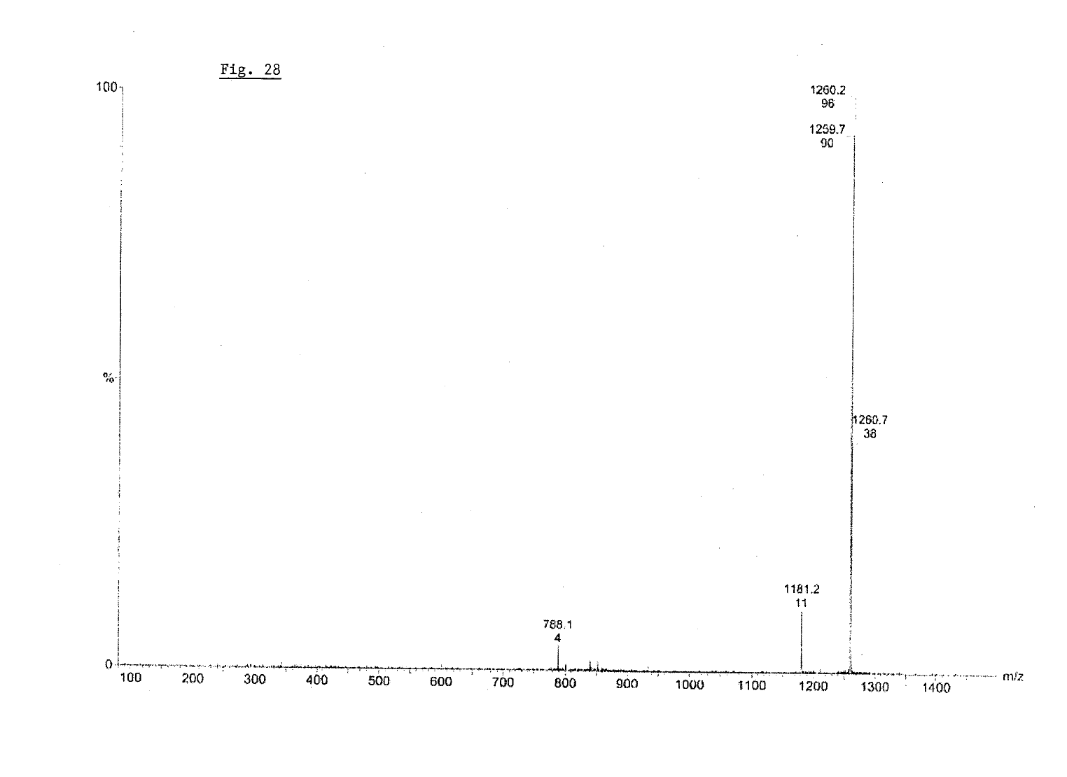

FIG. 28 shows ESI-MS spectra of the peptide LargeAspPip [SEQ ID NO: 3] where the CID voltage in the collision cell has been set to 30V;

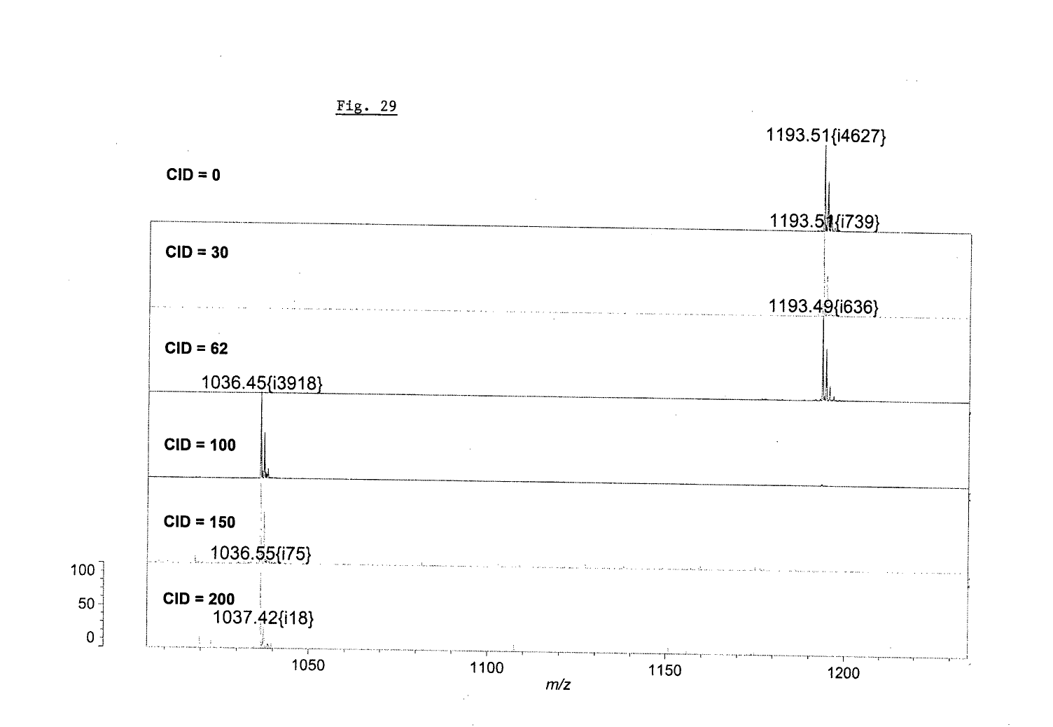

FIG. 29 shows MALDI-MS/MS spectra of the peptide SmallAspPip [SEQ ID NO: 1];

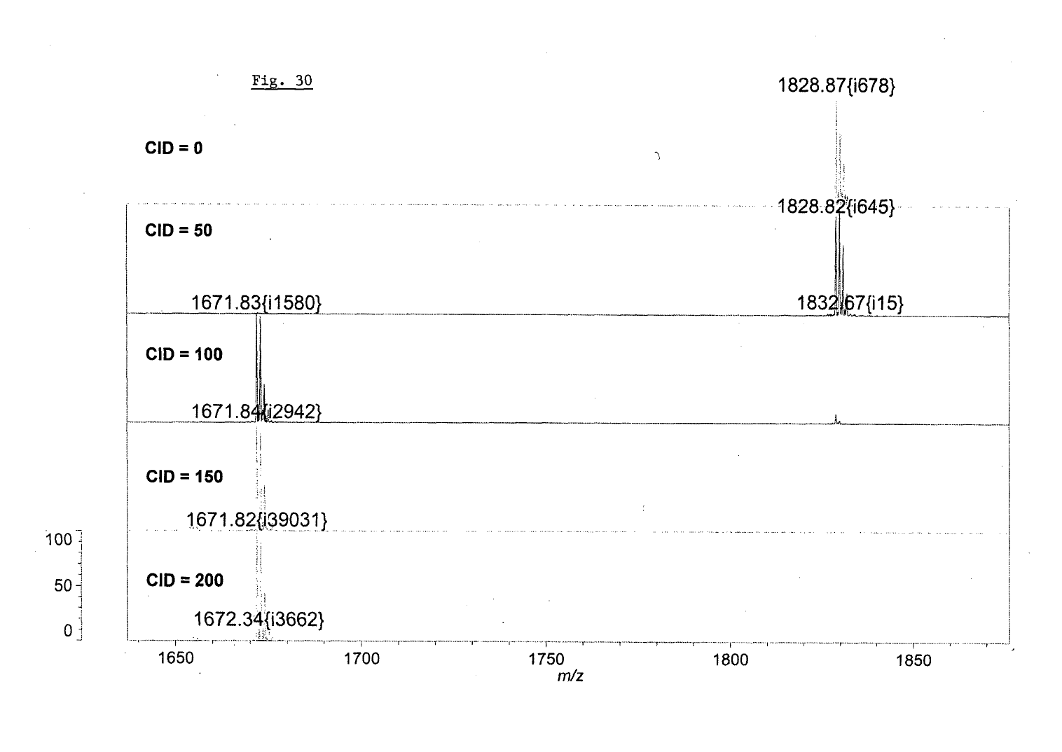

FIG. 30 shows MALDI-MS/MS spectra of the peptide MediumAspPip [SEQ ID NO: 2];

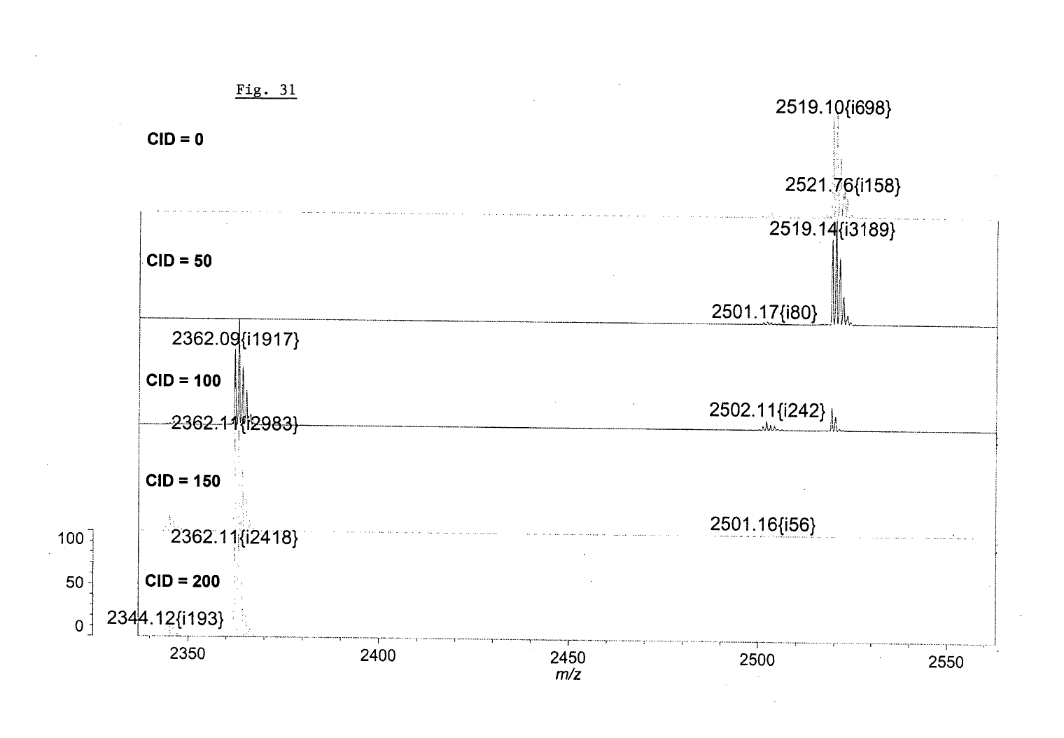

FIG. 31 shows MALDI-MS/MS spectra of the peptide LargeAspPip [SEQ ID NO: 3];

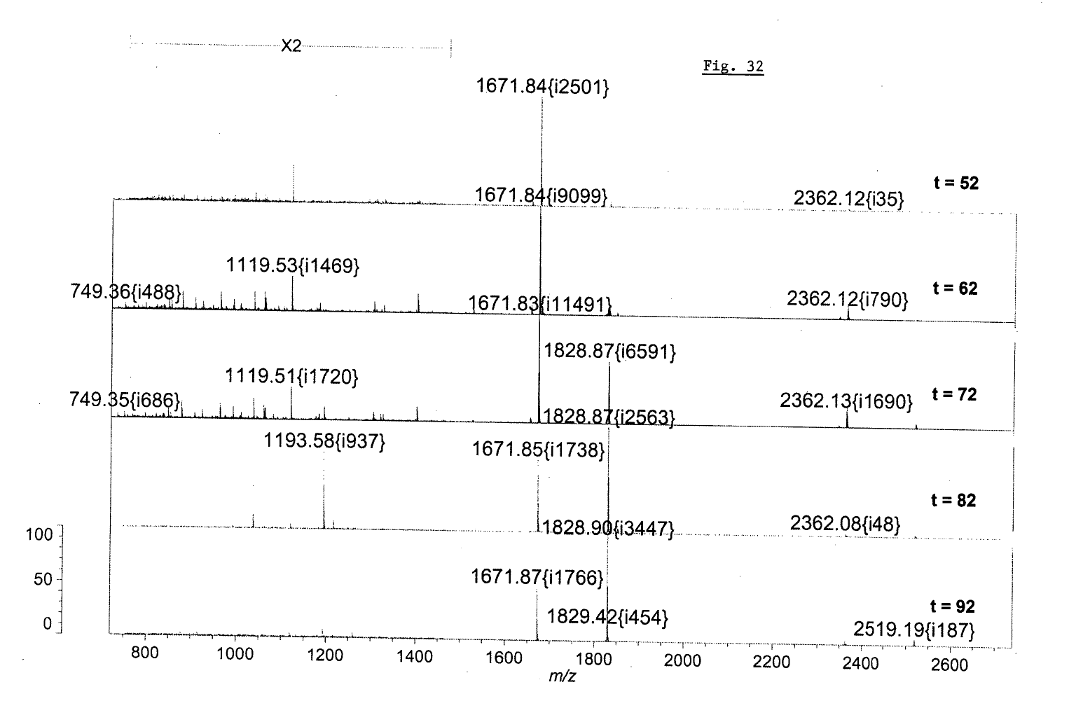

FIG. 32 shows MALDI MS/MS spectra of a mixture of the three peptides SmallAspPip [SEQ ID NO: 1], MediumAspPip [SEQ ID NO: 2] and LargeAspPip [SEQ ID NO: 3].

Tagging of analytes such as peptides for analysis by mass spectrometry is well known in the art, but until now the approach taken has been either to introduce a neutral loss mass modifier tag into the peptide and detect the mass shifted peptide, i.e. the tag and the peptide is detected, or in the alternative, the tag is linked to a biomolecule and is designed to carry a charge and be cleaved from the peptide. In this second approach, only the tag is detected. This invention proposes an alternative strategy, in which a cleavable but non-ionising or charge bearing mass marker is used to label an analyte such as a biomolecule, particularly peptides and oligonucleotides. The presence of the marker is inferred from analysis of neutral loss processes in which the marker is cleaved from the analyte (such as peptide) by collision, i.e. mass differences are used to detect labelled analytes such as biomolecules.

The principle behind the invention can be explained as follows. Consider a peptide with a mass of 500 Daltons. A cleavable neutral loss tag or marker of 100 Daltons can be coupled to this peptide. If the peptide forms a singly protonated and singly charged ion, it will have a mass-to-charge ratio of 601. The presence of this ion in a mass spectrum is indicative of the presence of the peptide but without any additional information it is difficult to determine whether the ion at m/z 601 is really the labelled peptide. However, since the tag can cleave upon collision induced dissociation at a low energy, i.e. at an energy which leaves the peptide most intact, the identity of the peptide can be confirmed by increasing the collision energy until the neutral loss tag is cleaved from the peptide producing a characteristic shift in mass-to-charge ratio to 501 (the protonated form of the native peptide). The use of a tag that can cleave by neutral loss to produce characteristic changes in the mass-to-charge ratio of a labelled biomolecule after changes in collision energy is thus very useful to confirm the presence and identity of the labelled biomolecule.

In addition, two or more identical biomolecules from different samples can be labelled with different isotopes of a neutral loss marker. The differently labelled forms of the biomolecule from different samples will appear as different peaks in the mass spectrum, e.g. if a pair of isotopic tags with masses of 100 and 110 are used to label a 500 Dalton peptide from two different samples, two peaks would appear at m/z 601 and 611 if the labelled peptides form singly protonated and singly charged ions. The intensities of these isotopic tag peptide conjugates will reflect the relative quantities of the peptides in their source samples giving quantitative information about the peptides. The identities of these ions can then be confirmed by increasing the collision energy to cleave the neutral loss tags. The 601 and 611 ions will then decrease in intensity as the collision energy is increased and the 501 ion will increase in intensity in a characteristic fashion to confirm the identities of the peptides.

Exemplar compounds that behave as neutral loss mass markers are described further below, together with useful methods for analysis of analytes such as biomolecules labelled with such mass markers.

### Neutral Loss Analysis of Labelled Biomolecules by Mass Spectrometry

Typically, neutral losses are detected by a ‘Neutral Loss Scan’ on an instrument like a triple quadrupole mass spectrometer. However, it is also possible and advantageous to use other instruments such as ion traps, Time of Flight and quadrupole Time of Flight instruments to detect neutral losses. Hybrid instruments with the properties of both a triple quadrupole and an ion trap such as the Q-TRAP instrument (MDS Sciex, Toronto, Ontario, Canada) may be used. Hybrid TOF instruments are particularly useful.

In a neutral loss scan on a triple quadrupole or tandem quadrupole instrument, the first quadrupole scans over a pre-determined mass range, the selected ions are then subjected to collision in the collision cell of the instrument and the expected neutral loss is detected by scanning the final quadrupole in step with the first quadrupole but with the final quadrupole set to only allow passage of ions with the shift in mass-to-charge ratio that would be caused by the expected neutral loss. For example, in a scan for a neutral loss of 15 Daltons from ions in the +1 charge state, the final quadrople will scan in step with the first quadrupole but with a difference of 15 Daltons in the mass of the ions that it will allow to pass through. Thus, to detect an ion in the +1 charge state with a mass-to-charge ratio of 100 that can undergo this neutral loss, the first quadrupole will be set to allow ions with a m/z of 100 to pass, and the final quadrupole will be set to 85. Similarly, to detect an ion in the +1 charge state with a mass-to-charge ratio of 200 that can undergo this neutral loss, the first quadrupole will be set to allow ions with a m/z of 200 to pass, and the final quadrupole will be set to 185, etc.

To allow detection of labelled analytes such as peptides and other biomolecules in a neutral loss scan, the invention provides a marker that can undergo neutral loss at relatively low collision energies. This means that peptides can be tagged with the neutral loss markers of the invention and when analysed in a neutral loss scan the markers will be specifically lost from the peptide. This allows tagged peptides to be detected in a background of contamination of non-peptide and untagged peptide material by the characteristic neutral loss of the marker that is coupled to the peptide.

This has a number of advantages over the prior art methods for analysis of tagged peptides by mass spectrometry. One advantage of this method over approaches in which the marker is not cleaved from the peptide, such as ICAT, is that the ability of a peptide to undergo a characteristic neutral loss allows tagged peptides to distinguished from background material, which is not straightforward with the use of isotopic mass modifiers. This is discussed in further detail below in the section entitled “Difference Spectra”.

In addition, the use of neutral loss markers allows the charge state of the peptides to be analysed and the number of markers in the peptide to be predetermined to some extent. For example, consider the situation where a marker is coupled to cysteine side chains in a mixture of peptides. In this situation, a number of different species would be detectable in the mass spectrometer: some peptides will have only one cysteine residue, while others will have two or three or more, while some peptides will also ionise predominantly in +1, +2 or +3 charge states. Resolving these different possibilities using non-cleavable isotope tags, such as in ICAT, is very difficult. However, if a neutral loss marker which can for example undergo a neutral loss of 90 Daltons is coupled to the cysteine residues, a desired subset of peptides could be selected in the neutral loss scan. If it was desired to select only peptides with one cysteine residue, i.e. peptides coupled to only one marker, where the peptides are all in the +2 charge state, then the final quadrupole of a triple or tandem quadrupole instrument would in this example be set to scan with a mass difference of 45 Daltons relative to the first quadrupole. This scan would also detect peptides in the +4 charge state with 4 markers coupled to them, but this class of ions is much less common that peptides in the +2 state so clearly the complexity of the underlying peptide spectrum can be greatly reduced by employing a neutral loss marker and a neutral loss scan.

In embodiments of the invention, neutral loss analysis can be performed on a hybrid Time of Flight (TOF) instrument such as a Quadrupole-Time of Flight (Q-TOF) instrument or a Quadrupole Ion Trap-Time of Flight (QIT-TOF) instrument or even a Time of Flight-Time of Flight (TOF-TOF) instrument. In these instruments, ions with a pre-determined mass-to-charge ratio or range of mass-to-charge ratios can be selected in the first stages of the instrument and then subjected to Collision Induced Dissociation (CID) with subsequent detection of the fragmentation products in the final stage TOF analyser. In these hybrid TOF instruments, a sample of analytes such as peptides labelled according to this invention can be introduced into the instrument. An MS-mode spectrum can be obtained with no CID taking place to detect the unfragmented labelled peptides. Then the peptides can be subjected to CID to induce neutral loss of the neutral mass marker. Comparison of the no-CID spectrum with the CID spectrum will reveal ions that are labelled by the shift in mass that would be induced by the CID step.

For example, a 499 Dalton peptide with a single proton charge and mass marker with a mass of 50 Daltons ([M+H]⁺=550) would be detected at m/z 550 in the no-CID spectrum but in the CID spectrum a peak would appear at m/z 500. This would allow the m/z 550 peak in the no-CID spectrum to be confirmed as a labelled ion.

The use of hybrid TOF instruments is particularly advantageous as all the labelled analytes such as peptides that are introduced into the instrument at a particular time can be analysed simultaneously. Moreover, most hybrid TOF instruments support rapid switching between no-CID and CID spectra and these spectra are obtained quickly in the TOF analyser, which means that chromatographic separation of analytes can be performed and all the analytes that elute into the instrument can be detected quickly. Unlike previous methods for looking at complex peptide mixtures, the present invention can potentially identify every labelled species that is present in the sample. This is discussed in further detail below.

### Difference Spectra

The following discussion provides an exemplar method for operation of mass spectrometer to detect neutral losses from labelled analytes such as biomolecules according to this invention. As mentioned above, a key advantage of the neutral loss marker method over approaches in which the marker is not cleaved from the analyte, such as ICAT, is that the ability of a labelled analyte to undergo a characteristic neutral loss allows tagged analytes to be distinguished from background material.

In particular and as mentioned above, hybrid Time of Flight (TOF) instruments allow rapid alternation between a low or zero collision energy MS-mode analysis and a higher collision energy analysis. Note that in the context of this invention, the higher energy collision regime is preferably still a relatively low collision energy to minimise unwanted side-fragmentations. The tags of this invention are designed to fragment readily at relatively low collision energies. If desired, all of the ions entering the instrument can be subjected to the higher collision energy regime (or a collision regime under different conditions, for example varying the collision gas pulse duration to regulate collisional cooling, as discussed further below) or a range of mass-to-charge ratios can be selected for collision. The spectra obtained at low collision energy and higher effective collision energy can be compared by determining a ‘difference spectrum’, where the low energy spectrum is subtracted from the high-energy spectrum. The difference spectrum is defined as the intensity difference for each mass-to-charge ratio between the higher-energy spectrum and the low-energy spectrum. Ions whose abundance has increased in the higher-energy spectrum will have positive intensity values in the difference spectrum. Ions whose abundance has decreased in the high-energy spectrum will have negative values in the difference spectrum.

If analytes such as peptides labelled with tags of this invention are analysed in this way, then at very low or zero collision energies, the tagged analyte ions will appear in the spectrum with high relative abundance but ions corresponding to the analytes where the marker has been eliminated by neutral loss will be present at a low relative abundance. After increasing the collision energy, elimination of the neutral loss marker will be favoured so the relative intensity of the tagged analyte ions will decrease and the relative intensity of ions corresponding to the analytes where the marker has been eliminated by neutral loss will increase. This will be apparent in the difference spectrum for the high and low spectra as the tagged ions will have negative values and the analyte ions that have lost their marker will have positive intensity values.

The above concept is illustrated in FIG. 1 where simple mass modifier tags or markers according to the invention are used. In FIG. 1a, hatched box A shows intensity peaks caused by neutral loss mass marker-tagged peptide ions. Intensities of the peaks determine relative quantities. Hatched box B shows the location of the peptide ion after elimination of the neutral loss mass marker. Hatched box C demonstrates how difference spectra for the shown pattern and corresponding patterns for different charge states can be readily searched.

FIG. 1a shows that in addition to the pattern of intensity values, there will be a specific pattern of mass-to-charge ratio differences between the ions with mass markers and the ions that have lost their mass markers. This pattern will be readily identifiable using pattern matching methods known in the art. For example, cross correlation of a template pattern with the difference spectrum will allow the regions of the spectrum that have the distinct pattern of intensities and mass differences to be detected with high probability.

Difference spectra can also be determined when isobaric neutral loss mass markers are used. In this situation, ions for a given analyte (such as peptide) labelled with different isobaric neutral loss markers will all have the same mass, i.e. the tagged analyte ions for a given analyte will appear as a single peak in the MS-mode spectrum.

This concept is illustrated in FIG. 2. In FIG. 2 a hypothetical analysis of 4 peptide samples is shown. Each sample is labelled with a different isobaric neutral loss tag and then the samples are pooled for analysis. The pooled labelled peptides from each sample all have the same mass so the labelled peptides appear as a single peak in the low collision energy spectrum, as shown in the top spectrum (where hatched box A identifies the single intensity peak of peptide ions labelled with isobaric neutral loss mass markers). At increased collision energies, elimination of the neutral loss marker will be favoured so the relative intensity of the tagged peptide ion will decrease and the relative intensity of ions corresponding to the peptides where the marker has been eliminated by neutral loss will increase. Hatched box B in the middle spectrum shows the peaks of peptide ions after elimination of the isobaric neutral loss mass markers. This will be apparent in the difference spectrum for the high and low spectra as the tagged ion peak will have a negative intensity value and the peptide ions that have lost their marker will have positive intensity values. At higher collision energies, neutral loss of the mass markers results in the appearance of a peak for each sample. The intensity of these peaks will correspond to the relative quantities of the peptides in the pooled sample. Hatched box C in the lower spectrum demonstrates how difference spectra for the shown pattern and corresponding patterns for different charge states can be readily searched. Theoretical relative intensities in the lower spectrum range from +100 to −100, with the x-axis at zero.

In FIG. 3, a flowchart is shown representing the outline of the analytical process that could be used to analyse tagged analytes (referred to below as biomolecules, but applicable to other analytes) using the labels and methods of this invention. The general process is the same whether the markers are isobaric or not. The following steps may take place:
1) In the first step, tagged biomolecules are analysed in the MS-mode to collect data for the tagged species at a low collision energy. If more than one sample has been labelled the intensities of the peaks will correspond to the relative intensities of the biomolecules in their source samples;
2) Next, the tagged biomolecules are analysed in the MS-mode to collect data for the tagged species at a higher relative collision energy. A higher relative collision energy may be achieved as exemplified below by modifying collision parameters such as cooling gas pulse, without increasing the applied collision energy per se. If more than one sample has been labelled the intensities of the peaks will correspond to the relative intensities of the biomolecules in their source samples;
3) A difference spectrum is calculated by subtracting the low energy spectrum from the higher relative energy spectrum (as discussed in more detail below);
4) The characteristic shifts in mass-to-charge ratios (see for example FIGS. 1 and 2) arising from the neutral loss markers eliminating from their biomolecules are identified by fitting a template (as discussed in more detail below); and
5) The regions of the difference spectrum that match the template receive a high score while regions that do not match the template get a low or zero score. High scoring peaks in the template fitted spectrum correspond to biomolecules and these can then be subjected to further mass spectrometric analysis if desired.

### Calculation of Difference Spectra

An important step in the flow chart in FIG. 3 is the calculation of a difference spectrum. In the simplest case the intensities of ions at corresponding mass-to-charge ratios can be directly subtracted from each other. However, the overall intensities of the spectra may not be equivalent so it may be desirable to normalise the intensities of the high and low collision energy spectra prior to analysis. Normalisation could be achieved by continuously infusing one or more spike species at a predetermined concentration. The spike species are preferably chosen so that they do not fragment significantly at the collision energies used to dissociate neutral loss markers. The resulting high and low energy spectra containing spike ions can then be normalised by scaling them using the intensities of the spike species. If only one spike is used then the high and low energy spectra are normalised by dividing all the intensities in the spectrum by the intensity of the spike ion from that spectrum. The spectra can then be scaled again by multiplying both by the same factors if desirable to get suitable units. If more than one spike is used with different mass-to-charge ratios then the user will have to decide how to scale using those spikes. For example, if two spikes are used at m/z 500 and m/z 1500, then the lower mass spike could be used to scale the range from 0 to 1000 while the higher spike could scale the range of 1001 to 2000 if desired. If multiple spikes are used a spline curve can be fitted to these peaks and a continuous scaling function for every mass can be determined.

Another approach to normalise data is to use quantile normalisation. Quantile normalisation assumes that the total ion count for the high and low spectra should be the same but the distribution of intensities will be different. The spectra are thus normalised by sorting all the peaks in the spectrum in order of ion intensity. The intensity of each rank is then set to the mean of the intensity for that rank in both spectra, i.e. if the 10^{th} most intense species in the low energy spectrum has an intensity of 20 and the 10^{th} most intense species in the high energy spectrum has an intensity of 30, then the 10^{th} most intense species in both spectra will become 25 after quantile normalisation. The data is then re-ordered according to mass-to-charge ratio to restore the normalised spectra. Since the total number of peaks in the high and low energy spectra might differ, some of the lower intensity peaks at the high end of the spectra could be discarded until the two spectra have the same number of peaks. Similarly, peaks at the low mass end of the spectrum can be discarded if desired as very low mass species are likely to be noise.

After normalisation the low energy spectrum can be subtracted from the corresponding high energy spectrum to produce a difference spectrum.

### Fitting Templates to the Difference Spectrum

A template corresponding to the expected mass differences that will be generated from a particular labelling scheme can be determined as exemplified in FIG. 1b. If 4 non-isobaric neutral loss markers are used, each differing by 6 Daltons from each other and the lowest mass marker having a mass of 50 Daltons, then the template (labelled “T”) would expect to find the unlabelled ion at a mass-to-charge ratio that is 50 Daltons below the ion labelled with the marker with the lowest mass, or rather the first labelled species will be found at 50 Daltons above the unlabelled ion. Similarly there will be labelled ion peaks at 56, 62 and 68 Daltons above the unlabelled species. Typically the template would not be fitted to the very low mass end of the spectrum. Template fitting might start at 400 Daltons, in a practical situation. Thus, the first template would expect an unlabelled species at m/z 400 Daltons in the difference spectrum. If the low energy spectrum has been subtracted from the high energy spectrum then, the unlabelled species would be expected to have a positive value in the difference spectrum as the intensity of the unlabelled species would be expected to increase upon CID. The labelled species would be expected to be present at m/z 450, 456, 462 and 468. If the low energy spectrum has been subtracted from the high energy spectrum then the labelled species would be expected to have negative values in the difference spectrum as their intensity will decrease upon CID. The relative magnitudes of the labelled and unlabelled species will also probably fall within an expected range of ratios. Thus the template would comprise an ion at 450 with a positive magnitude and a series of 4 ions with a negative magnitude corresponding to labelled and unlabelled species respectively. The ratio of those magnitudes will fall within an expected range, which would have to be determined empirically by analysis of a number of labelled molecules. This ratio will depend on various factors including the tag design, the collision energy and the size of the labelled peptide or other biomolecule.

The intensities of the tagged species may vary from sample to sample, however this information will not be known in advance so the template will typically have fixed intensities for the labelled species. To allow for this when fitting the template, the peaks in the difference spectrum that might correspond to the labelled species could be temporarily adjusted so that they all adopt the mean of the intensities of the putative labelled species, i.e. the peaks at 450, 456, 462 and 468 in the first template fitting might have intensities of −10, −20, −30 and −40 respectively in the difference spectrum, but these would all be adjusted to the mean (−25).

Finally, the template may have to be scaled to be approximately the same as the region of the difference spectrum being compared. This can be done by scaling the first peak in the template so that it has the same intensity as the first peak to be compared in the difference spectrum.

The similarity between the template and the region of the difference spectrum under analysis can then be determined. Scoring the fit of the template to the spectrum can be performed using various methods. Typically, this is done by cross correlation (see Smith, 1997, “The Scientist and Engineer's Guide to Digital Signal Processing”, California Technical Publishing).

After scoring the first location in the difference spectrum against the template, the template would be moved along by 1 Dalton, i.e. if it started at m/z 400 then the next template would be fitted at 401 next and so on. A new spectrum is then determined that is the score for fitting the template at each point in the spectrum. Regions of this “template score” spectrum with high scores correspond to high probabilities that the underlying peaks correspond to labelled molecules of interest.

If 4 isobaric neutral loss markers are used (see FIG. 2) having a total mass of 60 Daltons but neutral loss fragments each differing by 6 Daltons from each other and the lowest neutral loss fragment having a mass of 20 Daltons, then the template would expect to find all the labelled ions at a mass-to-charge ratio that is 40 Daltons above the ion labelled with the neutral loss fragment with the lowest mass. Similarly there will be ion peaks at 34, 28 and 22 Daltons below the labelled species corresponding to the different possible neutral losses. Typically the template would not be fitted to the very low mass end of the spectrum. Template fitting might start at 400 Daltons, in a practical situation. Thus, the first template would expect a labelled species at m/z 440 Daltons in the difference spectrum. If the first (for example low) energy spectrum has been subtracted from the second (for example, high) energy spectrum then, the labelled species would be expected to have a negative value in the difference spectrum as the intensity of the labelled species would be expected to decrease upon CID. The neutral loss fragment species would be expected to be present at m/z 400, 406, 412 and 418. If the first (for example, low) energy spectrum has been subtracted from the second (for example, high) energy spectrum then the neutral loss species would be expected to have positive values in the difference spectrum as their intensity will increase upon CID. The relative magnitudes of the labelled and unlabelled species will also probably fall within an expected range of ratios. Thus the template would comprise an ion at 440 with a negative magnitude and a series of 4 ions with a positive magnitude corresponding to labelled and neutral loss species respectively. The ratio of those magnitudes will fall within an expected range, which would have to be determined empirically by analysis of a number of labelled molecules. This ratio will depend on various factors including the tag design, the collision energy and the size of the labelled peptide or other biomolecule.

The intensities of the neutral loss species may vary from sample to sample, however this information will not be known in advance so the template will typically have fixed intensities for the labelled species. To allow for this when fitting the template, the peaks in the difference spectrum that might correspond to the labelled species could be temporarily adjusted so that they all adopt the mean of the intensities of the putative labelled species, i.e. the peaks at m/z 400, 406, 412 and 418 in the first template fitting might have intensities of +10, +20, +30 and +40 respectively in the difference spectrum, but these would all be adjusted to the mean (+25).

Finally, the template may have to be scaled to be approximately the same as the region of the difference spectrum being compared. This can be done by scaling the first peak in the template so that it has the same intensity as the first peak to be compared in the difference spectrum.

The similarity between the template and the region of the difference spectrum under analysis can then be determined. Scoring the fit of the template to the spectrum can be performed using various methods. Typically, this is done by cross correlation (see Smith, 1997, above).

After scoring the first location in the difference spectrum against the template, the template would be moved along by 1 Dalton, i.e. if it started at m/z 400 then the template would be fitted at 401 next and so on. A new spectrum is then determined that is the score for fitting the template at each point in the spectrum. Regions of this “template score” spectrum with high scores correspond to high probabilities that the underlying peaks correspond to labelled molecules of interest.

### Relative Abundance Determination by Neutral Loss Analysis

In many analyses it is desirable to determine the relative abundance in different samples of complex mixtures of analytes such as for example biomolecules. Of particular interest are mixtures of peptides, such as peptide digests of proteins from diseased or normal tissues. Typically, quantification of analytes such as peptides is carried out by labelling one sample with one isotope of a marker and a second with a different isotope marker. In this way, there will be two peaks in the mass spectrum for corresponding analytes in each sample. The relative intensity of these peaks will give an accurate measurement of the relative abundance of the analytes in their respective samples. Quantification according to the methods of this invention can be achieved with sets of neutral loss markers, which are isotopes of each other.

In the certain aspects of the invention, a set of two or more neutral loss markers is provided. FIG. 9 illustrates a pair of example markers and their use. This marker is comprised of a central dipeptide of proline and aspartic acid. This dipeptide undergoes a facile neutral scission upon low energy collision. In FIG. 9 the introduction of a reactive group is achieved by coupling an iodacetyl moiety to the aspartic acid moiety via its amino group. The iodoacetyl functional group allows the marker to be easily coupled to a thiol group, e.g. the cysteinyl thiol groups that are often present in peptides. This coupling is shown in FIG. 9 where in step (1) the two marker variants are coupled to two peptides from different samples via cysteinyl thiol groups. The subsequent cleavage that would take place after a low energy collision is shown in step (2). It can be seen that the proline function will be lost from the peptide as a neutral species. As long as the peptides that are labelled are able to ionise in their own right, the peptide will be detectable and the presence of the markers can be inferred from analysis of the neutral loss induced by low energy collision.

The markers shown in FIG. 9 can be used to quantify peptides in a pair of samples if each marker is used to label peptides in a different sample. Since the markers are isotopes of each other the labelled forms of corresponding peptides from each sample will also be isotopes of each other and will behave in an equivalent manner in chromatographic separations. This means that corresponding peptides will be ionised in the mass spectrometer simultaneously as they will elute from standard single or multi-dimensional chromatographic or electrophoretic separations at the same time. Thus pairs of corresponding labelled peptides will appear in a neutral loss mass spectrum as a pair of ion peaks. The ratio of the intensities of the ion peaks will correspond to the ratios of the quantities of the peptides in their parent samples.

It should be apparent that it is possible to generate more than two neutral loss markers with the same chemical structure such as those in FIG. 9 by employing additional isotope variants of the components of the markers to generate more marker molecule isotopes with different masses.

In a further embodiment of the invention, pairs of markers of the form shown in FIG. 8 are used. In this embodiment, the pair of markers has the same overall mass but the neutral loss fragments have different masses, which are normalised by the presence of mass normaliser functions with appropriate masses. Thus one marker comprises a proline residue with four ¹³C atoms and the mass normaliser, aspartic acid, comprises normal ¹²C and ¹⁴N atoms. In contrast, the second marker comprises proline with four normal ¹²C atoms and the mass normaliser, aspartic acid, comprises three ¹³C atoms and one ¹⁵N atom. In this way, two isobaric markers are generated, which have a differing internal mass distribution. If peptides in two different samples are labelled with markers of this kind, corresponding tagged peptides from each sample will have the same mass and will ionise to give ions with the same mass-to-charge ratio. In the first stage of a neutral loss analysis these ions will be selected together. After low energy collision, cleavage of the neutral loss fragment will produce a pair of ions separated by the difference in mass between the neutral loss fragments. This type of marker is particularly advantageous for neutral loss analysis on Quadrupole-Time of Flight instrument or for neutral loss analysis in Ion Trap instruments. This sort of marker can be exploited in a number of ways, as described herein.

Shotgun analysis techniques such as MudPIT (Washburn et al., 2001, Nat. Biotechnol. 19(3): 242-247) involve generation of a peptide digest from a mixture of proteins, separation of the peptides by chromatography (preferably more than one different stage of chromatography) and injection of the peptides into a mass spectrometer, where automated selection and fragmentation of peptide ions takes place to allow peptides to be sequenced. An embodiment of the invention using markers of the type shown in FIG. 8 can provide an improved method of shotgun analysis. This improved method allows both the sequence and the abundance of corresponding peptides in two or more samples comprising a complex peptide mixture to be determined. This improved method also simplifies the analysis of the fragmentation patterns to make sequencing easier.

In an example of this embodiment of the invention, the pair of markers shown in FIG. 8 would be used. Each marker would be used to label peptides in one sample of a pair of protein digests. In this embodiment, the peptides are preferably labelled at one terminus, either at N-terminus or C-terminus. Methods for achieving this are discussed in more detail below.

As mentioned above, the mass normalised markers would ensure that the labelled peptides would have the same overall masses and would also behave in a similar fashion during chromatographic or electrophoretic separations. Thus, in the subsequent shotgun analysis of the tagged peptide samples, each corresponding peptide pair would be selected simultaneously. If the labelled peptides are fragmented to generate a sequence spectrum, loss of the neutral fragments will result in a sequence spectrum comprised of ion doublets, where the ratio of the intensities of the paired peaks will be indicative of the ratios of the peptides in the sample. In addition, since ions corresponding to real sequence fragments will be paired and the ion pairs should have a consistent ratio, it will be possible to filter background signals that do not have these characteristics out of the spectrum allowing identification of sequence fragments more easily. Alternatively, if a Trap-TOF geometry is used, an MS³ method can be used. The isobarically labelled peptides can be isolated in the trap then the neutral loss can be induced. The remaining peptide ions can be retained in the trap. The peptide ion with the highest intensity can then be selected for sequencing.

### Synthesis of Neutral Loss Mass Markers

Certain neutral loss mass markers of this invention may be readily produced using a peptide synthesiser. Exemplar marker compounds of the invention are modified peptides. Peptide synthesis provides chemical diversity allowing for a wide range of markers with chosen properties to be produced in an automated fashion.

### Automated Peptide Synthesis for Marker Preparation

The mass markers of this invention can be prepared using a number of peptide synthesis methods that are well known in the art (see for example Jones, 1991, “The chemical synthesis of peptides”, Oxford University Press; Fields & Noble, 1990, Int J Pept Protein Res 35(3): 161-214; Albericio, 2000, Biopolymers 55(2): 123-139). In addition, the use of peptide and peptide-like markers enables coupling of these markers to peptides using conjugation techniques well known in the art.

Modern peptide synthesis is typically carried out on solid phase supports in automated synthesiser instruments, which deliver all the necessary reagents for each step of a peptide synthesis to the solid support and remove spent reagents and unreacted excess reagents at the end of each step in the cycle. Solid phase peptide synthesis is, however, often performed manually, particularly when specialist reagents are being tested for the first time. In essence peptide synthesis involves the addition of N-protected amino acids to the solid support. The peptide is normally synthesised with the C-terminal carboxyl group of the peptide attached to the support, and the sequence of the peptide is built from the C-terminal amino acid to the N-terminal amino acid. The C-terminal amino acid is coupled to the support by a cleavable linkage. The N-protected alpha amino group of each amino acid is deprotected to allow coupling of the carboxyl group of the next amino acid to the growing peptide on the solid support. For most purposes, peptide synthesis is performed by one of two different synthetic procedures, which are distinguished by the conditions needed to remove the N-protecting group. The tert-butyloxycarbonyl (t-BOC) group is cleaved by mildly acidic conditions, e.g. trifluoroacetic acid in dichloromethane, while the fluorenylmethoxycarbonyl (FMOC) group is cleaved by mildly basic conditions, e.g. 20% piperidine in dimethylformamide. Reactive side chains in amino acids also need protection during cycles of amide bond formation. These side chains include the epsilon amino group of lysine, the guanidino side-chain of arginine, the thiol functionality of cysteine, the hydroxyl functionalities of serine, threonine and tyrosine, the indole ring of tryptophan and the imidazole ring of histidine. The choice of protective groups used for side-chain protection is determined by the cleavage conditions of the alpha-amino protection groups, as the side-chain protection groups must be resistant to the deprotection conditions used to remove the alpha-amino protection groups. A first protective group is said to be ‘orthogonal’ to a second protective group if the first protective group is resistant to deprotection under the conditions used for the deprotection of the second protective group and if the deprotection conditions of the first protecting group do not cause deprotection of the second protecting group.

Examples of side-chain protection groups compatible with FMOC syntheses are shown in Table 1.

TABLE 1

| Side Chain | Protective Group |
|---|---|
| Epsilon amino group of lysine | t-BOC group |
| Guanidino-functionality | Nitro group or 2,2,5,7,8- |
| of arginine | pentamethylchroman-6-sulphonyl group |
| Imidazole ring of histidine | τ-Trityl group, π-benzyloxymethyl (Bom) |
| | group. |
| Hydroxyl functionalities of | Tert-butyl group |
| serine, threonine and tyrosine | |
| Indole ring of tryptophan | t-BOC |
| Thiol functionality of cysteine | trityl or benzyl group |
| Amide functionalities of | Not usually necessary but Trityl group can |
| glutamine and asparagine | be used for example. |
| Carboxylic acid functionalities | Tert-butyl group |
| of glutamic acid and | |
| aspartic acid. | |
| Thioether of methionine | Sometimes protected as sulphoxide |

Other side-chain protective groups that are orthogonal to FMOC protection will be known to one of ordinary skill in the art and may be applied with this invention (see for example Fields & Noble, 1990, above).

Protection groups for reactive side-chain functionalities compatible with t-BOC synthesis are shown below in Table 2.

TABLE 2

| Side Chain | Protective Group |
|---|---|
| Epsilon amino group of lysine | Benzyloxycarbonyl (Z) group |
| Guanidino-functionality | Not usually necessary but nitration can be |
| of arginine | used |
| Imidazole ring of histidine | π-benzyloxymethyl (Bom) group. |
| Hydroxyl functionalities of | Benzyl group |
| serine, threonine and tyrosine | |
| Hydroxyl functionality | 2-Bromobenzyloxycarbonyl group |
| of tyrosine | |
| Indole ring of tryptophan | Formyl |
| Thiol functionality of cysteine | Benzyl group |
| Amide functionalities of | Not usually necessary? |
| glutamine and asparagine | |
| Carboxylic acid functionalities | Benzyl ester group |
| of glutamic acid and | |
| aspartic acid. | |
| Thioether of methionine | Sometimes protected as sulphoxide |

Again, the practitioner of ordinary skill in the art will be aware of other protective groups for use with reactive side chains that are orthogonal to t-BOC alpha amino protection. Various different solid supports and resins are commercially available for peptide synthesis using either the FMOC or t-BOC procedures (for a review of solid supports see Meldal, 1997, Methods Enzymol 289: 83-104).

### Mass Modified Amino Acids

An advantage of using conventional automated peptide synthesis for the preparation of the markers of this invention arises from the availability of a number of commercially available isotopically mass modified amino acids. Some of these are shown in Table 3.

TABLE 3

| Amino acid | Isotope Forms |
|---|---|
| Alanine | CH₃CH(NH₂)¹³CO₂H, CH₃CD(NH₂)CO₂H, CH₃¹³CH(¹⁵NH₂)CO₂H, |
| | CD₃CH(NH₂)CO₂H, CD₃CD(NH₂)CO₂H, CD₃CH(NH₂)¹³CO₂H, |
| | CD₃¹³CH(NH₂)CO₂H, ¹³CH₃¹³CH(¹⁵NH₂)¹³CO₂H, |
| Arginine | [(¹⁵NH₂)₂CNHCH₂CH₂CH(NH₂)CO₂H]⁺ |
| Asparagine | H₂N¹³COCH₂CH(NH₂)CO₂H, H₂N¹³CO¹³CH₂¹³CH(NH₂)¹³CO₂H, |
| | H₂¹⁵NCOCH₂CH(NH₂)CO₂H, H₂¹⁵NCOCH₂CH(¹⁵NH₂)CO₂H, |
| Aspartic Acid | HO₂¹³CCH₂CH(NH₂)CO₂H, HO₂C¹³CH₂CH(NH₂)CO₂H, |
| | HO₂CCH₂CH(NH₂)¹³CO₂H, HO₂¹³CCH₂CH(NH₂)¹³CO₂H, |
| | HO₂CCH₂¹³CH(NH₂)¹⁵CO₂H, HO₂¹³C¹³CH₂CH(NH₂)CO₂H, |
| | HO₂¹³C¹³CH₂¹³CH(NH₂)¹³CO₂H, HO₂CCD₂C(NH₂)CO₂H, |
| | HO₂CCH₂CH(¹⁵NH₂)CO₂H, HO₂CCH₂CH(¹⁵NH₂)¹³CO₂H, |
| Glutamic Acid | HO₂CCH₂CH₂CH(NH₂)¹³CO₂H, HO₂CCH₂CH₂¹³CH(NH₂)CO₂H, |
| | HO₂CCH₂¹³CH₂CH(NH₂)CO₂H, HO₂C¹³CH₂CH₂CH(NH₂)CO₂H, |
| | HO₂¹³CCH₂CH₂CH(NH₂)CO₂H, |
| | HO₂¹³C¹³CH₂¹³CH₂¹³CH(NH₂)¹³CO₂H, |
| | HO₂CCD₂CH₂CH(NH₂)CO₂H, HO₂CCD₂CD₂CD(NH₂)CO₂H, |
| | HO₂¹³C¹³CH₂¹³CH₂¹³CH(¹⁵NH₂)¹³CO₂H |
| Glutamine | H₂NCOCH₂CH₂CH(NH₂)¹³CO₂H, H₂N¹³COCH₂CH₂CH(NH₂)CO₂H, |
| | H₂NCOCD₂CD₂CD(NH₂)CO₂H, H₂¹⁵NCOCH₂CH₂CH(NH₂)CO₂H, |
| | H₂NCOCH₂CH₂CH(¹⁵NH₂)CO₂H, |
| | H₂¹⁵NCOCH₂CH₂CH(¹⁵NH₂)CO₂H, |
| | H₂¹⁵NCOCH₂CH₂CH(¹⁵NH₂)CO₂H, |
| | H₂¹⁵N¹³CO¹³CH₂¹³CH₂¹³CH(¹⁵NH₂)¹³CO₂H, |
| Glycine | H₂NCH₂¹³CO₂H, H₂N¹³CH₂CO₂H, H₂N¹³CH₂¹³CO₂H, |
| | H₂NCD₂CO₂H, H₂¹⁵NCH₂CO₂H, H₂¹⁵N¹³CH₂CO₂H, |
| | H₂¹⁵NCH₂¹³CO₂H, H₂¹⁵N¹³CH₂¹³CO₂H, |
| Histidine | (CH₂)₂N₂CCH₂CH(NH₂)¹³CO₂H, (CH)₂N₂CCH₂CH(¹⁵NH₂)CO₂H, |
| | (CH)₂¹⁵N₂CCH₂CH(NH₂)CO₂H |
| Leucine | (CH₃)₂CHCH₂CH(NH₂)¹³CO₂H, (CH₃)₂CHCH₂¹³CH(NH₂)CO₂H, |
| | (CH₃)₂CHCH₂¹³CH(NH₂)¹³CO₂H, (CH₃)₂CHCH₂CD(NH₂)CO₂H, |
| | (CH₃)₂CHCD₂CD(NH₂)CO₂H, (CD₃)(CH₃)CHCH₂CH(NH₂) CO₂H, |
| | (CD₃)₂CDCH₂CH(NH₂)CO₂H, (CD₃)₂CDCD₂CD(NH₂)CO₂H, |
| | (CH₃)₂CHCH₂CH(¹⁵NH₂)CO₂H, (CH₃)₂CHCH₂CH(¹⁵NH₂)¹³CO₂H |
| Lysine | H₂NCH₂CH₂CH₂CH₂CH(NH₂)¹³CO₂H, |
| | H₂NCH₂CH₂CH₂CH₂¹³CH(NH₂)CO₂H, |
| | H₂N¹³CH₂CH₂CH₂CH₂CH(NH₂)CO₂H, |
| | H₂NCH₂CH₂CH₂CH₂¹³CH(NH₂)¹³CO₂H, |
| | H₂NCD₂CD₂CD₂CH₂CH(NH₂)CO₂H, |
| | H₂NCD₂CD₂CD₂CD₂CH(NH₂)CO₂H, |
| | H₂NCH₂CH₂CH₂CH₂CH(¹⁵NH₂)CO₂H, |
| | H₂¹⁵NCH₂CH₂CH₂CH₂CH(NH₂)CO₂H, |
| | H₂¹⁵N¹³CH₂CH₂CH₂CH₂CH(NH₂)CO₂H, |
| Methionine | CH₃SCH₂CH₂CH(NH₂)¹³CO₂H, CH₃SCH₂CH₂¹³CH(NH₂)CO₂H, |
| | ¹³CH₃SCH₂CH₂CH(NH₂)CO₂H, CH₃SCH₂CH₂CD(NH₂)CO₂H, |
| | CD₃SCH₂CH₂CH(NH₂)CO₂H, CH₃SCH₂CH₂CH(¹⁵NH₂)CO₂H, |
| | ¹³CD₃SCH₂CH₂CH(NH₂)CO₂H, CH₃SCH₂CH₂¹³CH(¹⁵NH₂)CO₂H, |
| Phenylalanine | C₆H₅CH₂CH(NH₂)¹³CO₂H, C₆H₅CH₂¹³CH(NH₂)CO₂H, |
| | ¹³C₆H₅CH₂CH(NH₂)CO₂H, C₆H₅CH₂CD(NH₂)CO₂H, |
| | C₆H₅CD₂CH(NH₂)CO₂H, C₆D₅CH₂CH(NH₂)CO₂H, |
| | C₆D₅CD₂CD(NH₂)CO₂H, C₆H₅CH₂CH(¹⁵NH₂)CO₂H |
| Proline | 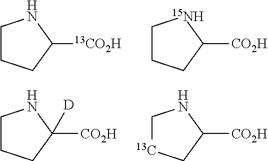 |
| Serine | HOCH₂CH(NH₂)¹³CO₂H, HOCH₂¹³CH(NH₂)CO₂H, |
| | HO¹³CH₂CH(NH₂)CO₂H, HOCH₂CH(¹⁵NH₂)CO₂H, |
| | HOCH₂¹³CH(¹⁵NH₂)CO₂H, |
| Threonine | CH₃CH(OH)CH(NH₂)¹³CO₂H |
| Tryptophen | 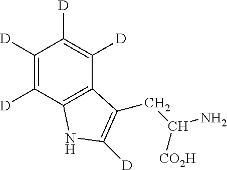 |
| Tyrosine | HO(C₆H₄)CH₂CH(NH₂)¹³CO₂H, HO(C₆H₄)CH₂¹³CH(NH₂)CO₂H, |
| | HO(C₆H₄)¹³CH₂CH(NH₂)CO₂H, |
| | HO(C₆H₄)¹³CH₂¹³CH(NH₂)¹³CO₂H, |
| | HO(¹³C₆H₄)CH₂CH(NH₂)CO₂H, |
| | HO(¹³C₆H₄)¹³CH₂¹³CH(NH₂)¹³CO₂H, |
| | HO(C₆H₄)CD₂CH(NH₂)CO₂H, |
| | HO(C₆D₂H₂)CH₂CH(NH₂)CO₂H, HO(C₆D₄)CH₂CH(NH₂)CO₂H, |
| | HO(C₆H₄)CH₂CH(¹⁵NH₂)CO₂H, H¹⁷O(C₆H₄)CH₂CH(NH₂)CO₂H, |
| | H¹⁸O(C₆H₄)CH₂CH(NH₂)CO₂H, |
| | HO(C₆H₄)CH₂¹³CH(¹⁵NH₂)CO₂H, |
| | HO(¹³C₆H₄)¹³CH₂¹³CH(¹⁵NH₂)¹³CO₂H |
| Valine | (CH₃)₂CHCH(NH₂)¹³CO₂H, (CH₃)₂CH¹³CH(NH₂)CO₂H, |
| | (CH₃)₂CHCD(NH₂)CO₂H, (CD₃)₂CDCD(NH₂)CO₂H, |
| | (CH₃)₂CHCH(¹⁵NH₂)CO₂H, |

For many of the above amino acids, both the D- and L-forms are available (from ISOTEC Inc., Miamisburg, Ohio, US, for example), either of which may be used in the preparation of the markers of this invention. Mixtures of D and L forms are also available but are less preferred if the markers of this invention are to be used in chromatographic separations. For some, FMOC or t-BOC protected derivatives are also available. Mass modified amino acids based on substitution of deuterium for hydrogen and on substitution of ¹³C and ¹⁵N isotopes for ¹²C and ¹³N isotopes are also available and are equally applicable for the synthesis of the markers of this invention. Various amino acids that are not typically found in peptides may also be used in the markers of this invention, for example deuterated forms of amino-butyric acid are commercially available. For the purposes of this invention non-radioactive, stable isotopes can be used for safety reasons.

Fluorinated derivatives of a number of amino acids are also available. Some of the commercially available fluorinated amino acids are shown in Table 4.

TABLE 4

| Amino acid | Fluorinated Forms |
|---|---|
| Glutamic Acid | HO₂CCFHCH₂CH(NH₂)CO₂H, |
| Leucine | (CH₃)(CF₃)CHCH₂CH(NH₂)CO₂H |
| Phenylalanine | C₆FH₄CH₂CH(NH₂)CO₂H, C₆F₂H₃CH₂CH(NH₂)CO₂H, |
| | C₆F₃H₂CH₂CH(NH₂)CO₂H, |
| Phenylglycine | C₆FH₄CH(NH₂)CO₂H, C₆F₂H₃CH(NH₂)CO₂H, |
| | C₆F₃H₂CH(NH₂)CO₂H, |
| Valine | (CH₃)₂CFCH(NH₂)CO₂H |

For most of the above fluorinated amino acids, the reagents are available as mixtures of D and L forms. In general, fluorinated variants of amino acids are less preferred than isotope substituted variants. The fluorinated compounds can be used to generate a range of mass markers with the same mass but each marker will be chemically different, which means that their behaviour in the mass spectrometer will vary more than isotope substituted markers. Moreover, the markers will not have identical chromatographic properties if the markers are to be used in chromatographic separations.

### Reactive Functionalities

In aspects of the invention, the mass markers comprise a reactive functionality. In the simplest embodiments this may be an N-hydroxysuccinimide ester introduced by activation of the C-terminus of the marker peptides of this invention. In conventional peptide synthesis, this activation step would have to take place after the peptide mass marker has been cleaved from the solid support used for its synthesis. An N-hydroxysuccinimide activated peptide mass marker could also be reacted with hydrazine to give a hydrazide reactive functionality, which can be used to label periodate oxidised sugar moieties, for example. Amino-groups or thiols can be used as reactive functionalities in some applications and these may be introduced by adding lysine or cysteine after amino acid 2 of the marker peptide. Lysine can be used to couple markers to free carboxyl functionalities using a carbodiimide as a coupling reagent. Lysine can also be used as the starting point for the introduction of other reactive functionalities into the marker peptides of this invention. The thiol-reactive maleimide functionality can be introduced by reaction of the lysine epsilon amino group with maleic anhydride. The cysteine thiol group can be used as the starting point for the synthesis of a variety of alkenyl sulphone compounds, which are useful protein labelling reagents that react with thiols and amines. Compounds such as aminohexanoic acid can be used to provide a spacer between the mass modified amino acids and the reactive functionality.

### Affinity Capture Ligands

In certain embodiments of the invention, the mass markers comprise an affinity capture ligand. Affinity capture ligands are ligands which have highly specific binding partners. These binding partners allow molecules tagged with the ligand to be selectively captured by the binding partner. A solid support may be derivatised with the binding partner so that affinity ligand tagged molecules can be selectively captured onto the solid phase support. A useful affinity capture ligand is biotin, which can be readily introduced into (peptide) mass markers of this invention by standard methods known in the art. In particular a lysine residue may be incorporated after amino acid 2 through which an amine-reactive biotin can be linked to the peptide mass markers (see for example Geahlen et al., 1992, Anal Biochem 202(1): 68-67; Sawutz et al., 1991, Peptides 12(5): 1019-1012; Natarajan et al., 1992, Int J Pept Protein Res 40(6): 567-567). Iminobiotin is also applicable. A variety of avidin counter-ligands for biotin are available, which include monomeric and tetrameric avidin and streptavidin, all of which are commercially available on a number of solid supports, including magnetic beads.

Other affinity capture ligands include digoxigenin, fluorescein, nitrophenyl moieties and a number of peptide epitopes, such as the c-myc epitope, for which selective monoclonal antibodies exist as counter-ligands. Metal ion binding ligands such as hexahistidine, which readily binds Ni²⁺ ions, are also applicable. Chromatographic resins, which present iminodiacetic acid chelated Ni²⁺ ions are commercially available, for example. These immobilised nickel columns may be used to capture peptide mass markers, which comprise oligomeric histidine. As a further alternative, an affinity capture functionality may be selectively reactive with an appropriately derivatised solid phase support. Boronic acid, for example, is known to selectively react with vicinal cis-diols and chemically similar ligands, such as salicylhydroxamic acid. Reagents comprising boronic acid have been developed for protein capture onto solid supports derivatised with salicylhydroxamic acid (Stolowitz et al., 2001, Bioconjug Chem 12(2): 229-239; Wiley et al., 2001, Bioconjug Chem 12(2): 240-250). It is expected that a phenylboronic acid functionality could be linked to a peptide mass marker according to the invention to generate capture reagents that can be captured by selective chemical reactions. The use of this sort of chemistry would not be directly compatible with biomolecules bearing vicinal cis-diol-containing sugars. However, these sorts of sugars could be blocked with phenylboronic acid or related reagents prior to reaction with boronic acid derivatised peptide mass marker reagents.

Amino Labelling with Dicarboxylic Anhydrides

It is known that dicarboxylic anhydrides such as succinic anhydride, maleic anhydride, citraconic anhydride, dimethyl maleic anhydride and phthalic anhydrides react reversibly with primary amines in proteins to form amides (Palacián et al., 1990, Mol Cell Biochem. 97(2): 101-11; de la Escalera & Palacián, 1989, Biochem Cell Biol. 67(1): 63-6; Riley & Perham, 1970, Biochem J. 118(5):733-9). Conventionally, these reactions have been reversed by heating solutions of proteins labelled with these reagents in the presence of acid. However, we have found that the amide derivatives produced by these reagents will also cleave when subjected to collision induced dissociation (CID) in a mass spectrometer. Ease of cleavage by CID is similar to the pattern for acid and heat induced cleavage, the more structurally constrained anhydrides cleave more easily than the less structurally constrained compounds, i.e. succinic acid cleaves the least well while the dimethyl maleic anhydride cleaves very well.

Isotope substituted forms of some of these compounds, such as maleic anhydride, citraconic anhydride, succinic anhydride and phthalic anhydride, are also commercially available (Cambridge Isotope Laboratories, Inc; Andover, Mass., USA and Sigma Aldrich, UK) and suitable for use with the methods of this invention.

FIG. 4 illustrates 2 samples of the same peptide having an amino acid sequence GLGEHNIDVLEGNEQFINAAK [SEQ ID NO: 4], labelled with O-methylisourea on the epsilon amino groups and with different isotopes of maleic anhydride on the alpha amino groups. FIG. 4 also illustrates collision induced dissociation of the labelled peptides to recover the peptide without the anhydride label at the alpha amino groups.

It is expected that derivatives of these compounds will also be useful as mass markers for the practice of this invention. For the purposes of this invention, dicarboxylic anhydrides may be regarded as both collision cleavable groups and preferred reactive functions for use with the more complex tags of this invention. The use of this class of compound is not restricted to the analysis of peptides. For example, amino-derivatised oligonucleotides could be readily labelled with dicarboxylic acid anhydride compounds.

### Amino Labelling by Reductive Alkylation

In some embodiments, coupling of the tags of the invention to their target molecule or analyte should not change the overall charge, or only minimally change the charge, of the molecule or analyte. Reductive alkylation of amino groups is a suitable method for achieving this. Aldehydes or ketones will react reversibly with amino groups to form an imine or Schiff's base. The imine can be reduced to an amine by addition of appropriate reducing agents such as sodium borohydride or sodium cyanoborohydride. The reduced amino groups are stable for subsequent analysis by mass spectrometry.

Tags for the purpose of this invention can be prepared by peptide synthesis as discussed above. Aldehyde functions can be introduced into peptides by various means. C-terminal aldehydes can be introduced by using pre-loaded resins that release the peptide as an aldehyde in the final cleavage step (Ede et al., 2000, J Pept Sci. 6(1): 11-18). Resins for this purpose are commercially available from Novabiochem (a subsidiary of Merck Biosciences—Merck KGaA, Darmstadt, Germany).

Aldehydes may be introduced into peptides in a variety of other ways such as oxidation of a peptide alcohol (Woo et al., 1995, Bioorg. Med. Chem. Lett. 5 (14): 1501-1504), oxidation of a diol (Zhang et al., 1998, Proc Natl Acad Sci USA. 95(16): 9184-9189), reduction of a peptide Weinreb amide (Guichard et al., 1993, Pept Res. 6(3): 121-124; Fehrentz et al., 1995, Tetrahedron Letters 36(43): 7871-7874). An example of the synthesis of a small peptide tag with a C-terminal aldehyde is shown in FIG. 5.

As for other classes of label, the use of reductive alkylation is not restricted to the analysis of peptides. Amino-labelled oligonucleotides would also be suitable substrates for reductive alkylation.

Example reagents are shown in FIG. 5. FIG. 5a illustrates a tag based on a short peptide, (N)-acetyl-aspartic acid-proline-aldehyde linker-(C). FIG. 5b illustrates a tag based on a short peptide, (N)-acetyl-alanine-proline-aldehyde-(C). The aspartic acid in FIG. 5a and the alanine residue in FIG. 5b comprise a number of ¹³C and ¹⁵N isotopes. ‘Light’ versions of the tag can also be synthesised without these isotopes. The acetyl group can optionally comprise isotopes and other carboxylic acids can be used to block the amines of the aspartic acid and alanine residues in FIGS. 5a and 5b respectively. These carboxylic acids can be used to diversify the range of masses and isotopes in these tags. The labelling by reductive alkylation (“RA”) is shown giving an amino group. Aldehydes often react twice at the primary amines although this is not shown for simplicity. The CID cleavage mechanisms for the aspartic acid peptide and the alanine peptides are also shown.

### Amine Labelling by Guanidination

Guanidination with O-methylisourea is a well known derivatisation procedure for specific modification of epsilon amino groups of peptides and proteins (Ji & Guo, 2005, J Proteome Res. 4(6): 2099-2108; Zappacosta & Annan, 2004, Anal Chem. 76(22): 6618-6627; Brancia et al., 2004, Anal Chem. 76(10): 2748-2755; Beardsley & Reilly, 2002, Anal Chem. 74(8): 1884-1890). The guanidino group retains the positive charge on the epsilon amino group of peptides. As such, mass modifiers that react with amino groups by guanidination are suitable for use with this invention.

More complex reagents that react in a similar manner to O-methylisourea are also known such as 2-methoxy-4,5-dihydro-1H-imidazole (Peters et al., 2001, Rapid Commun Mass Spectrom. 15(24): 2387-2392). It is expected that this imidazole function could be readily introduced as a reactive group for a neutral loss marker according to this invention (see FIG. 6a). Similarly, FIG. 6b shows a peptide tag that uses a derivative of O-methylisourea as the reactive functionality for the labelling of epsilon amino groups in lysine. In FIGS. 6a and 6b pairs of tags for the labelling of pairs of samples are shown.

Amino Labelling with Michael Reagents

Michael reagents have a number of properties that make them attractive for labelling amino reactions and have been used for this purpose (Friedman & Wall, 1966, J Org Chem 31: 2888-2894; Morpurgo et al., 1996, Bioconjug Chem (3): 363-368; Friedman & Finley, 1975, Int J Pept Protein Res 7 (6); Masri & Friedman, 1988, J Protein Chem 7: 49-54; Graham & Mechanic, 1986, Anal Biochem 153(2); Esterbauer et al., 1975, Z Naturforsch [C] 30 (4)).

A number of Michael reagents are relatively stable in aqueous solution and the structures of these compounds can be varied extensively to achieve different degrees of reactivity and selectivity. Reagents based on sulphones are generally more convenient and effective for labelling amino-groups than the more widely used esters. Michael reagents that have been used with proteins include compounds such as acrylonitrile, acrylamide, vinyl pyridine, methyl vinyl sulphone and methyl vinyl ketone. The reaction of these compounds have been compared (Friedman & Wall, 1966, above) and linear relationships between the reaction kinetics of these structurally similar compounds are observed. These linear relationships indicate that the reactions of this class of compounds take place by the same mechanism although their rates of reaction differ with the sulphone and ketone compounds found to be by far the most reactive.

The choice of a Michael reagent for the purposes of this invention is dependent on a number of criteria, included rates of reaction, chances of side-reactions apart from the Michael addition and ease of synthesis of different variants of the compound. Vinyl ketones can, for example, undergo other reactions besides Michael addition, particularly nucleophilic attack of the ketone after Michael addition has taken place. The ketone functionality can undergo this further reaction with a variety of nucleophiles, including the usual biological nucleophiles. Similarly, nitrile compounds can undergo hydrolysis of the nitrile functionality to the carboxylic acid, although typically this reaction will not occur under the conditions used in most biological assays. Alkenyl sulphones do not undergo reactions other than the Michael addition under the conditions used in typical biological assays. Alkenyl sulphones generally react rapidly with biological nucleophiles and there is an extensive literature on the synthesis of different forms of alkenyl sulphone. For these reasons alkenyl sulphones are preferred Michael reagents for use in the biological assays of this invention. Compounds such as N-ethylmaleimide also react rapidly with proteins by Michael addition and are reasonably stable under the conditions used for labelling proteins, although alkaline hydrolysis is observed when these reagents are polymer bound. Thus maleimide compounds are also preferred Michael reagents for use in the biological assays of this invention. In most circumstances nitrile reagents are also preferred reagents although a nitrile reagent will tend to react more slowly than corresponding sulphones. Similarly acrylamides react still more slowly. These preferences do not mean that the other Michael reagents available are unsuitable for this invention, but for most purposes rapid reaction of the reagents is preferred. Under appropriate conditions almost any of the Michael reagents could be used here.

A preferred class of lysine-selective reagents for use in this invention comprise hindered alkenyl sulphones as the lysine selective reactive groups. Combinations of these reagents under appropriate mild conditions can allow a high degree of discrimination between alpha-amino groups and lysine epsilon-amino groups in amine-labelling reactions. Vinyl sulphones are known to react readily with primary amines giving a di-alkylated product. These reagents will react more rapidly with epsilon-amino groups at high pHs (>9.0) than with alpha-amino groups but the discrimination of these unhindered sulphones is poor. More hindered alkenyl sulphones such as propenyl sulphones and butenyl sulphones show a greatly enhanced discrimination in favour of epsilon amino groups when compared with the vinyl sulphones. In addition, these hindered reagents produce the mono-alkylated product almost exclusively. Moreover, lysine epsilon-amino groups that have been mono-alkylated with some of the more hindered sulphones are resistant to further reaction with other amine reactive reagents.

FIG. 7 illustrates a pair of neutral loss markers that have been activated with a trifluoropropenyl sulphone functionality (Tsuge et al., 1995, J. Chem. Soc. Perkin Trans. 1: 2761-2766). This reagent will react once with a primary amine to form a secondary amino-group that can still be protonated. In FIG. 7, “MA” refers to Michael addition, while “CID” refers to collision induced dissociation.

Amine Labelling with Active Esters

Active esters are widely used to label amino groups. The labelling reaction produces an amide. In preferred embodiments of the invention, the overall charge of the labelled biomolecule should remain the same. However, conversion of an amino group to an amide will result in the loss of a readily protonated group in the labelled biomolecule and thus will reduce the charge, if the biomolecule is analysed in the positive ion mode.

FIG. 13 shows a tag that reacts with amino groups through the presence of an N-hydroxysuccinimide ester. The tag will undergo neutral loss but also contains a tertiary amino group to replace the amino group blocked by reaction of the tag. This tag can be produced by conventional peptide synthesis. The amino acid FMOC-piperazin-1-ylacetate is commercially available (Sigma-Aldrich, UK). Thus the sequence comprises (N)-Acetyl-alanine-piperazine-1-ylacetate-(C). This can be activated to an N-hydroxysuccinimide ester by methods well known in the art. In FIG. 13, step (1) shows coupling of the tags to sample peptides while step (2) shows analysis of peptides by low energy collision.

If the negative ion mode is to be used for the analysis of labelled biomolecules, then loss of positive charges on amino groups is not an issue. In this case, a variant of the tag in FIG. 13 could be used where the piperazinyl group is replaced with proline.

### Cysteine Labelling

FIG. 8 shows a pair of isobaric thiol-reactive markers according to this invention. The aspartic acid-proline sequence can be prepared by standard solid phase FMOC synthesis as discussed above. The C-terminal carboxylic acid group of the proline could be left unmodified but in FIG. 8 it is shown as an amide. This can be prepared by carrying out the synthesis of the asp-pro dipeptide using a rink amide (see U.S. Pat. No. 5,124,478) resin or a PAL resin. Finally the haloacetyl group may be introduced as described by Arar et al. (1995, Bioconjug Chem. 6(5): 573-577). FIG. 8 shows in step (1) the labelling of a thiol on a peptide from two samples. The tagged peptides will have the same mass-to-charge ratios since the tags are isobaric but, after the CID cleavage and neutral loss of part of the tag shown in step (2), the peptide ions that are left will now have different mass-to-charge ratios.

Maleimide compounds are also excellent reagents for thiol labelling and can be readily introduced into peptide tags according to the invention during solid phase synthesis (Marburg et al., 1996, Bioconjug Chem. 7(5):612-616). Maleimides can also react with amino groups as discussed above in relation to Michael reagents.

### Labelling and Preparation of Peptides for Mass Spectrometry

Various techniques are known in the art for the coupling of marker molecules to analytes such as peptides and polypeptides. These techniques are largely determined by the coupling reaction used, which is dependent, in turn, upon the reactive functionality present in the marker molecule and the reactive functionality on the peptide or polypeptide to which the marker will be coupled.

### Strategies for Analysis of Cysteine-Containing Peptides

A convenient method of labelling peptides exploits the high reactivity of the thiol function of cysteine residues. The thiol group can be labelled very selectively and rapidly under mild reaction conditions. Typically, maleimidyl or iodoacetyl reactive groups are introduced into the marker molecule as these are robust thiol-reactive groups. This approach is used in the ICAT method (Gygi et al., 1999, above), in which pairs of polypeptide samples are reacted with pairs of biotin isotopes. The biotin isotopes in the published ICAT methods usually employ iodoacetyl reactive groups. In a related approach, referred to as “covalent chromatography” (Wang & Regnier, 2001, J. Chromatogr. A 924(1-2): 345-357), cysteine-containing peptides are captured reversibly on a thiol reactive resin, which allows peptides without thiols to be washed away. The captured peptides are then released and tagged on their amino-groups with isotope tags. The general method involves isolation of the polypeptides from tissue, cleavage of the polypeptides with trypsin or Lys-C (Jekel et al, 1983, Anal Biochem. 134(2): 347-354) to generate smaller peptides that are readily analysed by mass spectrometry, followed by labelling of the peptides with cysteine-reactive biotin or a cysteine reactive resin. The biotin or resin allows the cysteine-containing peptides to be separated from unreacted peptides and peptides that do not contain cysteine. Since only a subset of the tryptic or Lys-C peptides from a sample of polypeptides will contain cysteine, this method results in the loss of some peptides from the analysis. This is normally acceptable, as most proteins have at least one cysteine containing peptide and can thus be identified. The reduction in complexity of the sample that results from the isolation of only cysteine-containing peptides is quite beneficial as the number of peptides that must be analysed is considerably reduced.

### Isolation of Peptides Containing Cysteine

In the context of the present invention, a thiol-labelling strategy is also useful for certain embodiments of the invention.

In an embodiment of the invention, a protocol for the analysis of a protein sample containing polypeptides with cysteine residues comprises the steps of:
1) cleaving the polypeptides with a sequence-specific endoprotease,
2) reducing and reacting all cysteine residues with a thiol-reactive affinity ligand neutral loss mass marker to form labelled peptides,
3) capturing labelled peptides onto an avidin derivatised solid support, and
4) analysing the captured labelled peptides by neutral loss mass spectrometry (as discussed above).

Protein samples may be digested with the sequence-specific endoprotease before or after reaction with the affinity ligand mass marker. The sequence-specific endoprotease may be Lys-C or trypsin. Similarly, reduction of cysteine residues may take place before or after digestion with the sequence-specific endoprotease.

Examples of cysteine reactive tags comprising the affinity ligand biotin are shown FIG. 9. The biotin tags shown in FIG. 9 are designed so that they can be synthesised by standard peptide synthesis procedures as discussed above. By changing the order of the sequence, the biotinylated lysine could be introduced between the aspartic acid and the iodacetyl group. This means that the biotin would remain linked to the peptide after CID cleavage of the tag. In an alternative approach, the lysine in group in the tags in FIG. 9 could be used to introduce additional isotopes into the tag.

In a further method suitable for isolating cysteine-containing peptides, a protocol for the analysis of a protein sample containing polypeptides with cysteine residues comprises the steps of:
1) cleaving the polypeptides with a sequence-specific endoprotease to form cleaved peptides,
2) reducing and reacting all cysteine residues in the cleaved peptides with a thiol-reactive affinity ligand,
3) coupling free amino groups in the cleaved peptides with an amine-reactive neutral loss mass marker to form labelled peptides,
4) capturing labelled peptides onto an avidin derivatised solid support, and
5) analysing the captured labelled peptides by neutral loss mass spectrometry (as discussed above).

In the above embodiment, the protein samples are digested with the sequence-specific endoprotease before reaction of the sample with the mass marker. The sequence-specific endoprotease may be Lys-C or trypsin. If Lys-C is used, tags will be able to react at both termini of the peptides.

### Blocking Thiol Groups

In the previous section, labelling of endogenous thiol groups in peptides and polypeptides was discussed. However, it may be useful to block thiol groups prior to any further analysis to prevent unwanted side-reactions and to avoid problems associated with disulphide bridges in polypeptides. This means that any cysteine disulphide bridges are reduced to free thiols and that the thiol moieties are capped prior to application of the methods of this invention. Since thiols are very much more reactive than the other side-chains in a protein this step can be achieved highly selectively.

A variety of reducing agents are known in the art for disulphide bond reduction. The choice of reagent may be determined on the basis of cost, or efficiency of reaction and compatibility with the reagents used for capping the thiols (for a review on these reagents and their use see Jocelyn, 1987, Methods Enzymol. 143: 246-256).

Typical capping reagents include N-ethylmaleimide, iodoacetamide, vinylpyridine, 4-nitrostyrene, methyl vinyl sulphone or ethyl vinyl sulphone (see for examples Krull et al., 1971, Anal. Biochem. 40(1): 80-85; Masri et al., 1972, Biochem Biophys. Res. Commun. 47(6): 1408-1413; Friedman et al., 1980, Anal. Biochem. 106(1): 27-34).

Typical reducing agents include mercaptoethanol, dithiothreitol (DTT), sodium borohydride and phosphines such as tributylphosphine (see Ruegg & Rudinger, 1977, Methods Enzymol. 47: 111-116) and tris(carboxyethyl)phosphine (Burns et al., 1991, J Org. Chem. 56: 2648-2650). Mercaptoethanol and DTT may be less suitable for use with thiol reactive capping reagents as these compounds contain thiols themselves.

It is worth noting that the reduction and thiol blocking may take place simultaneously: phosphine based reducing reagents are compatible with vinyl sulphone reagents (Masri & Friedman, 1988, above).

### Amine Labelling Strategies

### Terminal Peptides

In some embodiments, the markers of the invention are coupled at one or other terminus of the peptide that is to be analysed. There are some methods known in the art for achieving this. In addition, some new methods are introduced in this application.

Isolation of N- or C-terminal peptides has been described previously as methods to determine a global expression profile of a protein sample. Isolation of terminal peptides ensures that at least one and only one peptide per protein is isolated from a given polypeptide or peptide thus ensuring that the complexity of the sample that is analysed does not have more components than the original sample. Methods for isolating peptides from the termini of polypeptides are discussed in U.S. Pat. No. 5,470,703, WO98/32876, WO00/20870, WO02/099124 and WO02/099436. In particular, U.S. Pat. No. 5,470,703, WO98/32876 and WO02/099436 provide methods of isolating a C-terminal peptide from a polypeptide. These C-terminal methods generate peptides with a free amino group at the alpha-amino position that can be easily labelled with the Neutral Loss Mass Markers of this invention.

### Novel Peptide Sampling and Amine Labelling Methods

In further aspects of this invention, novel amine labelling methods are provided. These methods are based on the use of sequence-specific endoproteases that cleave polypeptides immediately C-terminal to Lysine residues. This results in peptides with an epsilon amino group at the C-terminus of each fragment peptide, except for the C-terminal peptide from the parent polypeptide, which may not have a C-terminal Lysine group. The cleavage reaction also leaves free alpha amino groups in the cleavage peptides, although the original N-terminal alpha-amino group of the parent polypeptide may be naturally blocked. This means that there are free amino groups at both ends of the majority of the cleavage fragments that would be generated by Lys-C cleavage, which can be easily labelled. The use of Lys-C in combination with amino labelling is an effective method for controlling the number of tags introduced into peptides for subsequent analysis.

In addition, the Lys-C fragments that are generated from the cleavage of larger polypeptides exist as two distinct populations: those fragments that contain arginine and those fragments that have no arginine. If these Lys-C fragments are labelled with an amino-reactive tag and then cleaved with Arg-C or trypsin a new fragment population is generated providing additional methods for analysing peptide mixtures. A number of distinct classes of peptides will result from this process of initial cleavage of a polypeptide mixture by Lys-C, amine-labelling with a tag and second cleavage with trypsin or Arg-C, which are illustrated by way of example in FIGS. 10a and 10b. In step (1) of FIG. 10a, polypeptides are cleaved with Lys-C. Step (2) of FIG. 10a shows labelling of cleaved peptides with amine reactive tags. Step (3) of FIG. 10b shows cleavage of peptides with Trypsin or Arg-C.

The different classes of resulting peptides are numbered in FIG. 10b and are described below:
1—Peptides that do not have an arginine group and which are labelled at both the alpha-amino group and the epsilon amino group after Lys-C cleavage.
2—Peptides that have a mass marker only at the epsilon amino group because they came from a labelled Lys-C cleavage fragment that contained an arginine group. The presence of the arginine groups means the labelled alpha amino group from the labelling of the Lys-C peptide is separated from the labelled epsilon amino group after cleavage with trypsin.
3—Peptides that have a mass marker at the alpha amino group and an arginine function at the C-terminus from Lys-C peptides with at least one arginine group.
4—Peptides that have an arginine group and a free alpha amino group, from a Lys-C peptide with two or more arginine groups.
5—Peptides with a mass marker at the alpha-amino position but no arginine or lysine at the C-terminus because they were derived from the C-terminus of the parent polypeptide after cleavage with Lys-C and where the Lys-C peptides have no arginine groups present.
6—Peptides with no mass marker at the alpha-amino position but no arginine or lysine at the C-terminus because they were derived from the C-terminus of the parent polypeptide after cleavage with Lys-C and the C-terminal peptides contain arginine. Subsequent cleavage of these arginine containing C-terminal peptides with trypsin or ArgC will remove any label at the alpha amino of the of the original Lys-C peptide.
7—Peptides with no mass marker at the alpha-amino position because they were naturally blocked in the parent polypeptide and a mass marker at the C-terminus because they are terminated by Lysine.
8—Peptides with no mass marker at the alpha-amino position because they were naturally blocked in the parent polypeptide and an arginine group at the C-terminus.

This labelling strategy has a number of advantages. Depending on the choice of tag, different peptide populations can be analysed. If the tag comprises an affinity ligand, distinct subsets of peptides can be isolated. This will be discussed in more detail below. A further advantage of the Lys-C, tagging, trypsin strategy is that the number of tags that are incorporated into peptides is controlled. Three distinct populations can be identified: peptides with no tag (classes 4, 6 and 8), peptides with only one tag (classes 2, 3, 5 and 7) and peptides with two tags (class 1). Different applications of this labelling strategy will now be discussed in more detail.

An additional class of peptides, not shown in FIGS. 10a and 10b, will be generated from peptides that comprise a lysine residue with a proline residue as the next C-terminal amino acid in the sequence. These peptides will not be cleaved by Lys-C or trypsin and will thus have a tag in the middle of the peptide (i.e. not at either terminus).

### Shotgun Analysis of Peptides

In the context of the use of neutral loss mass markers, cleavage of polypeptides with Lys-C followed by labelling and a second cleavage with trypsin is highly advantageous. Quantitative shotgun analysis of peptide mixtures is enabled in certain favoured embodiments of the labelling method, shown by way of example in FIGS. 10a and 10b, where the tag is a neutral loss tag according to this invention.

In these embodiments two or more samples of polypeptide mixtures are isolated and each cleaved with Lys-C in separate containers. Each sample of Lys-C cleavage peptides is then coupled to a distinct neutral loss mass marker, preferably of the form shown in FIG. 14. The tagged peptide samples are then pooled and treated with trypsin. The trypsin digest can then be analysed directly by a shotgun peptide sequencing method such as MudPIT. The untagged peptides (classes 4, 6 and 8 in FIG. 10b) will be sequenced as normal, while the tagged peptides will generate sequence spectra with split peaks. In the peptides with tags at the N-terminus, the split peaks will appear in the a, b and c ion series in the sequence spectra while in the peptides with tags at C-terminal epsilon amino groups, the split peaks will appear in the x, y and z series. The b and y series tend to predominate though so the split peaks will typically appear in these fragments. In peptides with a tag at both termini (class 1 peptides), the split peaks will appear in all of the fragment series.

The neutral loss mass marker may additionally comprise an affinity tag, such as biotin, that would allow the tagged peptides to be separated from the untagged peptides. This step would take place after cleavage of the tagged peptides with trypsin. This would however, require deactivation of trypsin after the cleavage step.

Isolation of Peptides from Classes 4, 6 and 8

As mentioned in the previous section describing shotgun analysis of tagged peptides generated according to the method exemplified in FIGS. 10a and 10b, it is possible to separate tagged peptides from untagged peptides if the tag comprises an affinity ligand. It is thus possible to isolate the untagged peptides if all of the tagged peptides are captured using the affinity ligand. This would leave the peptides from classes 4, 6 and 8 in solution. Alternatively, the Lys-C peptides can be captured onto an amine-reactive resin or to an amine reactive biotin compound. Peptides in classes 4, 6 and 8 can be cleaved from the resin or biotin with trypsin. The peptides in classes 4 and 6 will have free alpha amino groups, which means that these classes of peptides, once isolated, can be tagged with neutral loss markers for further analysis. To enable quantitative analysis of two or more samples of polypeptides with this approach would require that the steps of Lys-C cleavage, affinity tagging or resin capture and trypsin cleavage would all have to take place on each sample separately. After isolation of the peptides in classes 4, 6 and 8, the peptide samples would be tagged with uniquely identifiable neutral loss mass markers and then would be pooled for analysis by LC-MS/MS, using a shotgun analysis technique like MudPIT if desired.

Isolation of Peptide from Classes 3 and 5

In certain embodiments of the labelling strategy depicted by way of example in FIGS. 10a and 10b, it is possible to separate peptides in classes 3 and 5 from peptides in classes 1, 2, or 7. If the tag comprises an affinity ligand in which the reactive functionality is an isothiocyanate or a related compound that reacts with the alpha-amino group to form a hydantoin structure, then after capture of the tagged peptides it will be possible to selectively cleave peptides in classes 3 and 5 from the solid support onto which the tagged peptides are captured. The cleavage step typically requires acidic conditions and will leave the peptides in classes 3 and 5 in solution with free alpha amino groups although one amino acid will be lost from the N-terminus of these peptides in the hydantoin cleavage reaction. These peptides can then be labelled with a Neutral Loss Tag for further analysis.

To enable quantitative analysis of two or more samples of polypeptides with this approach would require that the steps of Lys-C cleavage, affinity tagging or resin capture, trypsin cleavage and hydantoin cleavage would all have to take place on each sample separately. After isolation of the peptides in classes 3 and 5, the peptide samples would be tagged with uniquely identifiable Neutral Loss Mass Markers and then would be pooled for analysis by LC-MS/MS, using a shotgun analysis technique like MudPIT if desired.

A similar effect can be achieved by reacting the Lys-C cleavage peptides with a suitable reactive resin such as diisothiocyanato-glass (DITC-glass). The Lys-C peptides on the glass can be treated with trypsin to cleave the peptides followed by treatment under acid conditions to cleave the class 3 and 5 peptides from the glass.

### Post-Translational Modifications

### Isolation of Carbohydrate-Modified Proteins

Carbohydrates are often present as a post-translational modification of polypeptides. Various affinity chromatography techniques for the isolation of polypeptides with these modifications are known (for review, see Gerard, 1990, Methods Enzymol 182: 529-539). A variety of natural ligands that bind specifically to carbohydrates are known. The members of a well-known class of protein receptors, known as lectins, are highly selective for particular carbohydrate functionalities. Affinity columns derivatised with specific lectins can be used to isolate proteins with particular carbohydrate modifications, whilst affinity columns comprising a variety of different lectins could be used to isolate populations of proteins with a variety of different carbohydrate modifications.

In one embodiment of the invention, a protocol for the analysis of a protein sample comprising carbohydrate-modified proteins comprises the steps of:
1) treating the protein sample with a sequence-specific cleavage reagent (for example, trypsin or Lys-C) to form peptides,
2) passing the peptides sample through an affinity column containing lectins or boronic acid derivatives to capture carbohydrate-modified peptides,
3) labelling the captured carbohydrate-modified peptides at their free alpha amino group generated by the sequence-specific cleavage with a neutral loss mass markers to form labelled peptides, and
4) analysing the labelled peptides by neutral loss mass spectrometry.

An N-hydroxysuccinimide activated marker could be used to label the free alpha-amino groups. If Lys-C is used then each carbohydrate-modified peptide will have a free epsilon-amino group as well as a free alpha amino group, both of which can be tagged.

Many carbohydrates have vicinal-diol groups present, i.e. hydroxyl groups present on adjacent carbon atoms. Diol containing carbohydrates that contain vicinal diols in a 1,2-cis-diol configuration will react with boronic acid derivatives to form cyclic esters. This reaction is favoured at basic pH but is easily reversed at acid pH. Resin immobilised derivatives of phenyl boronic acid have been used as ligands for affinity capture of proteins with cis-diol containing carbohydrates. In one embodiment of this invention a set of affinity ligand (peptide) mass markers comprising biotin linked to a phenylboronic acid entity could be synthesised. These boronic acid markers could used to label two separate samples comprising peptides or proteins with carbohydrate modifications that contain vicinal cis-diols.

In another embodiment of the invention, a protocol for the analysis of a sample such as a protein sample containing carbohydrate-modified polypeptides comprises the steps of:
1) reacting the sample at basic pH with a boronic acid affinity ligand neutral loss mass marker to form labelled polypeptides,
2) cleaving the labelled polypeptides with a sequence-specific endoprotease to form labelled peptides,
3) capturing labelled peptides onto an avidin derivatised solid support, and
4) analysing the captured labelled peptides by neutral loss mass spectrometry.

The sample may be digested with the sequence-specific endoprotease before or after reaction of the sample with the affinity ligand mass marker.

Vicinal-diols, in sialic acids for example, can also be converted into carbonyl groups by oxidative cleavage with periodate. Enzymatic oxidation of sugars containing terminal galactose or galactosamine with galactose oxidase can also convert hydroxyl groups in these sugars to carbonyl groups. Complex carbohydrates can also be treated with carbohydrate cleavage enzymes, such as neuramidase, which selectively remove specific sugar modifications leaving behind sugars, which can be oxidised. These carbonyl groups can be tagged allowing proteins bearing such modifications to be detected or isolated. Hydrazide reagents, such as Biocytin hydrazide (Pierce & Warriner Ltd, Chester, UK) will react with carbonyl groups in carbonyl-containing carbohydrate species (Bayer et al., 1988, Anal. Biochem. 170: 271-281). Alternatively a carbonyl group can be tagged with an amine modified biotin, such as Biocytin and EZ-Link™ PEO-Biotin (Pierce & Warriner Ltd, Chester, UK), using reductive alkylation (Means, 1977, Methods Enzymol 47: 469-478; Rayment, 1997, Methods Enzymol 276: 171-179). Proteins bearing vicinal-diol containing carbohydrate modifications in a complex mixture can thus be biotinylated. Biotinylated, hence carbohydrate-modified, proteins may then be isolated using an avidinated solid support.

A set of peptide mass markers according to this invention can synthesised for the analysis of carbohydrate-modified peptides that have been oxidised with periodate, as shown by way of example in FIG. 11. FIG. 11 shows a set of two markers derived from aspartic acid and proline. Different isotopically substituted forms of proline would be used to prepare the two different markers. The total mass of each of the two markers is the same but the proline in each marker differs from the other marker by five Daltons.

A further embodiment of the invention for the analysis of a sample such as a protein sample containing carbohydrate-modified polypeptides comprises the steps of:
1) treating the sample with periodate to allow carbohydrates with vicinal cis-diols on glycopeptides to gain a carbonyl functionality,
2) labelling the carbonyl functionality with a hydrazide activated neutral loss peptide mass marker (for example, as shown in FIG. 11) to form labelled polypeptides,
3) digesting the labelled polypeptides with a sequence-specific endoprotease to form labelled peptides
4) analysing the labelled peptides by neutral loss mass spectrometry.

The protein sample may be digested with the sequence-specific endoprotease before or after reaction of the sample with the neutral loss mass marker.

### Isolation of Phosphopeptides

Phosphorylation is a ubiquitous reversible post-translational modification that appears in the majority of signalling pathways of almost all organisms as phosphorylation is widely used as a transient signal to mediate changes in the state of individual proteins. It is an important area of research and tools which allow the analysis of the dynamics of phosphorylation are essential to a full understanding of how cells responds to stimuli, which includes the responses of cells to drugs.

Techniques for the analysis of phosphoserine and phosphothreonine containing peptides are well known. One class of such methods is based on a well-known reaction for beta-elimination of phosphates. This reaction results in phosphoserine and phosphothreonine forming dehydroalanine and methyldehydroalanine, both of which are Michael acceptors and will react with thiols. This has been used to introduce hydrophobic groups for affinity chromatography (see for example Holmes, 1987, FEBS Lett 215(1): 21-24). Dithiol linkers have also been used to introduce fluorescein and biotin into phosphoserine and phosphothreonine containing peptides (Fadden & Haystead, 1995, Anal Biochem 225(1): 81-88; Yoshida et al., 2001, Nature Biotech 19: 379-382). The method of Yoshida et al. for affinity enrichment of proteins phosphorylated at serine and threonine could be improved by using an iodoacetyl marker as shown by way of example in FIG. 9 to allow the comparison of multiple samples. This would be particularly useful for the analysis of the dynamics of phosphorylation cascades.

A marker peptide of the form shown in FIG. 12 would allow direct labelling of beta-eliminated phosphothreonine and phosphoserine residues without a dithiol linker. The marker dipeptide of FIG. 12 is derived from aspartic acid and proline. Different isotopically substituted forms of proline are used to prepare the two different markers. The mass each marker differs from the other marker by five Daltons. The mercaptopropionic acid residue provides a free thiol, which can nucleophilically attack dehydroalanine and methyldehydroalanine. An improved protocol for the beta-elimination based labelling procedure is known. This improved procedure involves barium catalysis (Byford, 1991, Biochem J. 280: 261-261). This catalysis makes the reaction 20-fold faster reducing side-reactions to undetectable levels. The marker peptide shown in FIG. 12 could be easily coupled to dehydroalanine or methyldehydroalanine generated from beta-elimination of phosphates using barium catalysis.

Thus in a further embodiment of the invention, a sample such as protein sample comprising polypeptides phosphorylated at serine and threonine may be analysed in a method comprising the steps of:
1) treating the sample with barium hydroxide to beta-eliminate phosphate groups from phosphoserine and phosphothreonine,
2) labelling the resultant dehydroalanine or methyldehydroalanine functionalities with a thiol activated neutral loss peptide mass marker (for example, as shown in FIG. 12) to form biotinylated labelled polypeptides,
3) digesting the biotinylated labelled polypeptides with a sequence-specific endoprotease to form biotinylated labelled peptides,
4) analysing the biotinylated labelled peptides by neutral loss mass spectrometry.

The sample may be digested with the sequence-specific endoprotease before or after reaction of the sample with the neutral loss mass marker.

A number of research groups have reported on the production of antibodies, which bind to phosphotyrosine residues in a wide variety of proteins (see for example Frackelton et al., 1991, Methods Enzymol 201: 79-92, and other articles in that issue of Methods Enzymol.). This means that a significant proportion of proteins that have been post-translationally modified by tyrosine phosphorylation may be isolated by affinity chromatography using these antibodies as the affinity column ligand.

These phosphotyrosine binding antibodies can be used in the context of this invention to isolate terminal peptides from proteins containing phosphotyrosine residues. The tyrosine-phosphorylated proteins in a complex mixture may be isolated using anti-phosphotyrosine antibody affinity columns.

In a further embodiment of the invention, a method for the analysis of a sample of proteins comprising polypeptides phosphorylated at tyrosine comprises the steps of:
1) treating the protein sample with a sequence-specific cleavage reagent (such as for example trypsin or Lys-C) to form peptides,
2) passing the peptides through an affinity column containing anti-phosphotyrosine antibodies to capture phosphotyrosine-modified peptides,
3) labelling the captured phosphotyrosine-modified peptides at their free amino groups generated by the sequence-specific cleavage using a neutral loss mass markers to form labelled peptides, and
4) analysing the labelled peptides by neutral loss mass spectrometry.

### Analysis of Peptides by Mass Spectrometry

Key features of a mass spectrometer are as follows:

### Inlet System->Ion Source->Mass Analyser->Ion Detector->Data Capture System.

There are preferred inlet systems, ion sources and mass analysers for the purposes of analysing peptides.

### Inlet Systems

For the invention, a chromatographic or electrophoretic separation can be used to reduce the complexity of the sample prior to analysis by mass spectrometry. A variety of mass spectrometry techniques are compatible with separation technologies particularly capillary zone electrophoresis and high performance liquid chromatography (HPLC). The choice of ionisation source is limited to some extent if a separation is required as ionisation techniques such as MALDI and FAB (discussed below) which ablate material from a solid surface are less suited to chromatographic separations. For most purposes, it has been very costly to link a chromatographic separation in-line with mass spectrometric analysis by one of these techniques. Dynamic FAB and ionisation techniques based on spraying such as electrospray, thermospray and APCI are all readily compatible with in-line chromatographic separations and equipment to perform such liquid chromatography mass spectrometry analysis is commercially available.

### Ionisation Techniques

For many biological mass spectrometry applications so called “soft” ionisation techniques are used. These allow large molecules such as proteins and nucleic acids to be ionised essentially intact. The liquid phase techniques allow large biomolecules to enter the mass spectrometer in solutions with mild pH and at low concentrations. A number of techniques are appropriate for use with the present invention including but not limited to Electrospray Ionisation Mass Spectrometry (ESI-MS), Fast Atom Bombardment (FAB), Matrix Assisted Laser Desorption Ionisation Mass Spectrometry (MALDI MS) and Atmospheric Pressure Chemical Ionisation Mass Spectrometry (APCI-MS).

### Electrospray Ionisation

Electrospray ionisation requires that the dilute solution of the analyte molecule is ‘atomised’ into the spectrometer, i.e. injected as a fine spray. The solution is, for example, sprayed from the tip of a charged needle in a stream of dry nitrogen and an electrostatic field. The mechanism of ionisation is not fully understood but is thought to work broadly as follows. In a stream of nitrogen the solvent is evaporated. With a small droplet, this results in concentration of the analyte molecule. Given that most biomolecules have a net charge this increases the electrostatic repulsion of the dissolved molecule. As evaporation continues this repulsion ultimately becomes greater than the surface tension of the droplet and the droplet disintegrates into smaller droplets. This process is sometimes referred to as a ‘Coulombic explosion’. The electrostatic field helps to further overcome the surface tension of the droplets and assists in the spraying process. The evaporation continues from the smaller droplets which, in turn, explode iteratively until essentially the biomolecules are in the vapour phase, as is all the solvent. This technique is of particular importance in the use of mass labels in that the technique imparts a relatively small amount of energy to ions in the ionisation process and the energy distribution within a population tends to fall in a narrower range when compared with other techniques. The ions are accelerated out of the ionisation chamber by the use of electric fields that are set up by appropriately positioned electrodes. The polarity of the fields may be altered to extract either negative or positive ions. The potential difference between these electrodes determines whether positive or negative ions pass into the mass analyser and also the kinetic energy with which these ions enter the mass spectrometer. This is of significance when considering fragmentation of ions in the mass spectrometer. The more energy imparted to a population of ions the more likely it is that fragmentation will occur through collision of analyte molecules with the bath gas present in the source. By adjusting the electric field used to accelerate ions from the ionisation chamber it is possible to control the fragmentation of ions. This is advantageous when fragmentation of ions is to be used as a means of removing tags from a labelled biomolecule. Electrospray ionisation is particularly advantageous as it can be used in-line with liquid chromatography, referred to as Liquid Chromatography Mass Spectrometry (LC-MS).

### Matrix Assisted Laser Desorption Ionisation (MALDI)

MALDI requires that the biomolecule solution be embedded in a large molar excess of a photo-excitable ‘matrix’. The application of laser light of the appropriate frequency results in the excitation of the matrix which in turn leads to rapid evaporation of the matrix along with its entrapped biomolecule. Proton transfer from the acidic matrix to the biomolecule gives rise to protonated forms of the biomolecule which can be detected by positive ion mass spectrometry, particularly by Time of Flight (TOF) mass spectrometry. Negative ion mass spectrometry is also possible by MALDI TOF. This technique imparts a significant quantity of translational energy to ions, but tends not to induce excessive fragmentation despite this. Accelerating voltages can again be used to control fragmentation with this technique though.

### Fast Atom Bombardment

Fast Atom Bombardment has come to describe a number of techniques for vaporising and ionising relatively involatile molecules. The essential principal of these techniques is that samples are desorbed from surfaces by collision of the sample with accelerated atoms or ions, usually xenon atoms or caesium ions. The samples may be coated onto a solid surface as for MALDI but without the requirement of complex matrices. These techniques are also compatible with liquid phase inlet systems—the liquid eluting from a capillary electrophoresis inlet or a high pressure liquid chromatography system pass through a frit, essentially coating the surface of the frit with analyte solution which can be ionised from the frit surface by atom bombardment.

### Mass Analysers

Fragmentation of peptides by collision induced dissociation is used in this invention to identify tagged peptides or proteins. Various mass analyser geometries may be used to fragment peptides and to determine the mass of the fragments.

### MS/MS and MSⁿ Analysis of Peptides

Tandem mass spectrometers allow ions with a pre-determined mass-to-charge ratio to be selected and fragmented by collision induced dissociation (CID). The fragments can then be detected providing structural information about the selected ion. When peptides are analysed by CID in a tandem mass spectrometer, characteristic cleavage patterns are observed, which allow the sequence of the peptide to be determined. Natural peptides typically fragment randomly at the amide bonds of the peptide backbone to give series of ions that are characteristic of the peptide. In the structure (I) shown below, CID fragment series are denoted aₙ, bₙ, cₙ, etc. for cleavage at the n^{th} peptide bond where the charge of the ion is retained on the N-terminal fragment of the ion. Similarly, fragment series are denoted xₙ, yₙ, zₙ, etc. where the charge is retained on the C-terminal fragment of the ion.

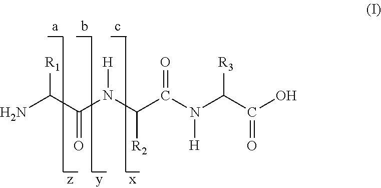

Trypsin and thrombin are useful cleavage agents for tandem mass spectrometry as they produce peptides with basic groups at both ends of the molecule, i.e. the alpha-amino group at the N-terminus and lysine or arginine side-chains at the C-terminus. This favours the formation of doubly charged ions, in which the charged centres are at opposite termini of the molecule. These doubly charged ions produce both C-terminal and N-terminal ion series after CID. This assists in determining the sequence of the peptide. Generally speaking only one or two of the possible ion series are observed in the CID spectra of a given peptide. In low-energy collisions typical of quadrupole based instruments the b-series of N-terminal fragments or the y-series of C-terminal fragments predominate. If doubly charged ions are analysed then both series are often detected. In general, the y-series ions predominate over the b-series.

A typical tandem mass spectrometer geometry is a triple quadrupole, which comprises two quadrupole mass analysers separated by a collision chamber, also a quadrupole. This collision quadrupole acts as an ion guide between the two mass analyser quadrupoles. A gas can be introduced into the collision quadrupole to allow collision with the ion stream from the first mass analyser. The first mass analyser selects ions on the basis of their mass/charge ratio, which pass through the collision cell where they fragment. The fragment ions are separated and detected in the third quadrupole. Induced cleavage can be performed in geometries other than tandem analysers. Ion traps mass spectrometers can promote fragmentation through introduction of a gas into the trap itself with which trapped ions will collide. Ion traps generally contain a bath gas, such as helium but addition of neon for example, promotes fragmentation. Similarly photon induced fragmentation could be applied to trapped ions. Another favourable geometry is a Quadrupole/Orthogonal Time of Flight tandem instrument where the high scanning rate of a quadrupole is coupled to the greater sensitivity of a reflectron TOF mass analyser to identify the products of fragmentation.

Conventional ‘sector’ instruments are another common geometry used in tandem mass spectrometry. A sector mass analyser comprises two separate ‘sectors’, an electric sector which focuses an ion beam leaving a source into a stream of ions with the same kinetic energy using electric fields. The magnetic sector separates the ions on the basis of their mass to generate a spectrum at a detector. For tandem mass spectrometry a two sector mass analyser of this kind can be used where the electric sector provide the first mass analyser stage, the magnetic sector provides the second mass analyser, with a collision cell placed between the two sectors. Two complete sector mass analysers separated by a collision cell can also be used for analysis of mass tagged peptides.

### Ion Traps

Ion Trap mass analysers are related to the quadrupole mass analysers. The ion trap generally has a 3 electrode construction—a cylindrical electrode with ‘cap’ electrodes at each end forming a cavity. A sinusoidal radio frequency potential is applied to the cylindrical electrode while the cap electrodes are biased with DC or AC potentials. Ions injected into the cavity are constrained to a stable circular trajectory by the oscillating electric field of the cylindrical electrode. However, for a given amplitude of the oscillating potential, certain ions will have an unstable trajectory and will be ejected from the trap. A sample of ions injected into the trap can be sequentially ejected from the trap according to their mass/charge ratio by altering the oscillating radio frequency potential. The ejected ions can then be detected allowing a mass spectrum to be produced.

Ion traps are generally operated with a small quantity of a ‘bath gas’, such as helium, present in the ion trap cavity. This increases both the resolution and the sensitivity of the device as the ions entering the trap are essentially cooled to the ambient temperature of the bath gas through collision with the bath gas. Collisions both increase ionisation when a sample is introduced into the trap and dampen the amplitude and velocity of ion trajectories keeping them nearer the centre of the trap. This means that when the oscillating potential is changed, ions whose trajectories become unstable gain energy more rapidly, relative to the damped circulating ions and exit the trap in a tighter bunch giving a narrower larger peaks.

Ion traps can mimic tandem mass spectrometer geometries, in fact they can mimic multiple mass spectrometer geometries allowing complex analyses of trapped ions. A single mass species from a sample can be retained in a trap, i.e. all other species can be ejected and then the retained species can be carefully excited by super-imposing a second oscillating frequency on the first. The excited ions will then collide with the bath gas and will fragment if sufficiently excited. The fragments can then be analysed further. It is possible to retain a fragment ion for further analysis by ejecting other ions and then exciting the fragment ion to fragment. This process can be repeated for as long as sufficient sample exists to permit further analysis. It should be noted that these instruments generally retain a high proportion of fragment ions after induced fragmentation. These instruments and FTICR mass spectrometers (discussed below) represent a form of temporally resolved tandem mass spectrometry rather than spatially resolved tandem mass spectrometry which is found in linear mass spectrometers.

### Fourier Transform Ion Cyclotron Resonance Mass Spectrometry (FTICR MS)

FTICR mass spectrometry has similar features to ion traps in that a sample of ions is retained within a cavity but in FTICR MS the ions are trapped in a high vacuum chamber by crossed electric and magnetic fields. The electric field is generated by a pair of plate electrodes that form two sides of a box. The box is contained in the field of a superconducting magnet which in conjunction with the two plates, the trapping plates, constrain injected ions to a circular trajectory between the trapping plates, perpendicular to the applied magnetic field. The ions are excited to larger orbits by applying a radio-frequency pulse to two ‘transmitter plates’ which form two further opposing sides of the box. The cycloidal motion of the ions generate corresponding electric fields in the remaining two opposing sides of the box which comprise the ‘receiver plates’. The excitation pulses excite ions to larger orbits which decay as the coherent motions of the ions is lost through collisions. The corresponding signals detected by the receiver plates are converted to a mass spectrum by Fourier Transform (FT) analysis.

For induced fragmentation experiments these instruments can perform in a similar manner to an ion trap—all ions except a single species of interest can be ejected from the trap. A collision gas can be introduced into the trap and fragmentation can be induced. The fragment ions can be subsequently analysed. Generally fragmentation products and bath gas combine to give poor resolution if analysed by FT analysis of signals detected by the ‘receiver plates’, however the fragment ions can be ejected from the cavity and analysed in a tandem configuration with a quadrupole, for example.

### Separation of Labelled Peptides by Chromatography or Electrophoresis

In various aspects of this invention, labelled biomolecules are subjected to a chromatographic separation prior to analysis by mass spectrometry. This is preferably high performance liquid chromatography (HPLC), which can be coupled directly to a mass spectrometer for in-line analysis of the peptides as they elute from the chromatographic column. A variety of separation techniques may be performed by HPLC but reverse phase chromatography is a popular method for the separation of peptides prior to mass spectrometry. Capillary zone electrophoresis is another separation method that may be coupled directly to a mass spectrometer for automatic analysis of eluting samples. These and other fractionation techniques may be applied to reduce the complexity of a mixture of biomolecules prior to analysis by mass spectrometry.

### Further Applications of Neutral Loss Markers

One application of the present invention is differential expression profiling of samples comprising complex mixtures of polypeptides. An example of this would be the comparison of the proteins present in a sample of cancer tissue compared with the corresponding normal undiseased or healthy tissue from the same host. In this situation, the proteins in both samples would be separately extracted using methods known in the art. The protein extracts are typically then treated to reduce disulfides, which are then capped as discussed above. The reduced and capped proteins are then digested by trypsin.

In conventional prior art approaches, the tryptic digests would be analysed by multidimensional chromatography followed by in-line electrospray ionisation mass spectrometry with “shotgun” sequencing of peptide ions that are produced. In shotgun sequencing methods, a mixture of peptides is sprayed into a mass spectrometer, usually as a fraction eluting from a chromatographic separation. The mass spectrometer is programmed to analyse the mixture in the MS-mode to detect ions and select ions for subsequent sequencing. A typical selection strategy is to simply select the three ions with the highest intensity where the ions must also exceed a specific m/z threshold and must also be different from the ions analysed in the last cycle (or different from the last two, three or more cycles) of analysis. Thus a relatively arbitrary subset of the ions that are present in a sample will be analysed. The present invention differs from convention approaches as described above.

The following abbreviations are used in the examples below:

### TFA: Trifluoroacetic Acid

### DMF: Dimethylformamide

### DCM: Dichloromethane

EDC: 1-Ethyl-3-(3-dimethylaminopropyl)carbodiimide

### TIS: Triisopropylsilane

### FMOC: FluorenylMethylOxyCarbonyl

HPLC: High performance Liquid Chromatography.

### EXAMPLE 1

### Synthesis of an Isobaric Pair of Active Ester Neutral Loss Tags

FIG. 14 illustrates a pair of isobaric tags. The peptide sequence N-terminus: Acetate-Alanine-Piperazin-1-yl Acetate-Beta Alanine-C-terminus is synthesised by standard FMOC solid phase peptide synthesis procedures. The beta-alanine carboxylic acid can be activated to form the N-hydroxysuccinimide ester by coupling N-hydroxysuccinimide in the presence of a suitable carbodiimide such as dicyclohexylcarbodiimide (DIC). In a typical coupling reaction the peptide would be dissolved with a small excess of DIC in a suitable solvent (DMF or ethyl acetate) and a small molar excess of N-hydroxysuccinimide is then added to the reaction. The reaction is then left for 1 to 2 hours at room temperature.

FIG. 14 shows in step (1) the coupling of this neutral loss tag pair to a peptide via an amino group. These sorts of tag peptides will be suitable for labelling of peptides as an alternative to TMT or iTRAQ reagents in quantitative shotgun peptidomics (Thompson et al., 2003, above; Ross et al., 2004, above; Dayon et al., 2008, Anal. Chem. 80(8): 2921-2931). In step (2) of FIG. 14, analysis of peptides by low energy collision is shown.

FIG. 15 illustrates the expected mass-to-charge ratios of the y-series from the MS/MS sequencing of this neutral loss tag peptide pair coupled to a short peptide (acetyl-AFLDASK [SEQ ID NO: 5]). This is different from the expected masses for a TMT reagent or and iTRAQ reagent. With the TMT approach, if a pair of isobaric TMT tags is coupled to two different samples of digested peptides, a pair of reporter ions derived from the tag will appear in the CID spectrum for each peptide pair that is selected for CID sequencing of those peptides. In contrast, the tags of this invention are not detected directly but quantification of a pair of peptides is possible because neutral loss of part of the tag results in mass shifted sequence ions in the CID spectrum. In this case, the tag is shown coupled to the epsilon amino group and the relative quantities of the two labelled peptides is determined by the ratio of the pairs of mass shifted y-series ions.

### EXAMPLE 2

### Synthesis of Isourea-Based Neutral Loss Tags

FIG. 16a illustrates a schematic of a synthetic protocol for the production of isourea reagents according to an embodiment of this invention. The peptide (N-terminus-acetate-aspartic acid-proline-lysine-amide-C-terminus) is synthesised by standard FMOC procedures using a PAL resin to produce the C-terminal amide. The peptide is purified, preferably by HPLC, and dried down in a vacuum desiccator. The epsilon amino of the lysine is then coupled with N,N′-Disuccinimidyl carbonate (SigmaAldrich) in DMF or as described (Morpurgo et al., 1999, J Biochem Biophys Methods. 38(1): 17-28).

The resulting N-hydroxysuccinimidyl carbamate is then reacted with aqueous ammonia to give the corresponding peptide urea. The peptide urea is converted to the corresponding isourea by addition to a stirred solution of dimethyl sulphate or alternatively, dropwise addition of dimethyl sulphate into a stirred solution of the peptide urea with cooling (U.S. Pat. No. 6,093,848; US2007/0015233A1; Ongley, 1947, Transactions of the Royal Society of New Zealand 77(1): 10-12).

In one embodiment, the conversion of the epsilon amino to a urea takes place on-column prior to peptide cleavage from the resin.

In an alternative embodiment, the epsilon amino of the lysine is coupled with phosgene. The carbamyl chloride is then reacted with ammonia to give the corresponding peptide urea. The peptide urea is converted to the corresponding isourea by reaction with dimethyl sulphate

The resulting reagent is a guanidination reagent that can be coupled to epsilon amino groups in polypeptides and peptides with high specificity and yield (Beardsley & Reilly, 2002, Anal Chem. 74(8): 1884-1890).

A related reagent that fragments less easily can be prepared by replacing the aspartic acid in this peptide structure with glutamic acid. Similarly, an even more stable structure can be prepared by substituting the aspartic acid with an alanine, valine, leucine or isoleucine residue.

A further related structure, shown in FIG. 16b, can be prepared by coupling the epsilon amino group of the peptide shown with N-Succinimidyl N-methylcarbamate (or methyl isocyanate as an alternative) to give the corresponding urea that can then be reacted with dimethyl sulphate as described above. The preparation of the urea can take place on-column during peptide synthesis.

Different isotopic variants of these peptide tag structures can be prepared by substituting stable isotope variants of the components of the peptide. For example, a pair of isobaric neutral loss tags can be prepared by preparing a first peptide with ¹³C₄, ¹⁵N aspartic acid (Cambridge Isotope Laboratories, Inc; Andover, Mass., USA) and ¹³C-acetic anhydride (SigmaAldrich) and a second peptide with ¹³C₅, ¹⁵N Proline (Cambridge Isotope Laboratories, Inc; Andover, Mass., USA). This is possible for all the structures discussed in this application and numerous isotopic variations should be apparent to one of skill in the art.

### EXAMPLE 3

### Synthesis of an Azide Modified Neutral Loss Tag Peptide

FIG. 17 part 1 illustrates an azide modified neutral loss tag reagent according to this invention. The peptide (N-terminus: acetate-aspartic acid-proline-lysine-amide-C-terminus) is synthesised by standard FMOC procedures using a Rink Amide resin to produce the C-terminal amide. The lysine residue is protected with an orthogonal protecting group such as the DDE group, i.e. N-alpha-Fmoc-N-epsilon-1-(4,4-dimethyl-2,6-dioxocyclohex-1-ylidene)ethyl-L-lysine (α-FMOC-ε-Dde-Lys; IRIS Biotech GmbH, Marktredwitz, Germany). In the synthesis of this peptide, the DDE group is removed with 2% hydrazine in DMF (Bycroft et al., 1993, J. Chem. Soc., Chem. Commun. 778) after completion of the main peptide structure. The resulting free epsilon amino group can then be coupled to azidopentanoic acid (Bachem, Bubendorf, Switzerland) using standard coupling procedures (Jagasia et al., 2009, J. Org. Chem. 74(8): 2964-2974). Cleavage and deprotection is preferably effected by reaction with TFA/DCM/TIS (10:85:5) for 30 min (Gogoi et al., 2007, Nucleic Acids Res. 35(21): e139).

### EXAMPLE 4

### Synthesis of Propynyl Isourea

FIG. 17 part 2 illustrates a schematic of a synthetic protocol for the production of a propynyl isourea reagent. The amino group of the propargylamine (Sigma) is coupled with N,N′-Disuccinimidyl carbonate (SigmaAldrich) as described in Example 2. The resulting N-hydroxysuccinimidyl carbamate is then reacted with aqueous ammonia to give the corresponding peptide urea. The peptide urea is converted to the corresponding isourea by reaction with dimethyl sulphate as described in Example 2. As discussed in Example 2, phosgene can be used in the preparation of the urea or an analogous urea compound can be prepared by coupling propargylamine with N-Succinimidyl N-methylcarbamate or methyl isocyanate.

### EXAMPLE 5

### Synthesis of Hexynyl Isourea

FIG. 18 part 2 illustrates a schematic of a synthetic protocol for the production of a propyne-linked isourea reagent. The 5-hexynoic acid (SigmaAldrich) and O-methylisourea sulphate (SigmaAldrich) are dissolved with diisopropylethylamine and EDC in DMF in a ratio of (1:1:1:2) respectively (see US 2006/0234314A1 for examples of this kind of coupling). The reaction is left overnight with stirring. The product is dried down with a rotary evaporator under vacuum at 35 degrees centigrade. The crude product can be purified by silica gel column chromatography.

### EXAMPLE 6

### Alternative Synthesis of Isourea-Based Neutral Loss Tags

FIG. 17 also shows a synthetic protocol for the production of isourea reagents according to an embodiment of this invention. The azide-modified peptide from Example 3 (as shown in FIG. 17 part 1) is coupled to the propynyl-containing isourea reagent from Example 4 (shown in FIG. 17 part 2) using the copper-catalysed azide alkyne cycloaddition (CuAAC) reaction (Gogoi et al., 2007, above). Typical coupling conditions involve dissolving 1 equivalent of the azide component with 3 equivalents of the alkynyl component in 50:50 water:tert butanol in the presence of 1 equivalent of Copper Sulphate and 4 equivalents of sodium ascorbate. The reaction is stirred at room temperature for 2 hours.

The peptide is purified, preferably by HPLC, and dried down in a vacuum desiccator. The resulting reagent is a guanidination reagent that can be coupled to epsilon amino groups in polypeptides and peptides with high specificity and yield as discussed above.

### EXAMPLE 7

### Alternative Synthesis of Isourea-Based Neutral Loss Tags

FIG. 18 also shows a synthetic protocol for the production of isourea reagents according to an embodiment of this invention. The azide-modified peptide from Example 3 (see FIG. 18 part 1) is coupled to the hexynyl-containing isourea reagent from Example 5 (see FIG. 18 part 2) using the copper-catalysed azide alkyne cycloaddition (CuAAC) reaction (Gogoi et al., 2007, above). Typical coupling conditions involve dissolving 1 equivalent of the azide component with 3 equivalents of the alkynyl component in 50:50 water:tert butanol in the presence of 1 equivalent of Copper Sulphate and 4 equivalents of sodium ascorbate. The reaction is stirred at room temperature for 2 hours.

The peptide is purified, preferably by HPLC, and dried down in a vacuum desiccator.

### EXAMPLE 8

### Synthesis of Aldehyde Neutral Loss Tags

FIG. 19 illustrates a schematic of a synthetic protocol for the production of aldehyde reagents according to an embodiment of this invention. The starting material FMOC-6-amino-hexanol (also referred to as 6-(Fmoc-amino)-1-hexanol; Sigma Aldrich) is converted to the corresponding aldehyde by contacting the alcohol as shown in step (1) of FIG. 19 with IBX-polystyrene (Novabiochem, Merck KGaA, Darmstadt, Germany). Two to four equivalents of resin are used to convert 1 equivalent of alcohol. The reaction is typically conducted in Dichloromethane (DCM) for 4 to 6 hours at room temperature. The spent resin can be filtered away and the resulting aldehyde filtrate can be coupled to a threonine derivatised resin as shown in step (2) of FIG. 19 such as the H-Thr-Gly-NovaSyn resin available from Novabiochem without further purification as the coupling is specific for the aldehyde. Unreacted alcohol can then be washed away.

To couple, the DCM filtrate is diluted with and equal quantity of methanol and then 1% acetic acid is added. This solution is applied to the resin so that there is about 5 equivalents of aldehyde for each equivalent of resin. The reaction is left for 4 hours at room temperature and can be monitored by a TNBS test. The resin is then washed with DCM, DMF (dimethylformamide) and THF (tetrahydrofuran). The secondary amine of resulting oxazolidine must then be BOC-protected, as shown in step (3) of FIG. 19. This can be effected with 5 equivalents of BOC-anhydride with 5 equivalents of NMM in THF. The reaction is left at 50 degrees Celsius for 3 hours. The resin is then washed with DCM, DMF and THF. It is then ready for standard FMOC peptide solid phase synthesis as shown in step (4) of FIG. 19. Peptide Acetyl-Asp-Pro is synthesised on the protected aldehyde linker.

The final deprotection as shown in step (5) of FIG. 19 is as usual for FMOC synthesis but the resin must then be cleaved as shown in step (6) of FIG. 19 by applying three treatments of a mixture of Acetic acid/water/DCM/Methanol (10:5:63:21) and leaving each aliquot for 30 minutes to release the aldehyde peptide tag. This can then be recovered by precipitation, washed, etc. and then purified by HPLC as usual.

The resulting reagents can be used to couple the neutral loss tags to peptides by reductive alkylation as discussed earlier. Reductive alkylation enable the tags to be coupled to epsilon amino groups in polypeptides and peptides with high specificity and yield (Friedman et al., 1974, Int J Pept Protein Res. 6(3):183-185; Chauffe & Friedman, 1977, Adv Exp Med. Biol. 86A:415-424; Geoghegan et al., 1981, Int J Pept Protein Res. 17(3): 345-352; Wong et al., 1984, Anal Biochem. 139(1): 58-67; Cabacungan et al., 1982, Anal Biochem. 124(2): 272-278; Krusemark et al., 2008, Anal Chem. 80(3): 713-720).

In some embodiments, a ketone may be preferred as this will reduce the possibility of multiple labelling of the amino groups in peptides and polypeptides during reductive alkylation.

### EXAMPLE 9

### Synthesis of a Set of Three Peptides Incorporating a Neutral Loss Tag

Three peptides with the sequences shown in Table 5 below (and see also FIGS. 20-22) were synthesised by standard automated FMOC peptide synthesis:

TABLE 5

| Peptide | Sequence |
|---|---|
| “SmallAspPip” | Acetyl-Asp-Pip-Gly-Asn-Thr-Ala-Gly- |
| | Val-Tyr-Thr-Lys [SEQ ID NO: 1] |
| “MediumAspPip” | Acetyl-Asp-Pip-Ile-Ile-Ala-Glu-Gly- |
| | Ala-Asn-Gly-Ala-Thr-Thr-Ala-Glu- |
| | Ala-Glu-Lys [SEQ ID NO: 2] |
| “LargeAspPip” | Acetyl-Asp-Pip-Gly-Leu-Gly-Glu-His- |
| | Asn-Ile-Asp-Val-Leu-Glu-Gly-Asn- |
| | Glu-Phe-Asp-Ile-Asn-Ala-Ala-Lys |
| | [SEQ ID NO: 3] |

where the three-letter code “Pip” refers to the amino acid piperazin-1-ylacetic acid, available as an FMOC reagent for automated peptide synthesis from Sigma-Aldrich.

The peptides were purified by reverse phase HPLC according to standard procedures.

These peptides were designed with a model neutral loss tag pre-incorporated into the peptides at the N-terminus. The tag comprises the N-terminal Acetate-Asp-Pip structure (underlined in Table 5 above). These tagged peptides are designed to demonstrate that it is possible to induce loss of the neutral loss tag at low collision energies without significant fragmentation of the rest of the peptide thus allowing a difference spectrum to be calculated. The expected fragmentation of the three peptides to give the corresponding neutral loss daughter ions is shown in FIGS. 20, 21 and 22. For SmallAspPip [SEQ ID NO: 1], the mass marker labelled peptide [M+H]⁺=1193.6 and [M+2H]²⁺=597.3, while for the peptide from which the mass marker has been fragmented [M+H]⁺=1036.5 and [M+2H]²⁺=518.8. For MediumAspPip [SEQ ID NO: 2], the mass marker labelled peptide [M+H]⁺=1827.9 and [M+2H]²⁺=914.5, while for the peptide from which the mass marker has been fragmented [M+H]⁺=1670.9 and [M+2H]²⁺=835.9. For LargeAspPip [SEQ ID NO: 3], the mass marker labelled peptide [M+H]⁺=2519.2, [M+2H]²⁺=1260.1 and [M+3H]³⁺=840.4 while for the peptide from which the mass marker has been fragmented [M+H]⁺=2362.2, [M+2H]²⁺=1181.6 and [M+3H]³⁺=788.1.

The actual behaviour of the peptides is tested in Electrospray and MALDI mass analysis is discussed in Examples 10 and 11.

### EXAMPLE 10

### Analysis of a Set of Three Peptides Incorporating a Neutral Loss Tag by ESI-MS

The peptides from Example 7 were diluted to 7.5 pmol/μl in water. Aliquots of 10 μl were added to 10 μl of methanol and then adjusted to 1% formic acid. Solutions of the individual peptides and a mixture of all 3 were then analysed by electrospray ionisation mass spectrometry on a Micromass/Waters Q-TOF Micro instrument. FIGS. 23, 24 and 25 show ESI-MS spectra of the peptides SmallAspPip, MediumAspPip and LargeAspPip [SEQ ID NOs 1-3] respectively where the CID voltage in the collision cell has been set to 10V, a low collision energy. In all three cases, the main ion was the [M+2H]²⁺ species of each peptide. It can be seen that all three peptides are relatively stable but SmallAspPip [SEQ ID NO: 1] shows a small amount of the expected fragment ion from the expected neutral loss of the tag. FIGS. 26 and 27 show ESI-MS spectra of the peptides SmallAspPip and MediumAspPip [SEQ ID NOs 1-2] respectively where the CID voltage in the collision cell has been set to 20V, a modest collision energy. It can be seen that SmallAspPip and MediumAspPip [SEQ ID NOs 1-2] show a significant shift from the parent peptide to the daughter peptide resulting from the expected neutral loss of the tag at this collision energy. Note that there is not a significant amount of further fragmentation of the peptide to give b or y ions in SmallAspPip [SEQ ID NO: 1] but a small amount of further fragmentation of the peptide is seen in MediumAspPip [SEQ ID NO: 2] but these are not the main ion peaks, i.e. the neutral loss tag is eliminating without much further fragmentation as required by the methods of this invention. The peptide, LargeAspPip [SEQ ID NO: 3], undergoes fragmentation to give the expected daughter ion at a collision energy of 30 V (FIG. 28) and even then the shift from parent to daughter is far from complete, although the lower intensity ion at m/z 788 corresponding to the 3+ neutral loss species (FIG. 22). Fortunately, the methods of this invention do not depend on the completeness of the fragmentation as detection of peptides with a difference spectrum only relies on the peptides fragmenting to give a measurable difference in the difference spectrum. This means the modest amount of fragmentation in SmallAspPip [SEQ ID NO: 1] will also not be a problem due to the large shift at the higher collision energy.

### EXAMPLE 11

### Analysis of a Set of Three Peptides Incorporating a Neutral Loss Tag by MALDI-Trap-TOF Mass Spectrometry

The peptides from Example 7 were diluted to 750 fmol/μl in water. Aliquots of 1 μl were added to 10 μl of saturated (25 mg/ml) 2,5-dihydroxybenzoic acid (DHB) in 1:1 acetonitrile/water with 0.1% trifluoroacetic acid and then adjusted to 1% formic acid. Solutions of the individual peptides and a mixture of all 3 were spotted down on a metal target and then analysed by MALDI TRAP-TOF mass spectrometry on a Kratos Axima Resonance instrument.

FIGS. 29, 30 and 31 show MALDI-MS/MS spectra of the peptides SmallAspPip, MediumAspPip and LargeAspPip [SEQ ID NOs 1-3] respectively. Each figure shows a series of spectra at increasing CID energies in the TRAP portion of the Axima instrument, which acts as a collision cell. The laser energy has been set to 50 (an arbitrary instrument specific units), representing a relatively low laser power. In the experiments shown in these three figures, each peptide has been specifically isolated in the trap of the Trap-TOF instrument, subjected to increasing CID energies after which the fragments were ejected into the TOF for mass analysis. It can be seen at low collision energies that the peptides adopt the singly protonated, singly charged state almost exclusively, as is typical for MALDI. All three peptides are relatively stable at CID energies below 100 (another arbitrary instrument specific unit) but all three peptides exhibit almost complete elimination of the expected neutral loss tag fragment at an energy of 100. It is important to note that the CID energy in the trap is determined relative to the ion mass-to-charge ratio, i.e. the CID energy of 100 for LargeAspPip [SEQ ID NO: 3] is actually 2.1 times greater (the ratio of their singly charged ion masses). It is also worth noting that the fragmentation of all three ions is almost complete without further fragmentation at this collision energy.

FIG. 32 shows MALDI MS/MS spectra of a mixture of the three peptides SmallAspPip, MediumAspPip and LargeAspPip [SEQ ID NOs 1-3]. Because of the way the trap operates, this series of spectra was generated by a different method from the previous three. The Trap contains a ‘bath gas’ or coolant gas to enable collisional cooling of the ions retained in the Trap. This gas, typically helium, neon or argon, is pulsed into the Trap using a valve and the duration of the pulse can be varied. Varying the collision gas pulse duration regulates the amount of collisional cooling that takes place. Collisional cooling dissipates the energy that is imparted to the ions during CID. More collisional cooling reduces fragmentation, thus a longer pulse time introduces more gas into the Trap and reduces fragmentation for a given excitation energy. This parameter was fixed in the experiments shown in FIGS. 29, 30 and 31 but is varied in FIG. 32 to allow the relative fragmentation of a mixture of peptides to be observed.

FIG. 32 shows that as the duration of the gas pulse is decreased (given as t=52 etc., in units of μs), there is a shift from the parent of all three peptides to the expected daughter peptide ion resulting from the expected neutral loss of the tag. Note that there is not a significant amount of further fragmentation of the peptide to give b or y ions, i.e. the neutral loss tag is eliminating without further fragmentation as required by the methods of this invention. This is a further feature of traps that make them advantageous for the practice of this invention. CID in Traps is achieved by applying an excitation frequency pulse that excites the peptides to fragment, but the presence of the coolant gas dissipates that energy quickly meaning that the lowest energy fragmentation pathways are favoured, particularly with lower energy excitation pulses. Thus it can be seen that the necessary neutral losses of the expected/desired tag fragments can be induced in a controllable fashion on a suitable instrument, even though the collisional energies required for complete fragmentation vary in proportion to the size of the ions. Difference spectra could be calculated by normalizing the ion intensities of the individual spectra in FIG. 32 against their total ion currents. These normalized spectra could then be subtracted from each other to give the difference spectrum between different collision energies.

It is noted that there is some spontaneous neutral loss of expected tag fragments due to the laser excitation process in MALDI but this tends to give a steady ratio of tag loss that would not affect the final result due to the much larger loss of tag induced by higher Collision energies.

Although the present invention has been described with reference to preferred or exemplary embodiments, those skilled in the art will recognise that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

All documents cited herein are incorporated by reference in their entirety.

What is claimed is:

1. A mass marker for labelling of an analyte detectable by mass spectrometry such as neutral loss mass spectroscopy, in which the mass marker comprises a neutral loss mass modifier linked via a first collision cleavable linker to a reactive group having reactive functionality for attachment to the analyte, and in which the neutral loss mass modifier upon cleavage from the analyte during mass spectroscopy is neutral.

2. The mass marker according to claim 1, in which the neutral loss mass modifier upon cleavage from the analyte allows a charge to remain on the analyte such that the analyte is detectable by mass spectrometry.

3. The mass marker according to claim 1, in which the neutral loss mass modifier does not localise a charge onto itself during mass spectrometry, for example during and/or after ionisation.

4. The mass marker according to claim 1, further comprising a neutral loss mass normaliser positioned between the first collision cleavable linker and the reactive group.

5. The mass marker according to claim 4, in which the neutral loss mass normaliser does not localise a charge onto itself during mass spectrometry, for example during ionisation.

6. The mass marker according to claim 4, further comprising a second collision cleavable linker positioned between the neutral loss mass normaliser and the reactive group.

7. The mass marker according to claim 6, in which the first collision cleavable linker is cleavable at a lower collision energy that the second collision cleavable linker.

8. The mass marker according to claim 6, in which each collision cleavable linker is cleavable by collision induced dissociation (CID) during mass spectrometry at a low collision energy.

9. The mass marker according to claim 1, in which the analyte is a biomolecule.

10. The mass marker according to claim 9, in which the biomolecule is a peptide or a polypeptide.

11. The mass marker according to claim 9, in which the biomolecule is a nucleic acid.

12. The mass marker according to claim 9, in which the neutral loss mass modifier or a precursor thereof comprises any of the group consisting of: one or more amino acids and/or isotopically modified forms thereof (for example, one or more of the isotopically modified amino acids provided in Table 3), amino-butyric acid and/or one or more isotopes thereof, a fluorinated amino acid (for example, one or more of the fluorinated amino acid shown in Table 4) and/or one or more isotopes thereof, a dipeptide consisting of proline and aspartic acid (for example, proline-aspartic acid or aspartic acid-proline) and/or one or more isotopes thereof, a dipeptide consisting of proline and an aliphatic amino acid (for example, an alanine-proline dipeptide) and/or one or more isotopes thereof, and a molecule comprising piperazine optionally with one or more constituent substitutions (for example, piperazine-1-ylacetate) and/or one or more isotopes thereof.

13. The mass marker according to claim 1, in which the collision cleavable linker comprises an amide group formed by reacting a primary amine group of an amino acid or an amino-derivatised oligonucleotide of a mass marker precursor with a dicarboxylic anhydride (such as succinic anhydride, maleic anhydride, citraconic anhydride, dimethyl maleic anhydride, phthalic anhydride, and/or exo-cis-3,6-endoxo-delta-tetrahydrophthalic anhydride) or an isotope thereof.

14. The mass marker according to any claim 1, in which the reactive group or a precursor thereof comprises or is a thiol.

15. The mass marker according to claim 14, in which the reactive group or a precursor thereof comprises or is a cysteine residue and/or a modified cysteine residue.

16. The mass marker according to claim 14, in which the thiol is used to label a dehydroalanine group and/or a methyldehydroalanine group of an analyte.

17. The mass marker according to claim 1, in which the reactive group or a precursor thereof comprises an amino group.

18. The mass marker according to claim 17, in which the reactive group or a precursor thereof comprises or is a lysine residue and/or a modified lysine residue.

19. The mass marker according to claim 17, in which the amino group is reacted in the presence of a carbodiimide to allow coupling of the mass marker to free carboxyl functionalities on an analyte.

20. The mass marker according to claim 16, in which the amino group is reacted with a carbonyl group of an analyte by reductive alkylation.

21. (canceled)

22. The mass marker according to claim 14, in which the thiol reactive group comprises an iodacetyl moiety.

23. The mass marker according to claim 14, in which the thiol reactive group comprises a Michael reagent.

24. The mass marker according to claim 1, in which the reactive group or a precursor thereof comprises or is an amine reactive group.

25. The mass marker according to claim 24, in which the amine reactive group or a precursor thereof comprises or is an aldehyde group.

26. The mass marker according to claim 24, in which the amine reactive group or a precursor thereof comprises or is a ketone group.

27. The mass marker according to claim 24, in which the mass maker is used to label an amino group of an analyte by reductive alkylation.

28. The mass marker according to claim 24, in which the amine reactive group is a guanidination reagent, for example O-methylisourea.

29. (canceled)

30. The mass marker according to claim 24, in which the amine reactive group comprises or is an N-hydroxysuccinamide ester.

31. The mass marker according to claim 30, in which the reactive group or a precursor thereof comprises or is a hydrazide.

32. The mass marker according to claim 1, in which the mass marker or components thereof or a precursor of either are produced using a peptide synthesiser, for example an automated peptide synthesiser.

33. The mass marker according to claim 1, further comprising a spacer molecule between the neutral loss mass modifier and the reactive group.

34. The mass marker according to claim 33, in which the spacer molecule is formed by insertion of aminohexanoic acid into a mass marker precursor.

35. The mass marker according to claim 34, further comprising an affinity capture ligand, for example any one or more of the group consisting of: biotin, digoxigenin, fluorescein, a nitrophenyl moiety, a peptide epitope (for example, a c-myc epitope), oligomeric histidine (for example, hexahistidine), and a boronic acid-containing ligand (for example, phenylboronic acid).

36. A set of two or more mass markers as defined in claim 1.

37. The set according to claim 36, in which each mass marker in the set is chemically identical.

38. The set according to claim 36, in which each mass marker in the set has a different molecular mass from other mass markers in the set.

39. The set according to claim 38, in which the mass markers have different molecular masses due to the presence of one or more different isotopes in the neutral loss mass modifier of each mass marker and/or, where present, the neutral loss mass normaliser of each mass marker.

40. The set according to claim 39, in which the isotopes are stable isotopes such as for example any one or more of the group consisting of ²H, ¹³C, ¹⁵N, ¹⁷0, ¹⁸O and ³⁴S.

41. The set according to claim 36, in which each mass marker in the set has the same molecular mass as other mass markers in the set but each mass marker has a unique internal molecular mass distribution.

42. The set according to claim 41, in which the unique internal molecular mass distribution of each marker is formed by differences in the molecular masses of the neutral loss mass modifier and the neutral loss mass normaliser between mass markers.

43. The set according to claim 42, in which the neutral loss mass modifiers and the neutral loss mass normalisers of the mass markers are isotopically modified.

44. An array of mass markers comprising one or more mass markers as defined in claim 1, or comprising one or more sets of mass markers as defined in claim 36, in which the mass markers are resolvable in a compressed mass range.

45. The array according to claim 44, in which the mass markers do not interfere substantially with separation processes such as electrophoresis or chromatographic separations.

46. A method for identifying an analyte of interest in a sample using mass spectroscopy, comprising the steps of:
1) labelling the analyte in the sample with a mass marker as defined in claim 1 to form a labelled analyte sample;
2) subjecting the labelled analyte sample to mass spectrometry (for example, in MS-mode) at a first collision energy and obtaining a first spectrum;
3) subjecting the labelled analyte sample to mass spectrometry (for example, in MS-mode) at a second collision energy at which the mass marker is cleaved from the labelled analyte, in which the second collision energy is optionally higher than the first collision energy, and obtaining a second spectrum;
4) calculating a difference spectrum between first and second spectra;
5) comparing a region of the difference spectrum and a template corresponding to predicted mass differences generated by labelled and unlabelled analyte; and
6) identifying and scoring characteristic shifts in mass-to-charge ratios of the analyte of interest based on the comparison in step 5).

47. The method according to claim 46, in which step 4) includes normalisation of results obtained from the first and second spectra prior to calculation of the difference spectrum.

48. The method according to claim 46, in which step 5) includes adjusting and/or scaling the template to correspond with intensity and/or peaks in the difference spectrum.

49. The method according to claim 46, in which steps 5) and 6) are repeated by comparing a different region of the difference spectrum with the template.

50. A method of determining the relative abundance of one or more analytes in two or more samples using mass spectroscopy, comprising the steps of:
1) reacting a first sample with a first isotope of a mass marker as defined in claim 1 to form a first labelled sample;
2) reacting a second (or further) sample(s) with a second (or further) different isotope(s) of a mass marker as defined in claim 1 to form a second (or further) labelled sample(s);
3) pooling the first and second (or further) labelled samples to form a pooled labelled sample; and 4) subjecting the pooled sample to mass spectrometry and obtaining a mass spectrum to determine relative abundancies of analyte(s) present in the first and second (or further) samples.

51. The method according to claim 50, in which the mass markers are non-isobaric.

52. The method according to claim 50, in which the mass markers are isobaric.

53. The method according to claim 52, in which the method comprises a further step following mass spectrometry of selecting a population of the pooled labelled sample for further analysis and/or fragmentation and/or isolation.

54. A method of analysing an analyte such as a biomolecule or a mixture of analytes such as biomolecules using mass spectroscopy, comprising the steps of:
1) reacting the analyte or mixture of analytes with a mass marker as defined in claim 1 to form one or more labelled analytes;
2) optionally, separating the one or more labelled analytes (for example, in one or more separation steps, such as using electrophoretisis and/or chromatography);
3) ionising the one or more labelled analytes;
4) selecting ions of a predetermined mass to charge ratio corresponding to the mass to charge ratio of the preferred ions of the one or more labelled analytes in a mass analyser;
5) inducing dissociation of the selected ions by collision to form collision products; and
6) detecting the collision products to identify one or more analyte ions that are generated by neutral loss of the mass modifier.

55. The method according to claim 54, in which the mass markers comprise an affinity tag and in which the method comprises a further step of capturing an affinity-tagged labelled analyte or analytes by a counter-ligand to allow labelled analyte(s) to be separated from unlabelled analyte(s).

56. A method for analysing a sample containing one or more polypeptides having one or more cysteine residues using mass spectrometry, comprising the steps of:
1) cleaving the polypeptides with a sequence-specific endoprotease,
2) reducing and reacting cysteine residues with a mass marker as defined in of claim 1 and having a thiol-reactive affinity ligand to form labelled peptides,
3) capturing labelled peptides onto an avidin derivatised solid support, and
4) analysing the captured labelled peptides by mass spectrometry.

57. The method of claim 56, in which steps 1) and 2) are performed in either order or simultaneously.

58. The method of claim 56, in which the sequence-specific endoprotease is Lys-C or trypsin.

59. A method for analysing a sample comprising one or more polypeptides by mass spectroscopy, comprising the steps of:
1) cleaving the polypeptides with a sequence-specific endoprotease (such as Lys-C) that cleaves immediately C-terminal to any Lysine residues present in the polypeptides, thereby forming peptide fragments each having a C-terminus with a free epsilon amino group and an N-terminus with a free alpha amino group;
2) labelling the free epsilon amino group and/or the free alpha amino group with a mass marker, for example as defined in claim 1, to form labelled peptides; and
3) analysing the labelled peptides by mass spectrometry.

60. The method according to claim 59, comprising a further step 2A) after step 2) and before step 3) of cleaving the labelled peptides with a sequence-specific endoprotease (such as trypsin or Arg-C) that cleaves immediately C-terminal to any Arginine residues present in the peptides.

61. The method according to claim 59, in which the mass marker is a neutral loss mass marker as defined in claim 1.

62. The method according to claim 59, in which step 3) comprises shotgun peptide sequencing.

63. The method according to claim 59, comprising a further step 4) of isolating labelled peptides comprising an affinity capture ligand using affinity capture.

64. A method for analysing a sample comprising carbohydrate-modified proteins, comprises the steps of:
1) treating the sample with a sequence-specific cleavage reagent (such as trypsin or Lys-C) to form peptides having a free alpha amino group;
2) passing the peptides through an affinity column (such as an affinity column containing lectins or boronic acid derivatives) to capture carbohydrate-modified peptides;
3) labelling the captured carbohydrate-modified peptides at their free alpha amino group with a mass marker as defined in claim 1 to form labelled peptides; and
4) analysing the labelled peptides by mass spectrometry.

65. The method according to claim 64, in which the sequence-specific cleavage reagent used in step 2) is Lys-C, producing peptides having a free epsilon-amino group and a free alpha amino group.

66. The method according to claim 65, in with both the free epsilon-amino group and the free alpha amino group of the peptides are labelled in step 3).

67. A method of analysing a sample containing carbohydrate-modified polypeptides, comprises the steps of:
1) treating the sample with periodate to allow carbohydrates with vicinal cis-diols on glycopeptides to gain a carbonyl functionality;
2) labelling the carbonyl functionality with a mass marker as defined in claim 1 which has been hydrazide-activated, to form labelled polypeptides;
3) treating the labelled polypeptides with a sequence-specific endoprotease to form labelled peptides; and
4) analysing the labelled peptides by mass spectrometry.

68. The method according claim 67, in which steps 2) and 3) are performed in either order or simultaneously.

69. (canceled)

70. (canceled)
